(19) United States
(12) Patent Application Publication
(10) Pub. No.: US 2009/0239229 A1
(43) Pub. Date: Sep. 24, 2009

(54) DNA Repair Proteins Associated With Triple Negative Breast Cancers and Methods of Use Thereof

(75) Inventors: David Weaver, Newton, MA (US); Kam Marie Sprott, Needham, MA (US); XioaZhe Wang, Auburndale, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: DNAR, INC, Cambridge, MA (US)

(21) Appl. No.: 12/405,028

(22) Filed: Mar. 16, 2009

Related U.S. Application Data
(60) Provisional application No. 61/069,487, filed on Mar. 14, 2008.
(60) Provisional application No. 61/128,776, filed on May 23, 2008.

(51) Int. Cl. C12Q 1/68; C12Q 1/02; C12Q 1/48; G01N 33/573

(52) U.S. Cl. 435/6; 435/29; 435/15; 435/7.92; 977/920; 977/774

(57) ABSTRACT

The present invention provides methods of detecting triple negative breast cancer recurrence using biomarkers.

### RELATED APPLICATIONS

This application claims the benefit of U.S. Ser. No. 61/069,487 filed Mar. 14, 2008 and U.S. Ser. No. 61/128,776 filed May 23, 2008 the contents of which are incorporated by reference in their entireties.

### FIELD OF THE INVENTION

The present invention relates generally to the identification of biomarkerss and methods of using such biomarkers in the screening, prevention, diagnosis, therapy, monitoring, and prognosis of triple negative breast cancer.

### BACKGROUND OF THE INVENTION

Triple negative breast cancer, those that are estrogen receptor (ER) negative, progesterone receptor (PR) negative, and Her-2 negative comprise approximately 15% of all breast cancers and have an aggressive clinical course with high rates of local and systemic relapse. The clinical course reflects the biology of the tumor as well as the absence of conventional targets for treatment such as hormonal therapy for ER or PR positive patients and trastuzumab for Her-2 over-expressing tumors. In addition, these cancers may have different sensitivity to chemotherapeutic agents². As such, there is a great deal of interest in determining novel therapeutic regimens for this aggressive disease. Whereas triple negative breast cancers are an established subtype of breast cancer, relatively little biomarker information is available for patient stratification and to direct treatment decisions.

DNA repair deficits may be a characteristic of triple negative cancers. These tumors exhibit more DNA copy alterations and loss of heterozygosity⁴ than other breast cancers, features suggestive of genomic instability. Furthermore, sporadic triple negative tumors share phenotypic and cytogenetic features with familial BRCA1 associated cancer and segregate strongly with BRCA1 cancers using microarray RNA expression data. BRCA1 mutant tumors are thought to be deficient in DNA repair, particularly homologous recombination, and these similarities may suggest that a similar DNA repair deficiency may underlie the development of triple negative tumors. Possible deficits in DNA repair do not only have implications for response to current therapy but also with respect to novel targeted therapies.

### SUMMARY OF THE INVENTION

The present invention relates in part to the discovery that certain biological markers (referred to herein as “TNBCMARKERS”), such as proteins, nucleic acids, polymorphisms, metabolites, and other analytes, as well as certain physiological conditions and states, are present or altered in subjects with an increased risk of developing a recurrent triple negative breast cancer.

Accordingly in one aspect the invention provides a method with a predetermined level of predictability for assessing a risk of development of a triple negative breast cancer or a recurrence of triple negative breast cancer in a subject. Risk of developing triple negative breast cancer or a recurrence of triple negative breast cancer is determined by measuring the level of an effective amount of a TNBCMARKER in a sample from the subject. An increased risk of developing triple negative breast cancer or a recurrence of triple negative breast cancer in the subject is determined by measuring a clinically significant alteration in the level of the TNBCMARKER in the sample. Alternatively, an increased risk of developing triple negative breast cancer or a recurrence of triple negative breast cancer in the subject is determined by comparing the level of the effective amount TNBCMARKER to a reference value. In some aspects the reference value is an index.

In another aspect the invention provides a method with a predetermined level of predictability for assessing the progression of a triple negative breast cancer in a subject by detecting the level of an effective amount a TNBCMARKERS in a first sample from the subject at a first period of time, detecting the level of an effective amount of TNBCMARKERS in a second sample from the subject at a second period of time and comparing the level of the TNBCMARKERS detected in to a reference value. In some aspects the first sample is taken from the subject prior to being treated for the triple negative breast cancer and the second sample is taken from the subject after being treated for the cancer.

In a further aspect the invention provides a method with a predetermined level of predictability for monitoring the effectiveness of treatment or selecting a treatment regimen for triple negative breast cancer by detecting the level of an effective amount of TNBCMARKERS in a first sample from the subject at a first period of time and optionally detecting the level of an effective amount of TNBCMARKERS in a second sample from the subject at a second period of time. The level of the effective amount of TNBCMARKERS detected at the first period of time is compared to the level detected at the second period of time or alternatively a reference value. Effectiveness of treatment is monitored by a change in the level of the effective amount of TNBCMARKERS from the subject.

A TNBCMARKER includes for example FANCD2, XPF, pMK2, PAR, PARP1, MLH1, ATM, RAD51, BRCA1, ERCC1, NQO1, p53, Ki67. One, two, three, four, five, ten or more TNBCMARKERS are measured. Preferably, at least two TNBCMARKERS selected from FANCD2, XPF, pMK2, PAR, PARP1, MLH1, ATM, RAD51, BRCA1, and ERCC1, are measured. In some aspects FANDC2, BRCA1, or RAD51 and at least one TNBCMARKER selected from XPF or ERCC1; pMK2 or ATM; or PAR or PARP1 is measured.

In a further aspect the TNBCMARKERS are DNA repair proteins belonging to different DNA repair pathways. Alternatively three or more TNBCMARKERS are measures where TNBCMARKERS belonging to two or more different DNA repair pathways.

In other aspects of the invention FAND2 and at least one TNBCMARKER selected from XPF, pMK2, PAR, PARP1, MLH1, ATM, RAD51, BRCA1, and ERCC1 is measured; XPF and at least one TNBCMARKER selected from FANCD2, pMK2, PAR, PARP1, MLH1, ATM, RAD51, BRCA1, and ERCC1 is measured; pMK2 and at least one TNBCMARKER selected from FANCD2, XPF, PAR, PARP1, MLH1, ATM, RAD51, BRCA1, and ERCC1 is measured; PAR and at least one TNBCMARKER selected from FANCD2, XPF, pMK2, PARP1, MLH1, ATM, RAD51, BRCA1, and ERCC1 is measured; PARP1 and at least one TNBCMARKER selected from FANCD2, XPF, pMK2, PAR, MLH1, ATM, RAD51, BRCA1, and ERCC1; MLH1 and at least one TNBCMARKER selected from FANCD2, XPF, pMK2, PAR, PARP1, ATM, RAD51, BRCA1, and ERCC1 is measured; ATM and at least one TNBCMARKER selected from FANCD2, XPF, pMK2, PAR, PARP1, MLH1, RAD51, BRCA1, and ERCC1 is measured; RAD51 and at least one TNBCMARKER selected from FANCD2, XPF, pMK2, PAR, PARP1, MLH1, ATM, BRCA1, and ERCC1 is measured; BRCA1 and at least one FANCD2, XPF, pMK2, PAR, PARP1, MLH1, ATM, RAD51, and ERCC1 is measured; or ERCC1 and at least one FANCD2, XPF, pMK2, PAR, PARP1, MLH1, ATM, RAD51, and BRCA1 is measured. Optionally one or more TNBCMARKERS selected from NQO1, p53, and Ki67 is additionally measured.

Optionally, the methods of the invention further include measuring at least one standard parameters associated with a tumor.

The level of a TNBCMARKER is measured electrophoretically or immunochemically. For example the level of the TNBCMARKER is detected by radioimmunoassay, immunofluorescence assay or by an enzyme-linked immunosorbent assay.

The subject has a triple negative breast cancer, or a recurrent triple negative breast cancer. In some aspects the sample is taken for a subject that has previously been treated for triple negative breast cancer. Alternatively, the sample is taken from the subject prior to being treated for triple negative breast cancer. The sample is a tumor biopsy such as fine needle aspirate a core biopsy, an excisional tissue biopsy or an incisional tissue biopsy. The sample is a tumor cell form blood, lymph nodes or a bodily fluid.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. Immunohistochemistry patterns for triple negative breast cancer specimens. A, FANCD2. The staining pattern of FANCD2 is recognizable as nuclear foci, indicative of activation of the FANCD2 pathways that stimulates homologous recombination. B, pMK2. Four representative cancer cores are displayed demonstrating the four recognized patterns of phosphoMapkapkinase2 (pMK2) in triple negative breast cancer tumor zones.

FIG. 2. Marker output variations between patients far exceed the inter-sample variability in triple negative breast cancer. A, Theoretical definition of the calculation for core-core variability and rank change assessment; B, Table indicating the average error and N number of patients being evaluated for TNBCMARKERS; C, Results from patient ranking for four TNBCMARKERS. Patient marker scores are sorted from lowest to highest, and core-core variance per patient is displayed as a vertical dashed line.

FIG. 3. Separation of patients into recurrence groups from single TNBCMARKERS partition analysis. Patients are separated by partition analysis in evaluation of their Time to Recurrence. Examples shown are DNA repair markers from the list in Table 1, XPF, FANCD2, PAR, and PMK2. Dotted line demarcates a separation between the recurrence groups.

FIG. 4. Two marker models demonstrate that both markers are important in discriminating the two recurrence groups. Shown are six examples from four markers in pairwise combinations by binary analysis. Triangles, Early Recurrence group; Circles, Late Recurrence group. Patients are separated by partition analysis. Dotted line indicates a demarcation of separation between the recurrence groups

FIG. 5. Second group demonstration for two marker models Group 2 consists of additional markers in the study, PARP1, MLH1, Ki67. Patients are separated by partition analysis. Dotted line indicates a demarcation of separation between the recurrence groups.

FIG. 6. Threshold marker values for four TNBCMARKERS Four TNBCMARKERS (XPF, FANCD2, PAR, PMK2) are shown for marker levels and patient indices. Patients are ranked from lowest marker score to highest (left to right). Line indicates maximizing cutoff between the two Recurrence groups (no recurrence, equivalent to Late Recurrence) and (recurrence, equivalent to Early Recurrence). The threshold values as absolute marker values are listed in the table insert.

FIG. 7. A four DNA repair marker algorithm significantly separates triple negative breast cancer patients into early recurrence and late recurrence groups. A, Training dataset; Lines denote the Time to recurrence profile and recurrence-free proportion for an Early Recurrence patient subset and a Late Recurrence patient subset as labeled and defined by the test. ALL PATIENTS and Recurrence-Free proportion over Time is shown by the dashed line. B, Test dataset. The test dataset are patients not previously analyzed by the marker training and algorithm exercises. ALL PATIENTS and Recurrence-Free proportion over Time is shown by the dashed line.

FIG. 8. Comparison of Training and Test datasets regarding the identification of Recurrence groups. The Early Recurrence and Late Recurrence groups were compared for the Training and Test datasets (solid lines) with the 95% confidence intervals of the separation noted (dotted lines). For these comparisons, the Non-recurrent (Late) group is not statistically different between Training and Test sets (p=0.606). Likewise, the Recurrent (Early) group is not statistically different between Training and Test sets (p=0.625).

FIG. 9. Relative Risk and Apparent Error Rate is superior for a four DNA repair marker model. A, Training dataset, B, Test dataset. Relative risk is a ratio of the probability of the recurrence occurring between the High Score Recurrence group (Good Survival) and Low Score Recurrence group (Poor Survival). Apparent error rate (AER) is the fraction of patients misclassified by the combined score.

FIG. 10. Root marker performance improved in multimarker models. Three Root markers, FANCD2, XPF, and RAD51, are shown. In each case, the computed log 10 P-value (squares), Positive Predictive Value (PPV) (triangles) and AER (black circles) are shown for each Root Marker alone, and in combination with other TNBCMARKERS in 2-, 3- and 4-marker models. The median value of all the models are plotted for each model.

FIG. 11. Probability Analysis Schematic. Probability analysis is an algorithm that allows for a continuous scoring of the TNBCMARKER outputs. In the algorithm, a region of low incidence of recurrence and a region of high incidence of recurrence is proposed from estimates of the probability density distributions. For the Early Recurrence (ie. likely to recur) and Late Recurrence (ie. not likely to recur) groups, a single score reflecting group membership is constructed from the individual group probabilities.

FIG. 12. Partition Analysis of the DNA Repair TNBCMARKERS on all 1-, 2-, 3-, and 4-TNBCMARKER models. The markers in the analysis included the group of DNA Repair markers (XPF, pMK2, PAR, PARP1, MLH, FANCD2, ATM, RAD51, BRCA1, ERCC1, and NQO1). All 1-marker, 2-marker, 3-marker, and 4-marker combination models were compared and plotted on x-axis as 1, 2, 3, 4. The median value of all models in the group is represented by a narrow white box is the center region of each plotted value. Black box denotes 95% confidence interval for the median. Outside white box denotes the middle half of the data (white part above median is quarter of data, white part below median is quarter of data. For partition analysis, the outputs for P-value, Relative Risk, Positive Predictive Value, Specificity, AER were compared.

FIG. 13. Probability Analysis of a Single Marker, XPF. Scores by Outcome, patients are separated by those with an event (Recurrence) or no event (No Recurrence) and the probability of correctly calling the result of the test with the marker is plotted from a scale of −1.0 to +1.0., Kaplan-Meier Recurrence Curves, LATE and EARLY refer to the patient subgrouping into Late Time to Recurrence (Good Outcome) and Early Time to Recurrence (Poor Outcome) respectively. Predicted Outcome from Score, is shown by plotting the likelihood of an event (Recurrence) against the probability score (95% confidence intervals with dashed lines); ROC Plot from Score, Area Under Curve (AUC) sensitivity/specificity determination listed, values range from 0-1.

FIG. 14. Probability Analysis of a Single Marker, FANCD2. Scores by Outcome, patients are separated by those with an event (Recurrence) or no event (No Recurrence) and the probability of correctly calling the result of the test with the marker is plotted from a scale of −1.0 to +1.0., Kaplan-Meier Recurrence Curves, LATE and EARLY refer to the patient subgrouping into Late Time to Recurrence (Good Outcome) and Early Time to Recurrence (Poor Outcome) respectively. Predicted Outcome from Score, is shown by plotting the likelihood of an event (Recurrence) against the probability score (95% confidence intervals with dashed lines); ROC Plot from Score, Area Under Curve (AUC) sensitivity/specificity determination listed, values range from 0-1.

FIG. 15. Probability Analysis of a Single Marker, PAR. Scores by Outcome, patients are separated by those with an event (Recurrence) or no event (No Recurrence) and the probability of correctly calling the result of the test with the marker is plotted from a scale of −1.0 to +1.0., Kaplan-Meier Recurrence Curves, LATE and EARLY refer to the patient subgrouping into Late Time to Recurrence (Good Outcome) and Early Time to Recurrence (Poor Outcome) respectively. Predicted Outcome from Score, is shown by plotting the likelihood of an event (Recurrence) against the probability score (95% confidence intervals with dashed lines); ROC Plot from Score, Area Under Curve (AUC) sensitivity/specificity determination listed, values range from 0-1.

FIG. 16. Probability Analysis of a Three Marker Model—XPF, FANCD2, PAR. Scores by Outcome, patients are separated by those with an event (Recurrence) or no event (No Recurrence) and the probability of correctly calling the result of the test with the three marker test is plotted from a scale of −1.0 to +1.0., Kaplan-Meier Recurrence Curves, LATE and EARLY refer to the patient subgrouping into Late Time to Recurrence (Good Outcome) and Early Time to Recurrence (Poor Outcome) respectively. Predicted Outcome from Score, is shown by plotting the likelihood of an event (Recurrence) against the probability score (95% confidence intervals with dashed lines); ROC Plot from Score, Area Under Curve (AUC) sensitivity/specificity determination listed, values range from 0-1.

FIG. 17. Probability Analysis of a Four Marker Model—XPF, FANCD2, PAR, PMK2. Scores by Outcome, patients are separated by those with an event (Recurrence) or no event (No Recurrence) and the probability of correctly calling the result of the test with the four marker test is plotted from a scale of −1.0 to +1.0., Kaplan-Meier Recurrence Curves, LATE and EARLY refer to the patient subgrouping into Late Time to Recurrence (Good Outcome) and Early Time to Recurrence (Poor Outcome) respectively. Predicted Outcome from Score, is shown by plotting the likelihood of an event (Recurrence) against the probability score (95% confidence intervals with dashed lines); ROC Plot from Score, Area Under Curve (AUC) sensitivity/specificity determination listed, values range from 0-1.

FIG. 18. Probability Analysis of the DNA Repair TNBCMARKERS on all 1-, 2-, 3-, 4-, and 5-TNBCMARKER models. The markers in the analysis included the group of DNA Repair markers (XPF, pMK2, PAR, PARP1, MLH, FANCD2, ATM, RAD51, BRCA1, ERCC1, and NQO1). All 1-marker, 2-marker, 3-marker, 4-, and 5-marker combinations were compared and plotted on x-axis as 1, 2, 3, 4.5. The median value of all models in the group is represented by a narrow white box is the center region of each plotted value. Black box denotes 95% confidence interval for the median. Outside white box denotes the middle half of the data (white part above median is quarter of data, white part below median is quarter of data. The statistical values assessed were Fraction Sample Assigned, AUC, Sensitivity, and Specificity,

FIG. 19. Partition analysis combinations of DNA Repair TNBCMARKERS with NQO1 marker in 2- and 3-marker algorithms. The NQO1 marker values were computed for p-value, Relative Risk, AER, and Sensitivity either singly or in every 2-, and 3-marker model.

### DETAILED DESCRIPTION OF THE INVENTION

The present invention relates to the identification of biomarkers associated with triple negative breast cancer. Specifically, these biomarkers are proteins associated in DNA repair pathways. DNA repair pathways are important to the cellular response network to chemotherapy and radiation.

There are six major DNA repair pathways distinguishable by several criteria which can be divided into three groups those that repair single strand damage and those that repair double stand damage. Single stranded damage repair pathways include Base-Excision Repair (BER); Nucleotide Excision Repair (NER); Mismatch Repair (MMR); Homologous Recombination/Fanconi Anemia pathway (HR/FA); Non-Homologous Endjoining (NHEJ), and Translesion DNA Synthesis repair (TLS).

BER, NER and MMR repair single strand DNA damage. When only one of the two strands of a double helix has a defect, the other strand can be used as a template to guide the correction of the damaged strand. In order to repair damage to one of the two paired molecules of DNA, there exist a number of excision repair mechanisms that remove the damaged nucleotide and replace it with an undamaged nucleotide complementary to that found in the undamaged DNA strand. BER repairs damage due to a single nucleotide caused by oxidation, alkylation, hydrolysis, or deamination. NER repairs damage affecting longer strands of 2-30 bases. This process recognizes bulky, helix-distorting changes such as thymine dimers as well as single-strand breaks (repaired with enzymes such UvrABC endonuclease). A specialized form of NER known as Transcription-Coupled Repair (TCR) deploys high-priority NER repair enzymes to genes that are being actively transcribed. MMR corrects errors of DNA replication and recombination that result in mispaired nucleotides following DNA replication.

NEHJ and HR repair double stranded DNA damage. Double stranded damage is particularly hazardous to dividing cells. The NHEJ pathway operates when the cell has not yet replicated the region of DNA on which the lesion has occurred. The process directly joins the two ends of the broken DNA strands without a template, losing sequence information in the process. Thus, this repair mechanism is necessarily mutagenic. However, if the cell is not dividing and has not replicated its DNA, the NHEJ pathway is the cell's only option. NHEJ relies on chance pairings, or microhomologies, between the single-stranded tails of the two DNA fragments to be joined. There are multiple independent “failsafe” pathways for NHEJ in higher eukaryotes. Recombinational repair requires the presence of an identical or nearly identical sequence to be used as a template for repair of the break. The enzymatic machinery responsible for this repair process is nearly identical to the machinery responsible for chromosomal crossover during meiosis. This pathway allows a damaged chromosome to be repaired using the newly created sister chromatid as a template, i.e. an identical copy that is also linked to the damaged region via the centromere. Double-stranded breaks repaired by this mechanism are usually caused by the replication machinery attempting to synthesize across a single-strand break or unrepaired lesion, both of which result in collapse of the replication fork.

Translesion synthesis is an error-prone (almost error-guaranteeing) last-resort method of repairing a DNA lesion that has not been repaired by any other mechanism. The DNA replication machinery cannot continue replicating past a site of DNA damage, so the advancing replication fork will stall on encountering a damaged base. The translesion synthesis pathway is mediated by specific DNA polymerases that insert extra bases at the site of damage and thus allow replication to bypass the damaged base to continue with chromosome duplication. The bases inserted by the translesion synthesis machinery are template-independent, but not arbitrary; for example, one human polymerase inserts adenine bases when synthesizing past a thymine dimer.

Both normal cellular processes and exogenous agents contribute to the accumulation of DNA damage for which eukaryotic cells have evolved complex and redundant repair mechanisms to ensure stability and high fidelity replication of the genetic material. While spontaneous mutations cannot entirely account for the lifetime cancer risk, defects in DNA repair can lead to a ‘mutator’ phenotype where cells accumulate damage at an accelerated rate, leading to oncogenesis. While these defects may contribute to genomic instability and aggressiveness, they might also sensitize tumor cells to damage by exogenous DNA damaging agents such as chemotherapy and ionizing radiation. Thus, because DNA damage repair defects are more likely to be prevalent in cancer cells and relate to aggressiveness, the cellular DNA repair machinery offers an opportunity for prediction and prognosis as well as a set of targets for therapeutic development.

Triple negative breast cancers are even more likely to harbor deficits in DNA repair. One study used loss of heterozygosity (LOH) as a marker for genomic instability and found that basal-like breast cancers had the highest rate of LOH of all breast cancer subtypes. Furthermore 5q11, near a number of DNA repair and checkpoint genes, was lost in 100% of basal like cancers and never in other subtypes. There is also a high degree of DNA copy gains and losses associated with the basal-like subtype when analyzed by genome-wide array-based comparative genomic hybridization. Familial BRCA1 related cancers also share many clinical and phenotypic features with triple negative cancers, including high grade, EGFR expression, p53 mutations, and cytogenetic abnormalities in addition to ER, PR and Her2 negativity. The BRCA1 protein is involved in DNA repair through its association with homologous recombination in response to DNA double strand breaks.

In this study described herein, representatives from several of these pathways were investigated for associations with clinical outcome of individuals with triple negative breast cancer. Selected DNA repair protein epitopes, NQO1, p53, and Ki67 proteins were evaluated in serial sections from a triple negative breast cancer tissue microarray (TMA). The DNA repair protein epitopes evaluated included XPF and ERCC1 (nucleotide excision repair), FANCD2 (Fanconi Anemia pathway), RAD51 and BRCA1 (homologous recombination), MLH1 (mismatch repair), PARP1 (base excision repair), PAR (base excision repair), and pMK2 (phosphoMapkapKinase2), ATM (DNA damage response). The marker NQO1 is a detoxification enzyme that is shown to associated with sensitivity to anthracycline-based treatments in breast cancer. The marker, Ki67, which localizes in the nucleus, is not a DNA repair marker, but instead is an indicator of cell proliferation capacity within the tumor zone. The marker p53, is a tumor suppressor that is frequently mutated in cancer, and p53 mutations is evidenced by DNA tests or stabilized p53 mutant proteins in immunohistochemistry.

As described in the EXAMPLE section below, the DNA repair biomarkers studied were associated with shorter time to cancer recurrence. Specifically, two, three and four marker model was able to segregate high risk and low risk groups based upon time to recurrence in both the training and test cohorts.

Accordingly, the invention provides methods for identifying subjects who have triple negative breast cancer, or who at risk for experiencing a recurrence of a triple negative breast cancer by the detection of protein biomarkers associated with the triple negative breast cancer. These TNBCMARKERs are also useful for monitoring subjects undergoing treatments and therapies for triple negative breast cancer, and for selecting or modifying therapies and treatments that would be efficacious in subjects having triple negative breast cancer, wherein selection and use of such treatments and therapies slow the progression of the tumor, or substantially delay or prevent its onset, or reduce or prevent the incidence of tumor metastasis and/or recurrance.

A TNBCMARKER includes for example FANCD2, XPF, pMK2, PAR, PARP1, MLH1, ATM, RAD51, BRCA1, ERCC1, NQO1, p53, Ki67. One, two, three, four, five, ten or more TNBCMARKERS are measured. Preferably, at least two TNBCMARKERS selected from FANCD2, XPF, pMK2, PAR, PARP1, MLH1, ATM, RAD51, BRCA1, and ERCC1, are measured. In some aspects FANDC2, BRCA1, or RAD51 and at least one TNBCMARKER selected from XPF or ERCC1; pMK2 or ATM; or PAR or PARP1 is measured.

In a further aspect the TNBCMARKERS are DNA repair proteins belonging to different DNA repair pathways. Alternatively three or more TNBCMARKERS are measures where TNBCMARKERS belonging to two, three, four, five or more different DNA repair pathways.

In other aspects of the invention FAND2 and at least one TNBCMARKER selected from XPF, pMK2, PAR, PARP1, MLH1, ATM, RAD51, BRCA1, and ERCC1 is measured; XPF and at least one TNBCMARKER selected from FANCD2, pMK2, PAR, PARP1, MLH1, ATM, RAD51, BRCA1, and ERCC1 is measured; pMK2 and at least one TNBCMARKER selected from FANCD2, XPF, PAR, PARP1, MLH1, ATM, RAD51, BRCA1, and ERCC1 is measured; PAR and at least one TNBCMARKER selected from FANCD2, XPF, pMK2, PARP1, MLH1, ATM, RAD51, BRCA1, and ERCC1 is measured; PARP1 and at least one TNBCMARKER selected from FANCD2, XPF, pMK2, PAR, MLH1, ATM, RAD51, BRCA1, and ERCC1; MLH1 and at least one TNBCMARKER selected from FANCD2, XPF, pMK2, PAR, PARP1, ATM, RAD51, BRCA1, and ERCC1 is measured; ATM and at least one TNBCMARKER selected from FANCD2, XPF, pMK2, PAR, PARP1, MLH1, RAD51, BRCA1, and ERCC1 is measured; RAD51 and at least one TNBCMARKER selected from FANCD2, XPF, pMK2, PAR, PARP1, MLH1, ATM, BRCA1, and ERCC1 is measured; BRCA1 and at least one FANCD2, XPF, pMK2, PAR, PARP1, MLH1, ATM, RAD51, and ERCC1 is measured; or ERCC1 and at least one FANCD2, XPF, pMK2, PAR, PARP1, MLH1, ATM, RAD51, and BRCA1 is measured. Optionally one or more TNBCMARKERS selected from NQO1, p53, and Ki67 is additionally measured.

### DEFINITIONS

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarker can also include mutated proteins or mutated nucleic acids. Biomarker also encompass non-blood borne factors or non-analyte physiological markers of health status, such as “clinical parameters” defined herein, as well as “traditional laboratory risk factors”, also defined herein. Biomarkers also include any calculated indices created mathematically or combinations of any one or more of the foregoing measurements, including temporal trends and differences. Where available, and unless otherwise described herein, biomarkers which are gene products are identified based on the official letter abbreviation or gene symbol assigned by the international Human Genome Organization Naming Committee (HGNC) and listed at the date of this filing at the US National Center for Biotechnology Information (NCBI) web site (http://www.ncbi.nlm.nih.gov/sites/entrez?db=gene), also known as Entrez Gene.

“TNBCMARKER” OR “TNBCMAERKER” encompass one or more of all nucleic acids or polypeptides whose levels are changed in subjects who have a triple negative breast cancer or are predisposed to developing a triple negative breast cancer, or at risk of triple negative breast cancer. As used herein TNBCMARKERS includes p53, Ki67, NQO1, XPF, pMK2, PAR, PARP1, MLH1, ERCC1, BRCA1, RAD51, ATM or FANCD2. Individual TNBCMARKERS are collectively referred to herein as, inter alia, “triple negative breast cancer-associated proteins”, “TNBCMARKER polypeptides”, or “TNBCMARKER proteins”. The corresponding nucleic acids encoding the polypeptides are referred to as “triple negative breast cancer-associated nucleic acids”, “triple negative breast cancer-associated genes”, “TNBCMARKER nucleic acids”, or “TNBCMARKER genes”. Unless indicated otherwise, “TNBCMARKER”, “triple negative breast cancer-associated proteins”, “triple negative breast cancer-associated nucleic acids” are meant to refer to any of the biomarkers disclosed herein, e.g p53, Ki67, NQO1, XPF, pMK2, PAR, PARP1, MLH1, ERCC1, BRCA1, RAD51, ATM or FANCD2. The corresponding metabolites of the TNBCMARKER proteins or nucleic acids can also be measured, as well as any of the aforementioned traditional risk marker metabolites.

Physiological markers of health status (e.g., such as age, family history, and other measurements commonly used as traditional risk factors) are referred to as “TNBCMARKER physiology”. Calculated indices created from mathematically combining measurements of one or more, preferably two or more of the aforementioned classes of TNBCMARKER S are referred to as “TNBCMARKER indices”.

A “Clinical indicator” is any physiological datum used alone or in conjunction with other data in evaluating the physiological condition of a collection of cells or of an organism. This term includes pre-clinical indicators.

“Clinical parameters” encompasses all non-sample or non-analyte biomarkers of subject health status or other characteristics, such as, without limitation, age (Age), ethnicity (RACE), gender (Sex), or family history (FamHX).

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Of particular use in combining TNBCMARKERS and other biomarkers are linear and non-linear equations and statistical classification analyses to determine the relationship between levels of TNBCMARKERS detected in a subject sample and the subject's risk of disease. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a TNBCMARKER selection technique, such as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcome's expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

“Negative predictive value” or “NPV” is calculated by TN/(TN+FN) or the true negative fraction of all negative test results. It also is inherently impacted by the prevalence of the disease and pre-test probability of the population intended to be tested.

See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4th edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

Finally, hazard ratios and absolute and relative risk ratios within subject cohorts defined by a test are a further measurement of clinical accuracy and utility. Multiple methods are frequently used to defining abnormal or disease values, including reference limits, discrimination limits, and risk thresholds.

“Analytical accuracy” refers to the reproducibility and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Positive predictive value” or “PPV” is calculated by TP/(TP+FP) or the true positive fraction of all positive test results. It is inherently impacted by the prevalence of the disease and pre-test probability of the population intended to be tested.

“Risk” in the context of the present invention, relates to the probability that an event will occur over a specific time period, as in the conversion to a recurrent cancer, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion.

“Risk evaluation,” or “evaluation of risk” in the context of the present invention encompasses making a prediction of the probability, odds, or likelihood that an event or disease state may occur, the rate of occurrence of the event or conversion from one disease state to another, i.e., from a primary tumor to a metastatic tumor or to one at risk of developing a metastatic, or from at risk of a primary metastatic event to a more secondary metastatic event or to the coversion of a state of remission to a recurrence of the cancer. Risk evaluation can also comprise prediction of future clinical parameters, traditional laboratory risk factor values, or other indices of cancer, either in absolute or relative terms in reference to a previously measured population. The methods of the present invention may be used to make continuous or categorical measurements of the risk of cancer recurrance thus diagnosing and defining the risk spectrum of a category of subjects defined as being at risk for cancer recurrance. In the categorical scenario, the invention can be used to discriminate between normal and other subject cohorts at higher risk for cancer recurrance. Such differing use may require different TNBCMARKER combinations and individualized panels, mathematical algorithms, and/or cut-off points, but be subject to the same aforementioned measurements of accuracy and performance for the respective intended use.

A “sample” in the context of the present invention is a biological sample isolated from a subject and can include, by way of example and not limitation, tissue biopies, whole blood, serum, plasma, blood cells, endothelial cells, lymphatic fluid, ascites fluid, interstitital fluid (also known as “extracellular fluid” and encompasses the fluid found in spaces between cells, including, inter alia, gingival crevicular fluid), bone marrow, cerebrospinal fluid (CSF), saliva, mucous, sputum, sweat, urine, or any other secretion, excretion, or other bodily fluids.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. The p-values is a measure of probability that a difference between groups during an experiment happened by chance. (P(z≧z_{observed})). For example, a p-value of 0.01 means that there is a 1 in 100 chance the result occurred by chance. The lower the p-value, the more likely it is that the difference between groups was caused by treatment. An alteration is statistically significant if the p-value is at least 0.05. Preferably, the p-value is 0.04, 0.03, 0.02, 0.01, 0.005, 0.001 or less.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of tumor recurrence. A subject can be male or female. A subject can be one who has been previously diagnosed or identified as having primary tumor, a recurrent tumor or a metastatic tumor, and optionally has already undergone, or is undergoing, a therapeutic intervention for the tumor. Alternatively, a subject can also be one who has not been previously diagnosed as having a recurrent tumor. For example, a subject can be one who exhibits one or more risk factors for a recurrent tumor.

“TN” is true negative, which for a disease state test means classifying a non-disease or normal subject correctly.

“TP” is true positive, which for a disease state test means correctly classifying a disease subject.

“Traditional laboratory risk factors” correspond to biomarkers isolated or derived from subject samples and which are currently evaluated in the clinical laboratory and used in traditional global risk assessment algorithms. Traditional laboratory risk factors for tumor recurrence s include for example [ADD] Proliferative index, tumor-infiltrating lymphocytes. Other traditional laboratory risk factors for tumor recurrence known to those skilled in the art.

Methods and Uses of the Invention

The methods disclosed herein are used with subjects at risk for developing a recoccurance of triple negative breast cancer, subjects who may or may not have already been diagnosed with triple negative breast cancer and subjects undergoing treatment and/or therapies for a triple negative breast cancer. The methods of the present invention can also be used to monitor or select a treatment regimen for a subject who has a triple negative breast cancer, and to screen subjects who have not been previously diagnosed as having a triple negative breast cancer. Treatment regimens include for example but not limited to anthracylines, anti-metabolites such as methotrexate, radiation, taxols, platinums, and combinations of thereof.

Preferably, the methods of the present invention are used to identify and/or diagnose subjects who are asymptomatic for a cancer recurrence. “Asymptomatic” means not exhibiting the traditional symptoms.

The methods of the present invention may also used to identify and/or diagnose subjects already at higher risk of developing a cancer recurrence or based on solely on the traditional risk factors.

A subject having a triple negative breast cancer recurrence can be identified by measuring the amounts (including the presence or absence) of an effective number of TNBCMARKERS in a subject-derived sample and the amounts are then compared to a reference value. Alterations in the amounts and patterns of expression of biomarkers, such as proteins, polypeptides, nucleic acids and polynucleotides, polymorphisms of proteins, polypeptides, nucleic acids, and polynucleotides, mutated proteins, polypeptides, nucleic acids, and polynucleotides, or alterations in the molecular quantities of metabolites or other analytes in the subject sample compared to the reference value are then identified. By an effective number is meant the number of constituents that need to be measured in order to directly predict the cancer recurrence in a subject having triple negative breast cancer. Preferably the constituents are selected as to predict cancer recurrence with least 75% accuracy, more preferably 80%, 85%, 90%, 95%, 97%, 98%, 99% or greater accuracy.

A reference value can be relative to a number or value derived from population studies, including without limitation, such subjects having the same cancer, subject having the same or similar age range, subjects in the same or similar ethnic group, subjects having family histories of cancer, or relative to the starting sample of a subject undergoing treatment for a cancer. Such reference values can be derived from statistical analyses and/or risk prediction data of populations obtained from mathematical algorithms and computed indices of cancer recurrence. Reference TNBCMARKER indices can also be constructed and used using algorithms and other methods of statistical and structural classification.

In one embodiment of the present invention, the reference value is the amount of TNBCMARKERS in a control sample derived from one or more subjects who are not at risk or at low risk for developing a recurrence of a triple negative breast cancer. In another embodiment of the present invention, the reference value is the amount of TNBCMARKERS in a control sample derived from one or more subjects who are asymptomatic and/or lack traditional risk factors triple negative breast cancer. In a further embodiment, such subjects are monitored and/or periodically retested for a diagnostically relevant period of time (“longitudinal studies”) following such test to verify continued absence of a triple negative breast cancer (disease or event free survival). Such period of time may be one year, two years, two to five years, five years, five to ten years, ten years, or ten or more years from the initial testing date for determination of the reference value. Furthermore, retrospective measurement of TNBCMARKERS in properly banked historical subject samples may be used in establishing these reference values, thus shortening the study time required.

A reference value can also comprise the amounts of TNBCMARKERS derived from subjects who show an improvement in risk factors as a result of treatments and/or therapies for the cancer. A reference value can also comprise the amounts of TNBCMARKERS derived from subjects who have confirmed disease by known invasive or non-invasive techniques, or are at high risk for developing triple negative breast cancer, or who have suffered from triple negative breast cancer.

In another embodiment, the reference value is an index value or a baseline value. An index value or baseline value is a composite sample of an effective amount of TNBCMARKERS from one or more subjects who do not have a triple negative breast cancer or subjects who are asymptomatic a triple negative breast cancer. A baseline value can also comprise the amounts of TNBCMARKERS in a sample derived from a subject who has shown an improvement in triple negative breast cancer risk factors as a result of cancer treatments or therapies. In this embodiment, to make comparisons to the subject-derived sample, the amounts of TNBCMARKERS are similarly calculated and compared to the index value. Optionally, subjects identified as having triple negative breast cancer, or being at increased risk of developing a triple negative breast cancer are chosen to receive a therapeutic regimen to slow the progression the cancer, or decrease or prevent the risk of developing a triple negative breast cancer.

The progression of a triple negative breast cancer, or effectiveness of a cancer treatment regimen can be monitored by detecting a TNBCMARKER in an effective amount (which may be two or more) of samples obtained from a subject over time and comparing the amount of TNBCMARKERS detected. For example, a first sample can be obtained prior to the subject receiving treatment and one or more subsequent samples are taken after or during treatment of the subject. The cancer is considered to be progressive (or, alternatively, the treatment does not prevent progression) if the amount of TNBCMARKER changes over time relative to the reference value, whereas the cancer is not progressive if the amount of TNBCMARKERS remains constant over time (relative to the reference population, or “constant” as used herein). The term “constant” as used in the context of the present invention is construed to include changes over time with respect to the reference value.

Additionally, therapeutic or prophylactic agents suitable for administration to a particular subject can be identified by detecting one or more of the TNBCMARKERS in an effective amount (which may be two or more) in a sample obtained from a subject, exposing the subject-derived sample to a test compound that determines the amount (which may be two or more) of TNBCMARKERS in the subject-derived sample. Accordingly, treatments or therapeutic regimens for use in subjects having a cancer, or subjects at risk for developing triple negative breast cancer or a recurrence or triple negative breast can be selected based on the amounts of TNBCMARKERS in samples obtained from the subjects and compared to a reference value. Two or more treatments or therapeutic regimens can be evaluated in parallel to determine which treatment or therapeutic regimen would be the most efficacious for use in a subject to delay onset, or slow progression of the cancer.

The present invention further provides a method for screening for changes in marker expression associated with triple negative breast cancer, by determining one or more of the TNBCMARKERS in a subject-derived sample, comparing the amounts of the TNBCMARKERS in a reference sample, and identifying alterations in amounts in the subject sample compared to the reference sample.

If the reference sample, e.g., a control sample, is from a subject that does not have a triple negative breast cancer, or if the reference sample reflects a value that is relative to a person that has a high likelihood of rapid progression to a recurrence of triple negative breast cancer, a similarity in the amount of the TNBCMARKER in the test sample and the reference sample indicates that the treatment is efficacious. However, a difference in the amount of the TNBCMARKER in the test sample and the reference sample indicates a less favorable clinical outcome or prognosis.

By “efficacious”, it is meant that the treatment leads to a decrease in the amount or activity of a TNBCMARKER protein, nucleic acid, polymorphism, metabolite, or other analyte. Assessment of the risk factors disclosed herein can be achieved using standard clinical protocols. Efficacy can be determined in association with any known method for diagnosing, identifying, or treating a triple negative breast cancer.

The present invention also comprises a kit with a detection reagent that binds to two or more of the TNBCMARKERS proteins, nucleic acids, polymorphisms, metabolites, or other analytes. Also provided by the invention is an array of detection reagents, e.g., antibodies and/or oligonucleotides that can bind to two or more TNBCMARKER proteins or nucleic acids, respectively.

Also provided by the present invention is a method for treating one or more subjects at risk for developing a triple negative breast cancer recurrence by detecting the presence of altered amounts of an effective amount of the TNBCMARKERS present in a sample from the one or more subjects; and treating the one or more subjects with one or more cancer-modulating drugs until altered amounts or activity of the TNBCMARKERS return to a baseline value measured in one or more subjects at low risk for developing a metastatic disease, or alternatively, in subjects who do not exhibit any of the traditional risk factors formetastatic disease.

Also provided by the present invention is a method for treating one or more subjects having triple negative breast cancer by detecting the presence of altered levels of an effective amount of the TNBCMARKERS present in a sample from the one or more subjects; and treating the one or more subjects with one or more cancer-modulating drugs until altered amounts or activity of the TNBCMARKERS return to a baseline value measured in one or more subjects at low risk for developing cancer recurrance.

Also provided by the present invention is a method for evaluating changes in the risk of developing a triple negative breast cancer recurrence in a subject diagnosed with cancer, by detecting an effective amount of the TNBCMARKERS (which may be two or more) in a first sample from the subject at a first period of time, detecting the amounts of the TNBCMARKERS in a second sample from the subject at a second period of time, and comparing the amounts of the TNBCMARKERS detected at the first and second periods of time.

Diagnostic and Prognostic Indications of the Invention

The invention allows the diagnosis and prognosis of triple negative breast cancer. The risk of developing triple negative breast cancer of a recurrence or triple negative breast cancer can be detected by measuring an effective amount of the TNBCMARKER proteins, nucleic acids, polymorphisms, metabolites, and other analytes (which may be two or more) in a test sample (e.g., a subject derived sample), and comparing the effective amounts to reference or index values, often utilizing mathematical algorithms or formula in order to combine information from results of multiple individual TNBCMARKERS and from non-analyte clinical parameters into a single measurement or index. Subjects identified as having an increased risk of triple negative breast cancer can optionally be selected to receive treatment regimens, such as administration of prophylactic or therapeutic compounds to prevent or delay the onset of a triple negative breast cancer or a reoccurrence of triple negative breast cancer.

The amount of the TNBCMARKER protein, nucleic acid, polymorphism, metabolite, or other analyte can be measured in a test sample and compared to the “normal control level,” utilizing techniques such as reference limits, discrimination limits, or risk defining thresholds to define cutoff points and abnormal values. The “normal control level” means the level of one or more TNBCMARKERS or combined TNBCMARKER indices typically found in a subject not suffering from triple negative breast cancer. Such normal control level and cutoff points may vary based on whether a TNBCMARKER is used alone or in a formula combining with other TNBCMARKERS into an index. Alternatively, the normal control level can be a database of TNBCMARKER patterns from previously tested subjects who did not develop a recurrence or triple negative breast cancer over a clinically relevant time horizon.

The present invention may be used to make continuous or categorical measurements of the risk of conversion to at triple negative breast cancer recurrence, thus diagnosing and defining the risk spectrum of a category of subjects defined as at risk for having a cancer recurrence. In the categorical scenario, the methods of the present invention can be used to discriminate between normal and disease subject cohorts. In other embodiments, the present invention may be used so as to discriminate those at risk for having cancer recurrence from those having more rapidly progressing (or alternatively those with a shorter probable time horizon to cancer recurrence) to a cancer reoccurrance from those more slowly progressing (or with a longer time horizon to a cancer reoccurrance), or those having cancer reoccurrance from normal. Such differing use may require different TNBCMARKER combinations in individual panel, mathematical algorithm, and/or cut-off points, but be subject to the same aforementioned measurements of accuracy and other performance metrics relevant for the intended use.

Identifying the subject at risk of having a triple negative breast cancer recurrence enables the selection and initiation of various therapeutic interventions or treatment regimens in order to delay, reduce or prevent that subject's conversion to a cancer recurrence. Levels of an effective amount of TNBCMARKER proteins, nucleic acids, polymorphisms, metabolites, or other analytes also allows for the course of treatment of triple negative breast cancer or cancer reccurrence to be monitored. In this method, a biological sample can be provided from a subject undergoing treatment regimens, e.g., drug treatments, for cancer. If desired, biological samples are obtained from the subject at various time points before, during, or after treatment.

By virtue of TNBCMARKERs' being functionally active, by elucidating its function, subjects with high TNBCMARKERs, for example, can be managed with agents/drugs that preferentially target such pathways.

The present invention can also be used to screen patient or subject populations in any number of settings. For example, a health maintenance organization, public health entity or school health program can screen a group of subjects to identify those requiring interventions, as described above, or for the collection of epidemiological data. Insurance companies (e.g., health, life or disability) may screen applicants in the process of determining coverage or pricing, or existing clients for possible intervention. Data collected in such population screens, particularly when tied to any clinical progression to conditions like cancer or cancer reoccurrence, will be of value in the operations of, for example, health maintenance organizations, public health programs and insurance companies. Such data arrays or collections can be stored in machine-readable media and used in any number of health-related data management systems to provide improved healthcare services, cost effective healthcare, improved insurance operation, etc. See, for example, U.S. Patent Application No. 2002/0038227; U.S. Patent Application No. US 2004/0122296; U.S. Patent Application No. US 2004/0122297; and U.S. Pat. No. 5,018,067. Such systems can access the data directly from internal data storage or remotely from one or more data storage sites as further detailed herein.

A machine-readable storage medium can comprise a data storage material encoded with machine readable data or data arrays which, when using a machine programmed with instructions for using said data, is capable of use for a variety of purposes, such as, without limitation, subject information relating to cancer reoccurrance risk factors over time or in response drug therapies. Measurements of effective amounts of the biomarkers of the invention and/or the resulting evaluation of risk from those biomarkers can implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein. The health-related data management system of the invention may also be considered to be implemented as a computer-readable storage medium, configured with a computer program, where the storage medium so configured causes a computer to operate in a specific and predefined manner to perform various functions described herein.

Levels of an effective amount of TNBCMARKER proteins, nucleic acids, polymorphisms, metabolites, or other analytes can then be determined and compared to a reference value, e.g. a control subject or population whose metastatic state is known or an index value or baseline value. The reference sample or index value or baseline value may be taken or derived from one or more subjects who have been exposed to the treatment, or may be taken or derived from one or more subjects who are at low risk of developing cancer or cancer reoccurrance, or may be taken or derived from subjects who have shown improvements in as a result of exposure to treatment. Alternatively, the reference sample or index value or baseline value may be taken or derived from one or more subjects who have not been exposed to the treatment. For example, samples may be collected from subjects who have received initial treatment for cancer or a metastatic event and subsequent treatment for cancer or cancer reoccurrance to monitor the progress of the treatment. A reference value can also comprise a value derived from risk prediction algorithms or computed indices from population studies such as those disclosed herein.

The TNBCMARKERS of the present invention can thus be used to generate a “reference TNBCMARKER profile” of those subjects who do not have triple negative breast cancer or are not at risk of having a triple negative breast cancer reoccurrance, and would not be expected to develop cancer or a cancer reoccurrance. The TNBCMARKERS disclosed herein can also be used to generate a “subject TNBCMARKER profile” taken from subjects who have cancer or are at risk for having a cancer reoccurrance. The subject TNBCMARKER profiles can be compared to a reference TNBCMARKER profile to diagnose or identify subjects at risk for developing cancer or a cancer reoccurrance, to monitor the progression of disease, as well as the rate of progression of disease, and to monitor the effectiveness of treatment modalities. The reference and subject TNBCMARKER profiles of the present invention can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history. The machine-readable media can also contain information relating to other disease-risk algorithms and computed indices such as those described herein.

Differences in the genetic makeup of subjects can result in differences in their relative abilities to metabolize various drugs, which may modulate the symptoms or risk factors of cancer or cancer reoccurrence. Subjects that have cancer, or at risk for developing cancer or a cancer reoccurrance t can vary in age, ethnicity, and other parameters. Accordingly, use of the TNBCMARKERS disclosed herein, both alone and together in combination with known genetic factors for drug metabolism, allow for a pre-determined level of predictability that a putative therapeutic or prophylactic to be tested in a selected subject will be suitable for treating or preventing cancer or a cancer reoccurrance in the subject.

To identify therapeutics or drugs that are appropriate for a specific subject, a test sample from the subject can also be exposed to a therapeutic agent or a drug, and the level of one or more of TNBCMARKER proteins, nucleic acids, polymorphisms, metabolites or other analytes can be determined. The level of one or more TNBCMARKERS can be compared to sample derived from the subject before and after treatment or exposure to a therapeutic agent or a drug, or can be compared to samples derived from one or more subjects who have shown improvements in risk factors (e.g., clinical parameters or traditional laboratory risk factors) as a result of such treatment or exposure.

A subject cell (i.e., a cell isolated from a subject) can be incubated in the presence of a candidate agent and the pattern of TNBCMARKER expression in the test sample is measured and compared to a reference profile, e.g., a metastatic disease reference expression profile or a non-disease reference expression profile or an index value or baseline value. The test agent can be any compound or composition or combination thereof, including, dietary supplements. For example, the test agents are agents frequently used in cancer treatment regimens and are described herein.

The aforementioned methods of the invention can be used to evaluate or monitor the progression and/or improvement of subjects who have been diagnosed with a cancer, and who have undergone surgical interventions.

Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between subjects having cancer, or at risk for triple negative breast cancer or a triple negative breast cancer reoccurrance, is based on whether the subjects have an “effective amount” or a “significant alteration” in the levels of a TNBCMARKER. By “effective amount” or “significant alteration,” it is meant that the measurement of an appropriate number of TNBCMARKERS (which may be one or more) is different than the predetermined cut-off point (or threshold value) for that TNBCMARKER(S) and therefore indicates that the subject has cancer or is at risk for having a metastatic event for which the TNBCMARKER(S) is a TNBCMARKER. The difference in the level of TNBCMARKER between normal and abnormal is preferably statistically significant. As noted below, and without any limitation of the invention, achieving statistical significance, and thus the preferred analytical and clinical accuracy, generally but not always requires that combinations of several TNBCMARKERS be used together in panels and combined with mathematical algorithms in order to achieve a statistically significant TNBCMARKER index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic accuracy”, is herein defined as a test or assay (such as the test of the invention for determining the clinically significant presence of TNBCMARKERS, which thereby indicates the presence of cancer and/or a risk of having a cancer recurrance) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing cancer or metastatic event, and the bottom quartile comprising the group of subjects having the lowest relative risk for developing cancer or a metastatic event. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for each quartile remain clinically useful are widely used as risk factors for a disease; such is the case with total cholesterol and for many inflammatory biomarkers with respect to their prediction of future metastatic events. Often such lower diagnostic accuracy tests must be combined with additional parameters in order to derive meaningful clinical thresholds for therapeutic intervention, as is done with the aforementioned global risk assessment indices.

A health economic utility function is an yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category (such as those atirisk for having a cancer reoccurrence) has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the TNBCMARKERS of the invention allows for one of skill in the art to use the TNBCMARKERS to identify, diagnose, or prognose subjects with a pre-determined level of predictability and performance.

Construction of Clinical Algorithms

Any formula may be used to combine TNBCMARKER results into indices useful in the practice of the invention. As indicated above, and without limitation, such indices may indicate, among the various other indications, the probability, likelihood, absolute or relative risk, time to or rate of conversion from one to another disease states, or make predictions of future biomarker measurements of metastatic disease. This may be for a specific time period or horizon, or for remaining lifetime risk, or simply be provided as an index relative to another reference subject population.

Although various preferred formula are described here, several other model and formula types beyond those mentioned herein and in the definitions above are well known to one skilled in the art. The actual model type or formula used may itself be selected from the field of potential models based on the performance and diagnostic accuracy characteristics of its results in a training population. The specifics of the formula itself may commonly be derived from TNBCMARKER results in the relevant training population. Amongst other uses, such formula may be intended to map the feature space derived from one or more TNBCMARKER inputs to a set of subject classes (e.g. useful in predicting class membership of subjects as normal, at risk for having a metastatic event, having cancer), to derive an estimation of a probability function of risk using a Bayesian approach (e.g. the risk of cancer or a metastatic event), or to estimate the class-conditional probabilities, then use Bayes' rule to produce the class probability function as in the previous case.

Preferred formulas include the broad class of statistical classification algorithms, and in particular the use of discriminant analysis. The goal of discriminant analysis is to predict class membership from a previously identified set of features. In the case of linear discriminant analysis (LDA), the linear combination of features is identified that maximizes the separation among groups by some criteria. Features can be identified for LDA using an eigengene based approach with different thresholds (ELDA) or a stepping algorithm based on a multivariate analysis of variance (MANOVA). Forward, backward, and stepwise algorithms can be performed that minimize the probability of no separation based on the Hotelling-Lawley statistic.

Eigengene-based Linear Discriminant Analysis (ELDA) is a feature selection technique developed by Shen et al. (2006). The formula selects features (e.g. biomarkers) in a multivariate framework using a modified eigen analysis to identify features associated with the most important eigenvectors. “Important” is defined as those eigenvectors that explain the most variance in the differences among samples that are trying to be classified relative to some threshold.

A support vector machine (SVM) is a classification formula that attempts to find a hyperplane that separates two classes. This hyperplane contains support vectors, data points that are exactly the margin distance away from the hyperplane. In the likely event that no separating hyperplane exists in the current dimensions of the data, the dimensionality is expanded greatly by projecting the data into larger dimensions by taking non-linear functions of the original variables (Venables and Ripley, 2002). Although not required, filtering of features for SVM often improves prediction. Features (e.g., biomarkers) can be identified for a support vector machine using a non-parametric Kiruskal-Wallis (KW) test to select the best univariate features. A random forest (RF, Breiman, 2001) or recursive partitioning (RPART, Breiman et al., 1984) can also be used separately or in combination to identify biomarker combinations that are most important. Both KW and RF require that a number of features be selected from the total. RPART creates a single classification tree using a subset of available biomarkers.

Other formula may be used in order to pre-process the results of individual TNBCMARKER measurement into more valuable forms of information, prior to their presentation to the predictive formula. Most notably, normalization of biomarker results, using either common mathematical transformations such as logarithmic or logistic functions, as normal or other distribution positions, in reference to a population's mean values, etc. are all well known to those skilled in the art. Of particular interest are a set of normalizations based on Clinical Parameters such as age, gender, race, or sex, where specific formula are used solely on subjects within a class or continuously combining a Clinical Parameter as an input. In other cases, analyte-based biomarkers can be combined into calculated variables which are subsequently presented to a formula.

In addition to the individual parameter values of one subject potentially being normalized, an overall predictive formula for all subjects, or any known class of subjects, may itself be recalibrated or otherwise adjusted based on adjustment for a population's expected prevalence and mean biomarker parameter values, according to the technique outlined in D'Agostino et al, (2001) JAMA 286:180-187, or other similar normalization and recalibration techniques. Such epidemiological adjustment statistics may be captured, confirmed, improved and updated continuously through a registry of past data presented to the model, which may be machine readable or otherwise, or occasionally through the retrospective query of stored samples or reference to historical studies of such parameters and statistics. Additional examples that may be the subject of formula recalibration or other adjustments include statistics used in studies by Pepe, M. S. et al, 2004 on the limitations of odds ratios; Cook, N. R., 2007 relating to ROC curves. Finally, the numeric result of a classifier formula itself may be transformed post-processing by its reference to an actual clinical population and study results and observed endpoints, in order to calibrate to absolute risk and provide confidence intervals for varying numeric results of the classifier or risk formula. An example of this is the presentation of absolute risk, and confidence intervals for that risk, derivied using an actual clinical study, chosen with reference to the output of the recurrence score formula in the Oncotype Dx product of Genomic Health, Inc. (Redwood City, Calif.). A further modification is to adjust for smaller sub-populations of the study based on the output of the classifier or risk formula and defined and selected by their Clinical Parameters, such as age or sex.

Combination with Clinical Parameters and Traditional Laboratory Risk Factors

Any of the aforementioned Clinical Parameters may be used in the practice of the invention as a TNBCMARKER input to a formula or as a pre-selection criteria defining a relevant population to be measured using a particular TNBCMARKER panel and formula. As noted above, Clinical Parameters may also be useful in the biomarker normalization and pre-processing, or in TNBCMARKER selection, panel construction, formula type selection and derivation, and formula result post-processing. A similar approach can be taken with the Traditional Laboratory Risk Factors, as either an input to a formula or as a pre-selection criterium.

Measurement of TNBCMARKERS

The actual measurement of levels or amounts of the TNBCMARKERS can be determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, amounts of TNBCMARKERS can be measured using reverse-transcription-based PCR assays (RT-PCR), e.g., using primers specific for the differentially expressed sequence of genes or by branch-chain RNA amplification and detection methods by Panomics, Inc. Amounts of TNBCMARKERS can also be determined at the protein level, e.g., by measuring the levels of peptides encoded by the gene products described herein, or subcellular localization or activities thereof using technological platform such as for example AQUA. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed.

The TNBCMARKER proteins, polypeptides, mutations, and polymorphisms thereof can be detected in any suitable manner, but is typically detected by contacting a sample from the subject with an antibody which binds the TNBCMARKER protein, polypeptide, mutation, or polymorphism and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Immunoassays carried out in accordance with the present invention may be homogeneous assays or heterogeneous assays. In a homogeneous assay the immunological reaction usually involves the specific antibody (e.g., anti-TNBCMARKER protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, immunoprecipitation, quantum dots, multiplex fluorochromes, chemiluminescence methods, electrochemiluminescence (ECL) or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹), enzyme labels (e.g., horseradish peroxidase, alkaline phosphatase), and fluorescent labels (e.g., fluorescein, Alexa, green fluorescent protein, rhodamine) in accordance with known techniques. Highly sensitivity antibody detection strategies may be used that allow for evaluation of the antigen-antibody binding in a non-amplified configuration. In addition, antibodies may be conjugated to oligonucleotides, and followed by Polymerase Chain Reaction and a variety of oligonucleotide detection methods.

Antibodies can also be useful for detecting post-translational modifications of TNBCMARKER proteins, polypeptides, mutations, and polymorphisms, such as tyrosine phosphorylation, threonine phosphorylation, serine phosphorylation, glycosylation (e.g., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF) (Wirth, U. et al. (2002) Proteomics 2(10): 1445-51). In addition to post-translation modifications, these processes may be coupled to localization of the protein, such that a re-localization process is monitored, and the biomarker is evaluated in a relative fashion exhibited by the constancy or change to the ratio of the protein in different compartments. Important to several of the proteins in TNBCMARKERs, nuclear, nuclear foci, and cytoplasmic sites in tumor cells are evident.

For TNBCMARKER proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

Using sequence information provided by the database entries for the TNBCMARKER sequences, expression of the TNBCMARKER sequences can be detected (if present) and measured using techniques well known to one of ordinary skill in the art. For example, sequences within the sequence database entries corresponding to TNBCMARKER sequences, or within the sequences disclosed herein, can be used to construct probes for detecting TNBCMARKER RNA sequences in, e.g., Northern blot hybridization analyses or methods which specifically, and, preferably, quantitatively amplify specific nucleic acid sequences. As another example, the sequences can be used to construct primers for specifically amplifying the TNBCMARKER sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), e.g., using primers specific for the differentially expressed sequences. RNA can also be quantified using, for example, other target amplification methods (e.g., TMA, SDA, NASBA), or signal amplification methods (e.g., bDNA), and the like.

Alternatively, TNBCMARKER protein and nucleic acid metabolites can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. (See, WO 04/056456 and WO 04/088309, each of which are hereby incorporated by reference in their entireties) In this regard, other TNBCMARKER analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others. Other TNBCMARKER metabolites can be similarly detected using reagents that are specifically designed or tailored to detect such metabolites.

Kits

The invention also includes a TNBCMARKER-detection reagent, e.g., nucleic acids that specifically identify one or more TNBCMARKER nucleic acids by having homologous nucleic acid sequences, such as oligonucleotide sequences, complementary to a portion of the TNBCMARKER nucleic acids or antibodies to proteins encoded by the TNBCMARKER nucleic acids packaged together in the form of a kit. The oligonucleotides can be fragments of the TNBCMARKER genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a Northern hybridization or a sandwich ELISA as known in the art.

For example, TNBCMARKER detection reagents can be immobilized on a solid matrix such as a porous strip to form at least one TNBCMARKER detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of TNBCMARKERS present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by TNBCMARKERS. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.).

Suitable sources for antibodies for the detection of TNBCMARKERS include commercially available sources such as, for example, Abazyme, Abnova, Affinity Biologicals, Antibody Shop, Biogenesis, Biosense Laboratories, Calbiochem, Cell Sciences, Chemicon International, Chemokine, Clontech, Cytolab, DAKO, Diagnostic BioSystems, eBioscience, Endocrine Technologies, Enzo Biochem, Eurogentec, Fusion Antibodies, Genesis Biotech, GloboZymes, Haematologic Technologies, Immunodetect, Immunodiagnostik, Immunometrics, Immunostar, Immunovision, Biogenex, Invitrogen, Jackson ImmunoResearch Laboratory, KMI Diagnostics, Koma Biotech, LabFrontier Life Science Institute, Lee Laboratories, Lifescreen, Maine Biotechnology Services, Mediclone, MicroPharm Ltd., ModiQuest, Molecular Innovations, Molecular Probes, Neoclone, Neuromics, New England Biolabs, Novocastra, Novus Biologicals, Oncogene Research Products, Orbigen, Oxford Biotechnology, Panvera, PerkinElmer Life Sciences, Pharmingen, Phoenix Pharmaceuticals, Pierce Chemical Company, Polymun Scientific, Polysciences, Inc., Promega Corporation, Proteogenix, Protos Immunoresearch, QED Biosciences, Inc., R&D Systems, Repligen, Research Diagnostics, Roboscreen, Santa Cruz Biotechnology, Seikagaku America, Serological Corporation, Serotec, SigmaAldrich, StemCell Technologies, Synaptic Systems GmbH, Technopharm, Terra Nova Biotechnology, TiterMax, Trillium Diagnostics, Upstate Biotechnology, US Biological, Vector Laboratories, Wako Pure Chemical Industries, and Zeptometrix. However, the skilled artisan can routinely make antibodies, nucleic acid probes, e.g., oligonucleotides, aptamers, siRNAs, antisense oligonucleotides, against any of the TNBCMARKERS disclosed herein.

### EXAMPLES

### Example 1

### General Methods

Patient Cohort

One hundred and forty three previously treated women with triple negative breast cancers were identified and used their archived, formalin-fixed, paraffin-embedded primary excision biopsies to create a tissue microarray (TMA). The majority of these patients were treated with anthracycline-based chemotherapy in the adjuvant setting.

Antibody IHC

The TMA was stained using antibodies against proteins in DNA repair pathways including XPF (nucleotide excision repair), FANCD2 (Fanconi Anemia pathway), MLH1 (mismatch repair), PARP1 (base excision repair), PAR (base excision repair), pMK2 (MapkapKinase2, DNA damage response), P53, and Ki67. The antibodies were obtained from the following sources: XPF (AbCam), FANCD2 and p53 (Santa Cruz), MLH1 and Ki67 (BioCare Medical), PARP1 (AbD Serotec), PAR (poly-ADP ribose, Millipore), phosphoMapkapKinase2 (Cell Signaling Technology). IHC runs were conducted with negative and positive human breast cancer control sections. Tissue sections were deparafinized and rehydrated using standard techniques. Heat-induced epitope retrieval was performed and the tissues were stained with antibody overnight at 4° C. Renaissance TSA™ (Tyramide Signal Amplification) Biotin System (Perkin Elmer) was used for detection of XPF and FANCD2. Super Sensitive™ IHC Detection System (BioGenex) was used for detection of MLH1, PARP1, PAR, pMK2, and Ki67. Envision+System-HRP (Dako) was used for detection of p53. Two-fold antibody dilution ranges were established, and antigen retrieval conditions were set such that antibody was in excess and discriminated between control cancer tissues between low and high expression levels.

Scoring

The stained tissue was evaluated using machine-based image analysis and scoring that incorporated the intensity and quantity of positive tumor nuclei. Scanning and image analysis platforms were from Aperio. Each marker pattern was assessed for quality and by pathology overview. Image analysis algorithms were established for each marker with control breast cancer tumor sections.

Statistical Analysis

Biomarker scoring was correlated with clinical data to assess for correlation with outcome. Patients were randomized into training (60% of patients) and test (40% of patients) cohorts for the development of a multiple marker model. A set of optimal threshold marker values were determined by univariate analysis for each marker that yielded the highest discrimination between Early and Late recurrences. Discriminant and partition analysis was conducted to maximally separate the Training dataset samples into two groups: Early and Late Recurrence. Recurrences are evidence of return of the cancer and are established during patient observation during treatment by clinically accepted criteria. Recurrence time is calculated from the time of diagnosis. In validation exercises, the Training dataset thresholds and marker combinations were applied towards the Test dataset. Kaplan-Meier and Cox proportional hazards were used to evaluate time to recurrence. Statistical outputs for p-value, Apparent Error Rate (AER), Receiver Operator Characteristics and Area Under Curve (AUC), Sensitivity, Specificity, Positive Predictive Power, Negative Predictive Power, Relative Risk (RR), Odds Ratio were computed in the alternative models. With multi-marker models probability tests were conducted to produce AUC values.

### Example 2

### DNA Repair Protein Change is Frequently Observed in Triple Negative Breast Cancer

Breast cancer patients that were diagnosed to have the Triple Negative breast cancer subtype by absence of Her2, ER, and PR by standard histopathology criteria were organized into a study group. The patient biopsies had been obtained from a primary excision biopsy and the patients received chemotherapy according to the approved protocols at the Dana-Farber Cancer Institute. A Tissue Microarray (TMA) containing three 600 m² core regions of cancer tissue per patient was constructed in order to efficiently evaluate the markers, and to minimize the effects of staining variation between patient specimens in immunohistochemistry. The goal of the study was to develop a biomarker pattern at the biopsy stage that would inform how aggressively a patient's tumor would return under standard therapy.

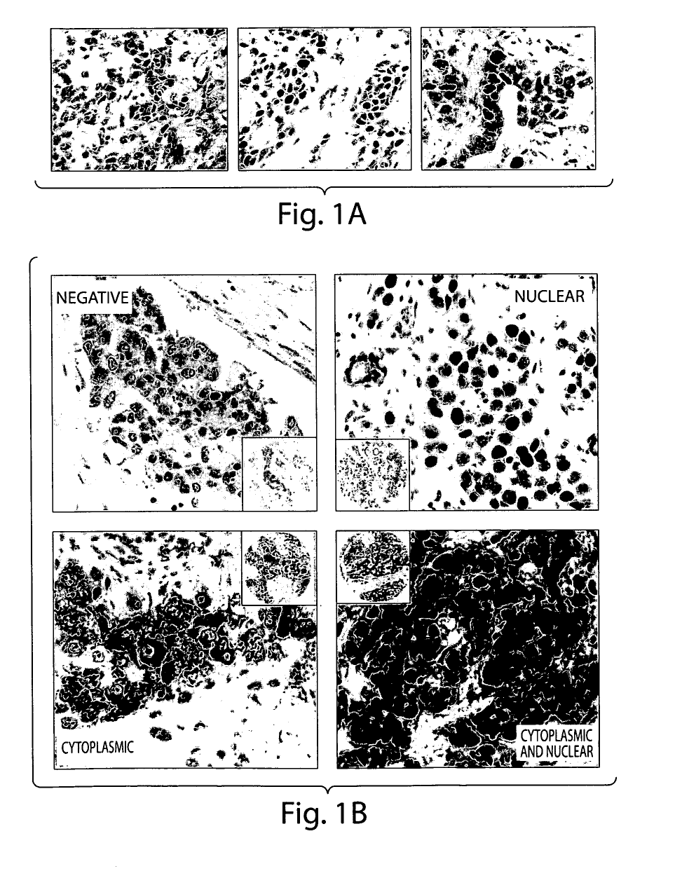

DNA repair pathways are important to the cellular response network to chemotherapy and radiation. In this study, representatives from several of these pathways were investigated for associations with clinical outcome. Ten selected DNA repair protein epitopes, p53, NQO1, and Ki67 proteins were evaluated in serial sections from a triple negative breast cancer TMA. Tumor zones were demarcated per core by pathology review. Expression differences for the markers were quantified by scanning microscope slides into a digital pathology platform (Aperio). Machine-based collection of staining intensities was concentrated to the annotated tumor zones. Marker outputs in 0, 1+, 2+, and 3+ bins were combined in a weighting algorithm to create a relative intensity score from 0-300. For several markers, the intensity of nuclear staining was gauged, in other cases, localization of the marker into different cell compartments was revealed. With the FANCD2 protein pattern, nuclear foci indicative of activation of the Fanconi Anemia core complex and homologous recombination, were observed in some patient biopsies (FIG. 1A). It was found that 19% tumors contained FANCD2 nuclear foci, whereas 23% contained nuclear and cytoplasmic FANCD2. There were 58% of the tumors that were negative for FANCD2 nuclear foci. Likewise, additional post-translational regulation was found in a tumor-specific manner by monitoring the phosphorylation modification of Mapkapkinase2 (pMK2) (FIG. 1B). The pMK2 intracellular location occurred in a distribution of nuclear only, or nuclear+cytoplasmic depending on the tumor. Approximately 10% of the breast cancers contained nuclear staining, 21% had shared cytoplasmic and nuclear staining, and 69% were negative for this activation marker.

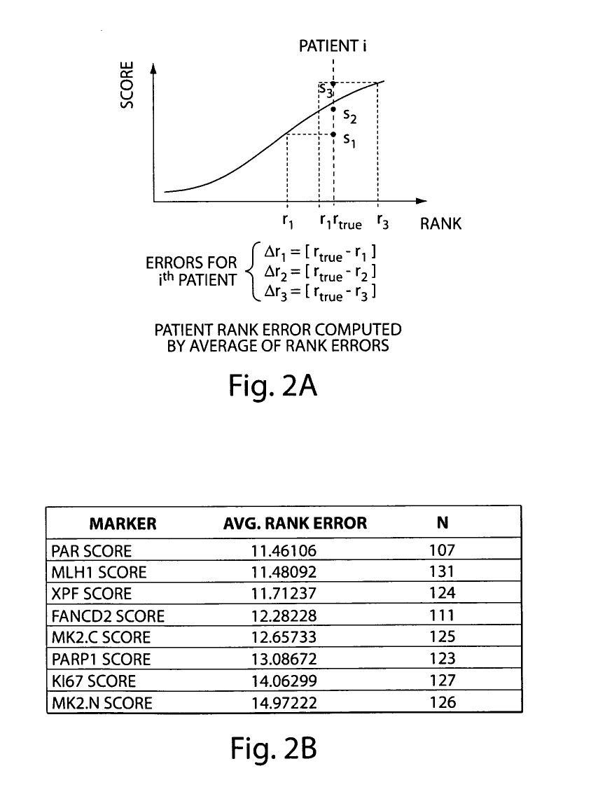

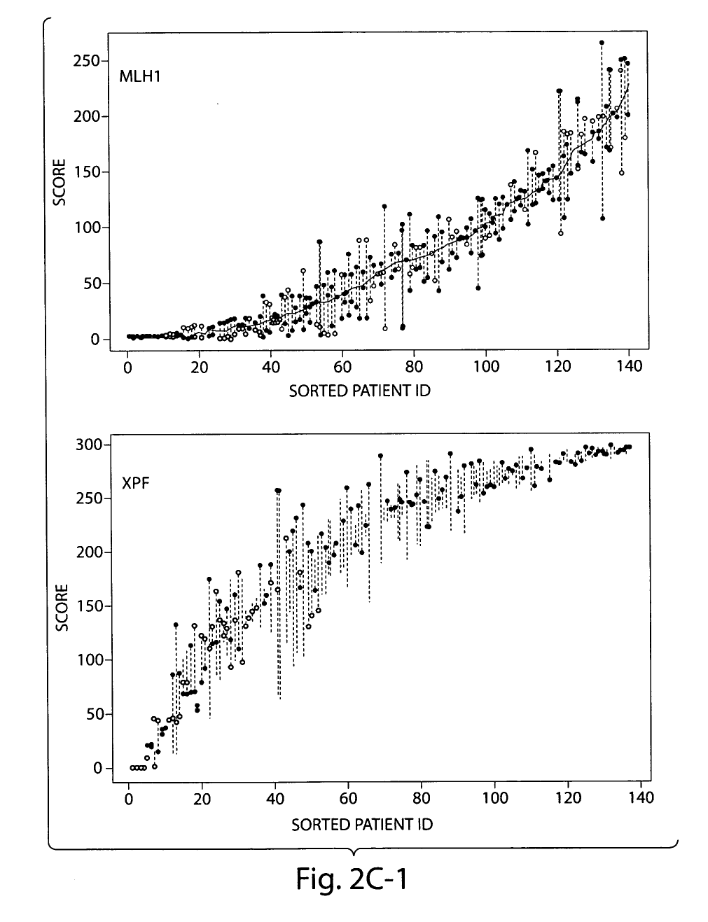

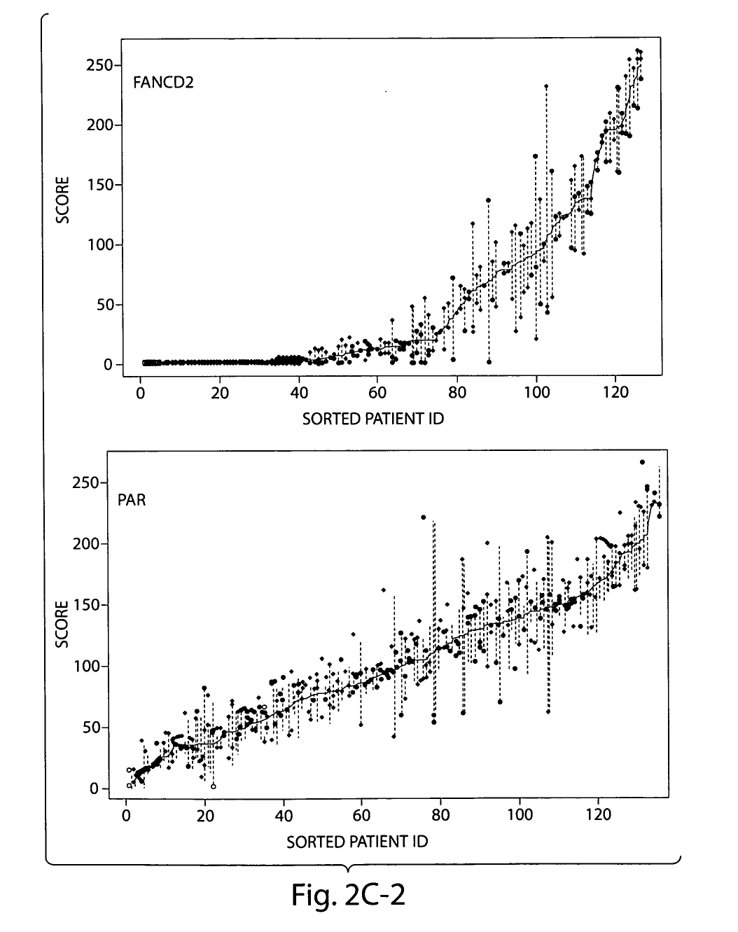

To discriminate the marker output values relative to clinical outcome correlates, it was sought first to resolve whether specimen core-core variation influenced a patient ranking scheme for DNA repair markers. For this purpose, an arbitrary index of patient ranks was established from the lowest values in the cohort to the highest values. The level of variation of each of the markers between triplicate TMA cores was determined, and scored against the patient rank value/marker (FIG. 2A). For the eight DNA repair and proliferation markers tested, it was found that the average rank error was a low percentage of the total (8.8-11.1% DNA Repair, 11.1% Ki67). Therefore, relatively minor variations between triplicate TMA cores do not significantly change the patient rank order for any of the markers tested.

### Example 3

### Association of DNA Repair with Recurrence of Cancer in Chemotherapy-Treated Triple Negative Breast Cancer Patients

Clinical data for 115 patients with primary treatment data was available with a median follow up of 58 months. Median age for the cohort was 49.3 years. Sixty-eight patients were treated with breast conserving therapy and 47 were treated with mastectomy, 17 of which received post mastectomy radiation. One hundred ten patients received chemotherapy as part of their treatment: 42 with anthracycline/cyclophosphamide, 50 with anthracycline/cyclophosphamide/taxane, 15 with cyclophosphamide/methotrexate/5-FU based regimens and 3 other regimens. Eighteen patients had BRCA1 mutations and 5 had unknown variants. There were 37 recurrences: 18 were distant first, 12 were local first and 7 were simultaneous.

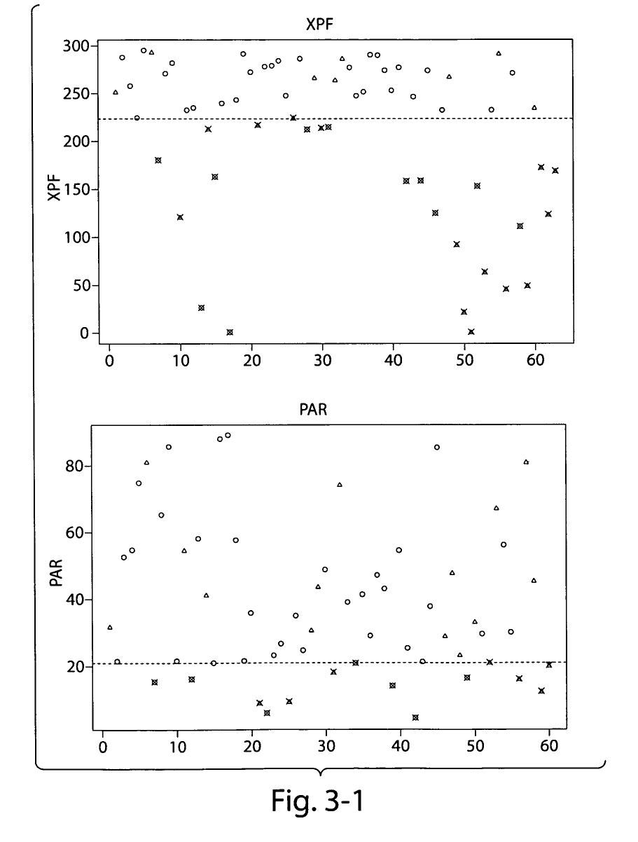

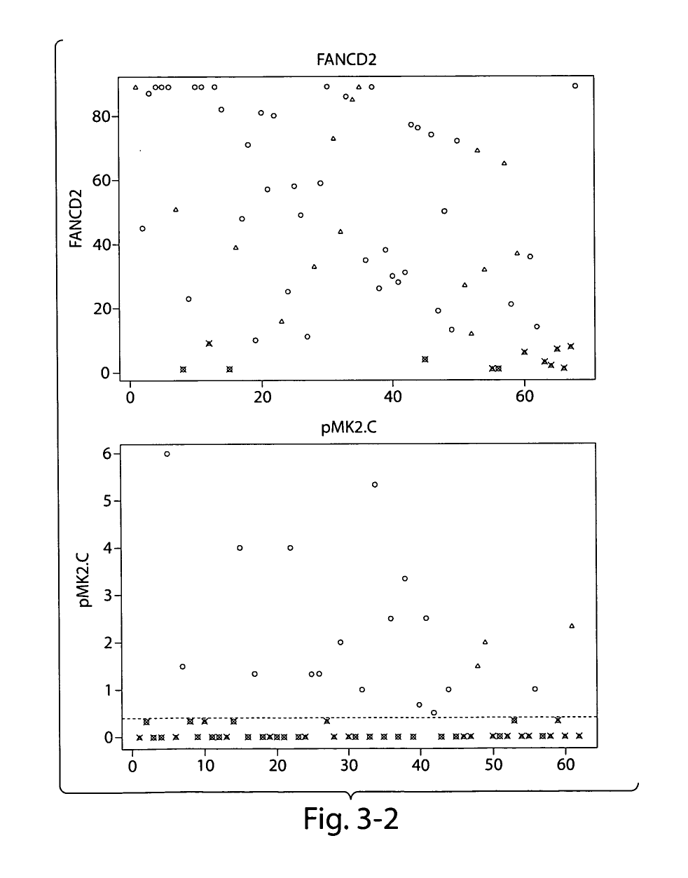

Eleven biomarkers were analyzed for their ability to predict the likelihood of disease recurrence. Rach TNBCMARKER was then evaluated for the separation between between recurrence and non-recurrence groups (FIG. 3). Univariate Cox proportional hazards models were constructed for each of the markers to examine their potential predictive powers. Low XPF (p=0.005), pMK2 (p=0.01), MLH (p=0.007) and FANCD2 (p=0.001) were associated with shorter time to recurrence on univariate analysis (Table 2). For several other markers in DNA repair such as PAR and PARP1, the same analysis failed to reach statistical significance. Ki67, a cell proliferation marker, was significant (p=0.07), as was the p53 tumor suppressor (p=0.02), observations consistent with previous information.

### Example 4

### Discovery of a Multiple DNA Repair Biomarker Panel that Distinguishes Recurrence Groups

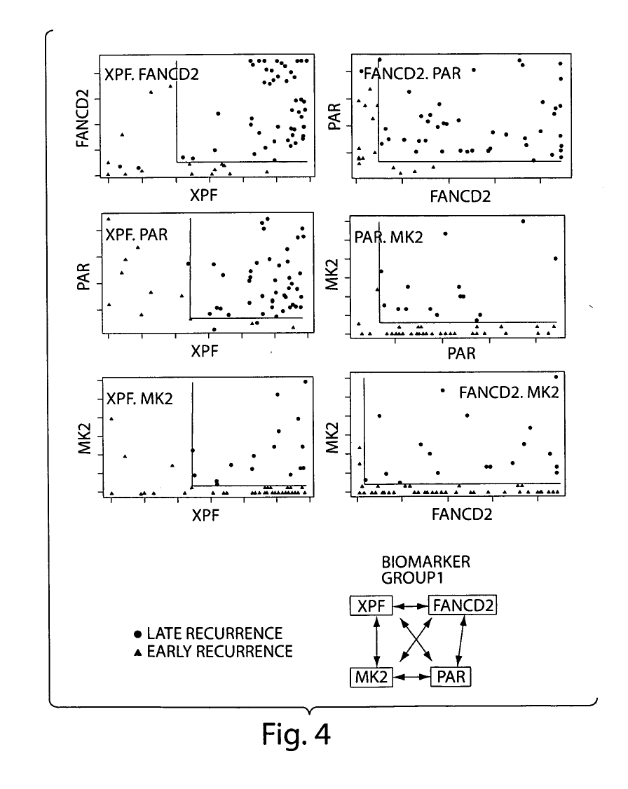

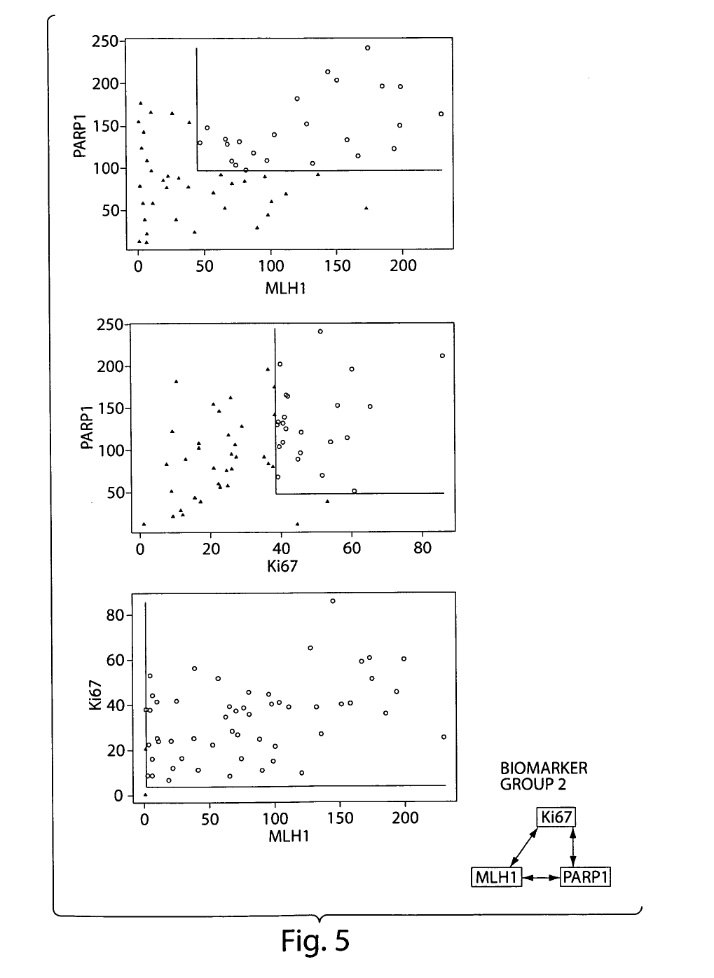

The DNA repair pathways may operate in cell survival and chemotherapy responses in a concerted way. Therefore, DNA repair protein changes may be more effectively determined by combining the effects of markers, rather than by individual analysis. In order to develop a statistically-driven hypothesis for these associations, the combination of two markers were analyzed in stepwise binary marker models using distributive partitioning. Group 1 biomarkers were resolved by a demonstration of stratification benefit when markers were combined in pairs, rather than used individually. The outputting of marker comparisons indicated that XPF, FANCD2, pMK2.C, and PAR based on two-marker analysis. For these four markers in the test, separation of Early versus Late Recurrence groups was better defined from each of the six pairwise marker combinations (FIG. 4). A second group of biomarkers, Group 2, were also resolved by the pairwise analysis (FIG. 5). Other markers did not perform consistently in similar pairwise tests, were not observed to belong to another group, and did not contribute to greater discrimination of the patient recurrence groups. All two marker models were computed for the TNBCMARKERS XPF, pMK2, PAR, PARP1, MLH, FANCD2, ATM, RAD51, BRCA1, ERCC1, NQO1, p53, Ki67 (Table). Statistical evaluation included p-value, Apparent Error Rate, Relative Risk, Odds Ratio, Positive predictive power, and Negative predictive power. Likewise, all three marker models were computed for the TNBCMARKERS XPF, pMK2, PAR, PARP1, MLH, FANCD2, ATM, RAD51, BRCA1, ERCC1, NQO1, p53, Ki67 (Table).

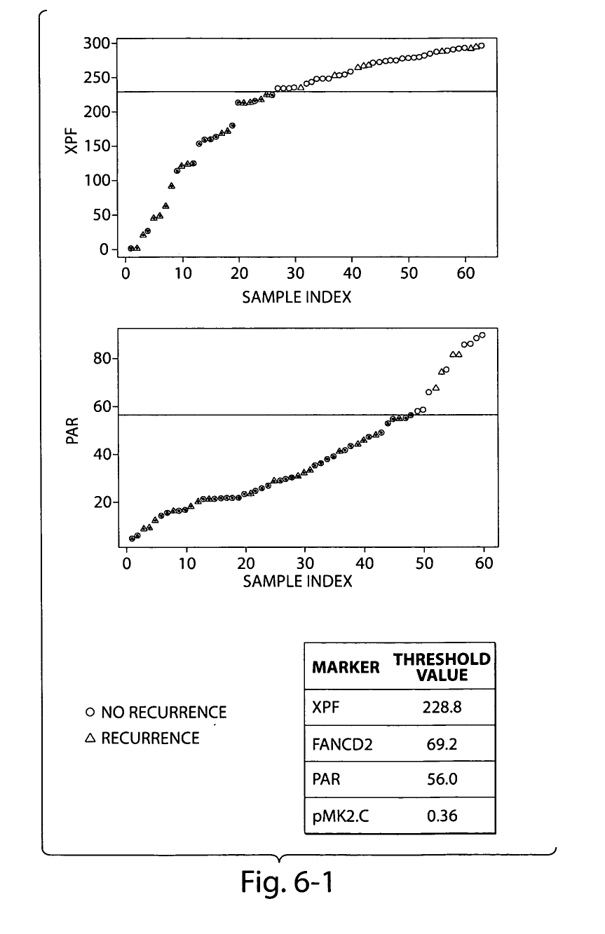

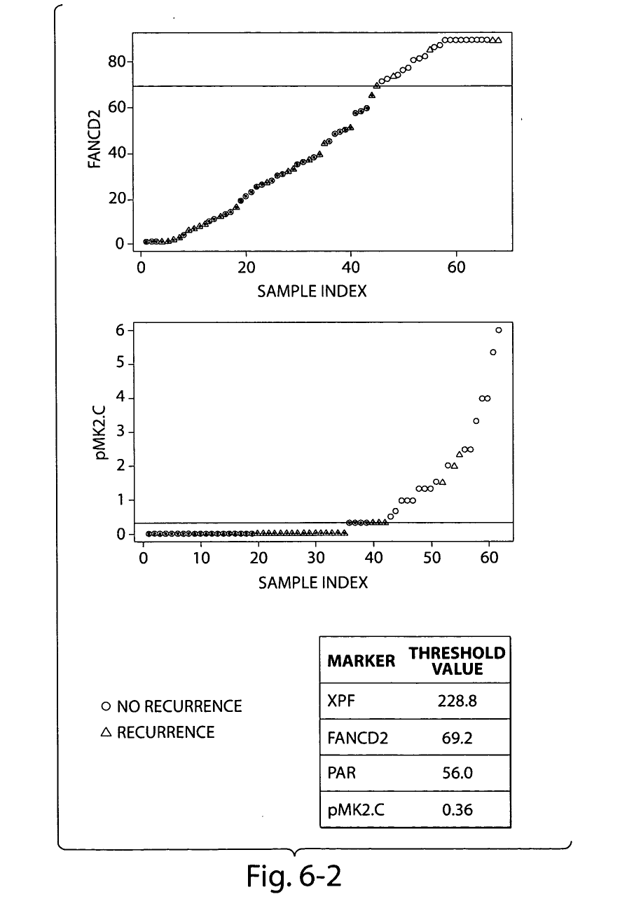

In order to evaluate the combinations of markers in a multi-marker algorithm by partition analysis, the optimal thresholds for separating the samples into likely to recur (Early Recurrence) and not likely to recur (Late Recurrence) groups were first established, and then the time to event curves for the groups compared. The significance of these results were checked by using the computed thresholds to partition the training dataset and comparing the time to event curves of test dataset to the time to event curves for the training dataset. In order to determine thresholds signifying a division in the marker expression levels, the range of each marker was divided into 20 equal intervals and all combinations of thresholds for the four markers in the model are tested. The Thresholds that best separated samples by survival curve p-value are XPF=229, FANCD2=69, PAR=56, pMK2.C=0.36 corresponding to the 0.39, 0.66, 0.71, and 0.62 quantiles of the marker data (FIG. 6).

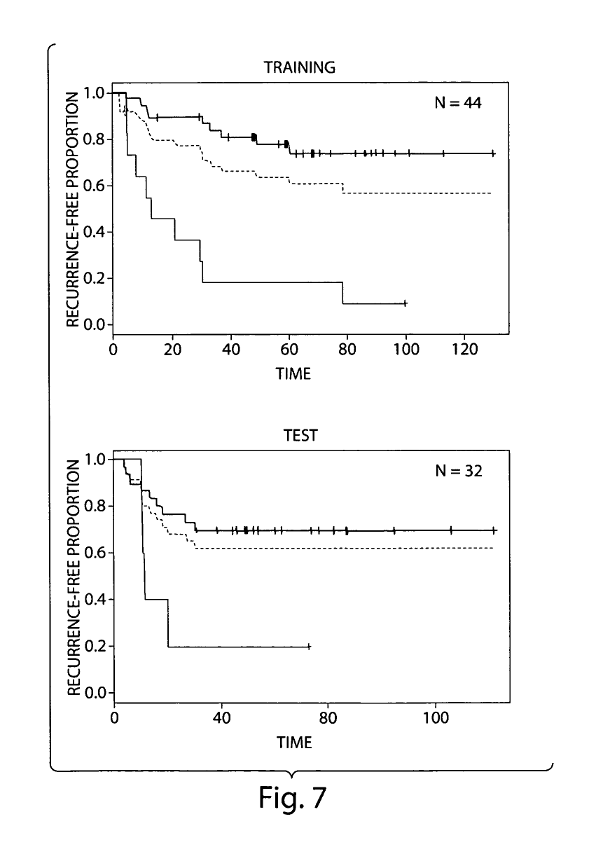

Elevated levels for all four markers were indicative of elevated risk of recurrence with the likely to recur group containing 12 samples (10 recurrences) and the not likely to recur group containing 44 samples (10 recurrences). Strikingly, the likely to recur and not likely to recur groups for Time to Recurrence yields a p-value of 9.05E-07 indicating a significant difference in risk for the two groups as measured in the training dataset (FIG. 7). To independently validate these findings, the Test dataset, which separates the samples into likely to recur (Early Recurrence) group containing 5 samples (4 recurrences) and the not likely to recur (Late Recurrence) group containing 32 samples (9 recurrences), was further interrogated. For the test dataset, the comparison of time to recurrence curves between the likely to recur and not likely to recur groups yielded p-value of 0.0186 that was statistically significant.

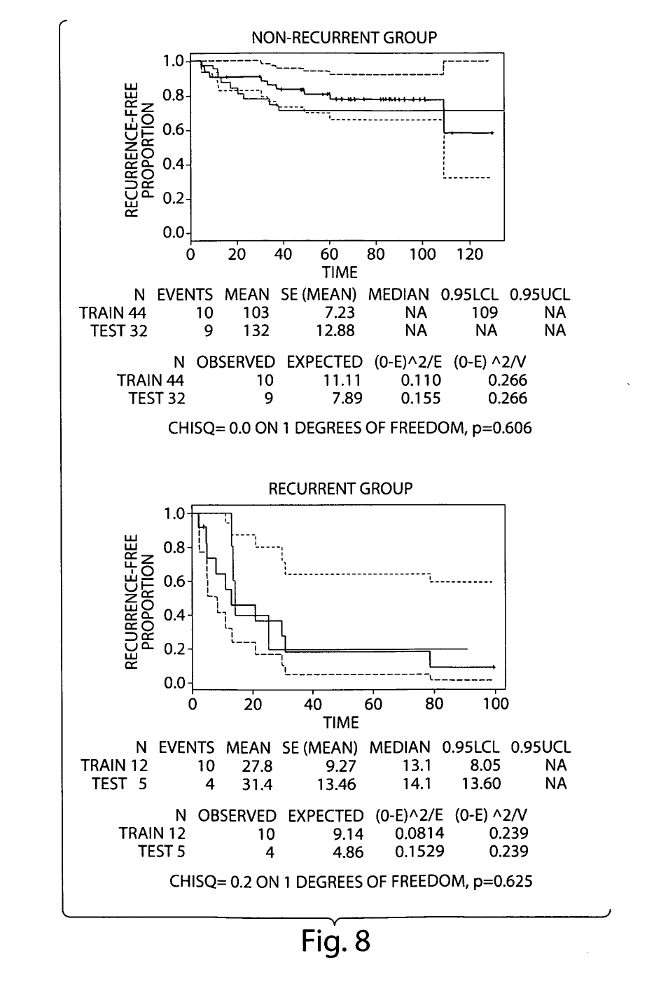

To demonstrate that the two outcome groups from the two datasets were similar, a second cross-validation calculation was conducted. Comparing the time to recurrence curves for the likely to recur group from test dataset and training dataset yielded p-value of 0.625 indicating that the Kaplan-Meier curves were not different between training and test data sets and the likely to recur groups have similar recurrence risk in both datasets (FIG. 8). The comparison of recurrence curves for the not likely to recur group from test dataset and training dataset has a p-value of 0.606 indicating that there was no detectable difference for the likely to recur group between the datasets (FIG. 8).

The low risk group defined by a four DNA repair marker model (PAR, pMK2, XPF, FANCD2) had a mean time to recurrence of 103 months, whereas high risk group had a mean time to recurrence of 28 months [Training cohort]. The model produced similar results (mean time-to-recurrence 134 versus 31 months, p=0.029) in the Test cohort. This was superior to the single markers and to other markers such as P53 (p=0.02) or Ki67 (p=0.07).

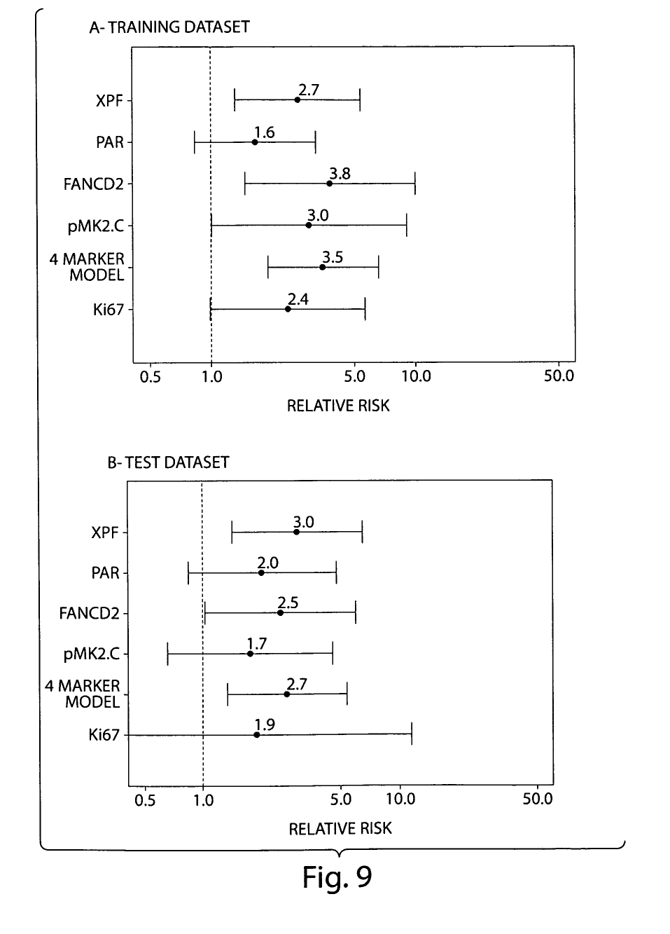

In addition to mean time to recurrence, the low risk (Late Recurrence) and high risk (Early Recurrence) groups were distinct based on Relative Risk (RR). It was found that the four marker model RR=3.52 (1.9-6.6 with 95% CI range) for the Training dataset, and for the Test dataset RR=2.67 (1.3-5.4 with 95% CI range) (FIG. 9). Importantly, Relative Risk calculations for the markers individually, and for non-DNA repair markers such as p53 or Ki67, were not of as high value (2.1 and 1.9 respectively). Likewise, the Apparent Error Rate (AER), an indicator of the level of false positivity to the test, was determined for individual markers and the four marker model. It was found that the four DNA repair marker algorithm yielded a lower AER (0.22), compared to any of the markers individually (0.30-0.52), or other markers such as p53 (0.35) or Ki67 (0.39).

It was further determined that four marker test demonstrated an improvement in identifying patients that were properly grouped based on several specificity/sensitivity criteria. AUC values for the four individual markers were FANCD2 (0.71), pMK2 (0.65), XPF (0.67), and PAR (0.54), compared with a significantly higher AUC value of 0.774 for the four DNA repair marker model determined by a probability analysis for the four marker panel. Positive predictive power and negative predictive power calculations were utilized. Individual markers showed Positive predictive power (0.40-0.57) and Negative predictive power (0.68-0.91). Instead, the four marker algorithm of Xpf, FANCD2, pMK2, and PAR exhibited a Positive predictive power (0.83) and Negative predictive power (0.76) that was superior. As for other statistical metrics, the determinations of positive and negative predictive power proved that a four marker test was more significant and reliable than testing with individual markers.

In addition to the 4 marker model from XPF, FANCD2, PAR, and pMK2, additional alternative 3 marker models and 4 marker models were assessed by a family of the same statistical criteria (Tables). All three marker models with eight TNBCMARKERS (ATM, BRCA1, PAR, MLH1, XPF, FANCD2, PMK2, RAD51) were computed and the lists prioritized by statistical values. The top thirty models were priority ranked for p-value, AER, Relative Risk, Positive Power, and Negative Power. In each case all eight TNBCMARKERS were populated in the top thirty models (Table). Minimum and maximum ranges for the top thirty models were sorted for p-value (2.94e-05-1.02e-03, AER (0.22-0.27), Relative Risk (2.88-4.02), Positive Power (0.59-0.64), Negative Power (0.72-0.78), and shown to be superior to single TNBCMARKERS. These data show that there are multiple three and four marker models with the TNBCMARKERS that show significant improvements over single TNBCMARKER and other marker tests.

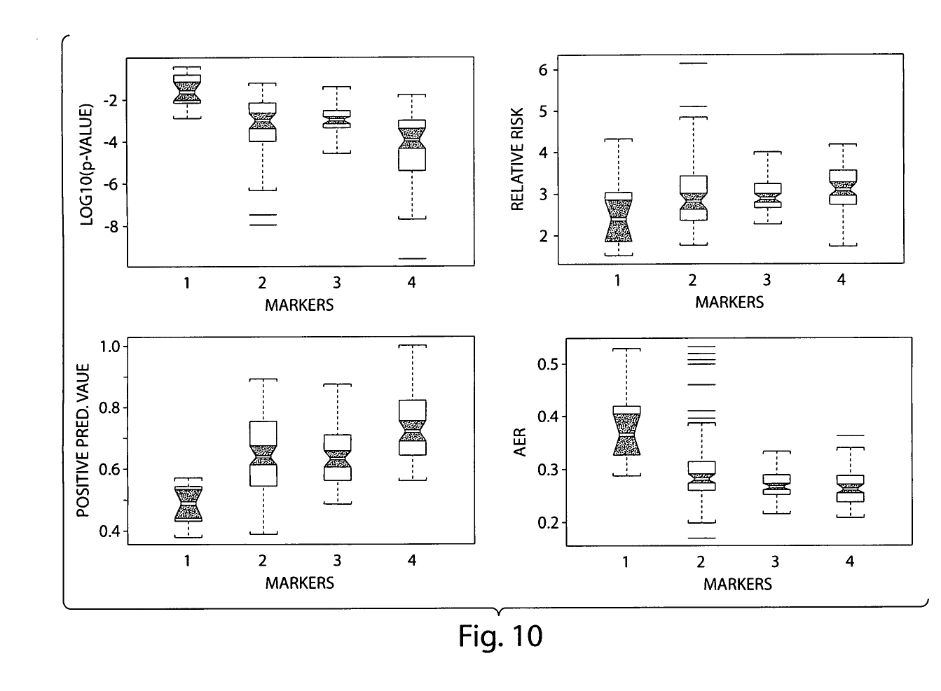

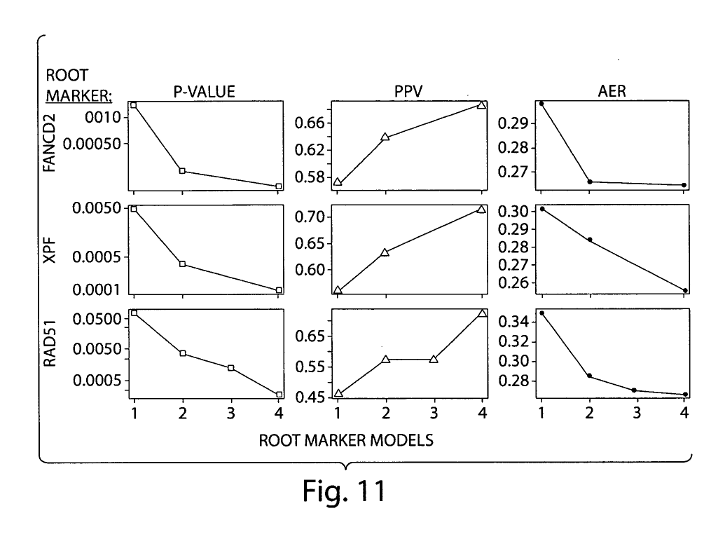

To demonstrate that TNBCMARKERS show improved performance over single markers, the partition analysis output was evaluated against the six statistical values from the output and a comparison of the 1-, 2-, 3-, and 4-marker models with the group of DNA Repair markers (XPF, pMK2, PAR, PARP1, MLH, FANCD2, ATM, RAD51, BRCA1, ERCC1, NQO1,). The results indicate that based on the values of P-value, Relative Risk, Positive Predictive Value, Specificity, and AER (FIG. 10), that increasing the number of markers from this group in the model leads to an increased performance where 3-, 4-, and 5-marker models are clearly superior and non-overlapping with the 1-marker models. Therefore, the four TNBCMARKER tests and the five TNBC MARKER tests give better discrimination and fewer errors than a single DNA repair marker. An alternative demonstration of the importance of the multimarker models is shown by considering one of the TNBCMARKERS as a root marker for all models. The statistical values of log 10P-value, Positive Predictive Value (PPV), and AER were computed for a 1-marker model with either the FANCD2, XPF, or RAD51 TNBCMARKERS. Next the same statistical tests were generated with all the models containing FANCD2, XPF, or RAD51 and the median value for all the 2-, 3- or 4-marker models calculated. In each of the three cases, the 2-, 3- and 4-marker models show a trend to increased performance with addition of markers that is significantly improved over the FANCD2, XPF, or RAD51′-marker models (FIG. 11). Like the calculations with all TNBCMARKERS, increased performance features are associated with co-evaluation of markers in 2-, 3-, and 4-marker models.

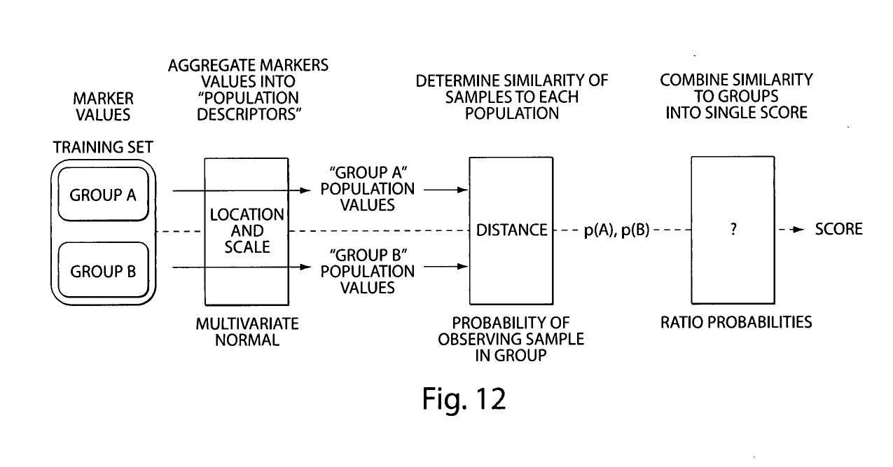

A probability analysis statistical process was independently executed to compare the TNBCMARKERS XPF, pMK2, PAR, PARP1, MLH, FANCD2, ATM, RAD51, BRCA1, ERCC1, NQO1, p53, Ki67. A procedure was developed to examine the placement of a patient in an Early Recurrence or Late Recurrence group by examining the probability of observing the marker evaluation in each group (FIG. 12). In this procedure, we refine the definition of group membership used in the above analysis by defining a region of low incidence of recurrence in addition to the region of high incidence of recurrence. These regions are constructed using multivariate probability distributions for the likely to recur and not likely to recur groups and a single score reflecting group membership is constructed from the individual group probabilities. One method of constructing these probability distributions is to use a parametric estimation of the probabilities, i.e. normal distributions. Another method is to use a non-parametric (distribution free) estimate of the probability densities for each group.

Parametric Method (Normal Distribution):

By measuring a mean vector, μ, and covariance matrix, Σ, for both groups, the probability density function can be evaluated for the not likely to recur, fₙₗ(x), and the likely to recur, f₁(x), groups given the marker values, x.

$$\text{?} = ( \frac{\text{?}}{\text{?}} ) exp ( - \frac{1}{2} ( \text{?} - \text{?} ) \text{?} ( \text{?} ) ]$$

$$\text{?} \text{indicates text missing or illegible when filed}$$

The probability densities are expressed as a posterior probability of observing the marker values in each group.

$$\text{?} = \frac{\text{?}}{\text{?}} and P ( l \vert x ) = \frac{\text{?}}{\text{?}}$$

$$\text{?} \text{indicates text missing or illegible when filed}$$

In order to obtain a scalar value to simplify interpretation these probabilities are combined into a score, s, via

$$s ( x ) = \frac{P ( nl ) - P ( n )}{P ( nl ) + P ( l )}$$

This form for the score is chosen so that a sample with much higher probability of being observed in the not likely to recur group (P(nl)>>P(1)) has a score close to +1; when the probability of being observed in the likely to recur group is much higher the score is close to −1. If the sample has nearly equal probability of being observed in both groups the score is close to zero. In order to accommodate samples where the outcome is unclear from the model, the magnitude of the score must exceed a threshold of ±⅓ before assigning to a group. A score of ±⅓ is equivalent to a 2-fold difference in group membership probability: P(nl)=2*P(1) or 2*P(nl)=P(1). If a sample does not exceed the threshold values, it is assigned to neither group and classed as indeterminate.

The mean and covariance matrices for each group are calculated from the dataset and are used to generate scores for a validation set.

Models using all unique combinations of one, two, three, and four markers were constructed and checked for their ability to discriminate patient's outcome. The number of samples that was indeterminate is plotted for all models. The median number of samples that fall in the indeterminate range (−⅓<score<⅓) decreases as more markers are added to the model. Outputs were evaluated in four ways: 1) Scores by Outcomes, 2) Kaplan-Meier Recurrence Curve, 3) Predicted Outcome from Score, and 4) ROC Plot from Score. Scores are probabilities of Recurrence or No Recurrence and thus range from −1 to 1. Also, the Likelihood of an Event is also set to range between 0 and 1.

Scores by Outcomes indicates the likelihood of recurrence for a patient given their score. Likelihood of recurrence is plotted on the y-axis. A patient's recurrence likelihood is determined by reading the y-value from the curve corresponding to the x-value (score). The indeterminant region, as defined above, is reflected in the plotting strategy as indicated by dashed lines and is (−⅓<score<⅓).

Predicted Outcome from Score is an assessment of the clinical relevance of the score by computing the likelihood of recurrence given a score value. The probability of recurrence for each level of score is calculated by binning all the patients within a score window (i.e. −1≦score≦0.8) and determining the percentage of patient samples within the window experiencing recurrence. Bins where the number of samples is less than 2 are not reported. The trend of the probability of recurrence vs. score is approximated using a Loess fit and the point-wise 95% confidence interval for the trend line is also reported (dotted lines in figures).

In addition, the ROC Plot from Score was used a determination of the quality of the test. The choice of ±⅓ for the indeterminate score threshold may not be optimal. The effect of choosing different score thresholds in assigning group membership can be examined using a ROC plot. A ROC plot is constructed from the score by moving a threshold from −1 to 1 and calling all samples less than the threshold positive for recurrence or likely to recur. All samples with scores greater than the threshold are allocated to the not likely to recur group. The percentage of all recurrent samples correctly detected is plotted against the percentage of non-recurrent samples incorrectly identified as recurrent.

Single TNBCMARKER Probability Analysis

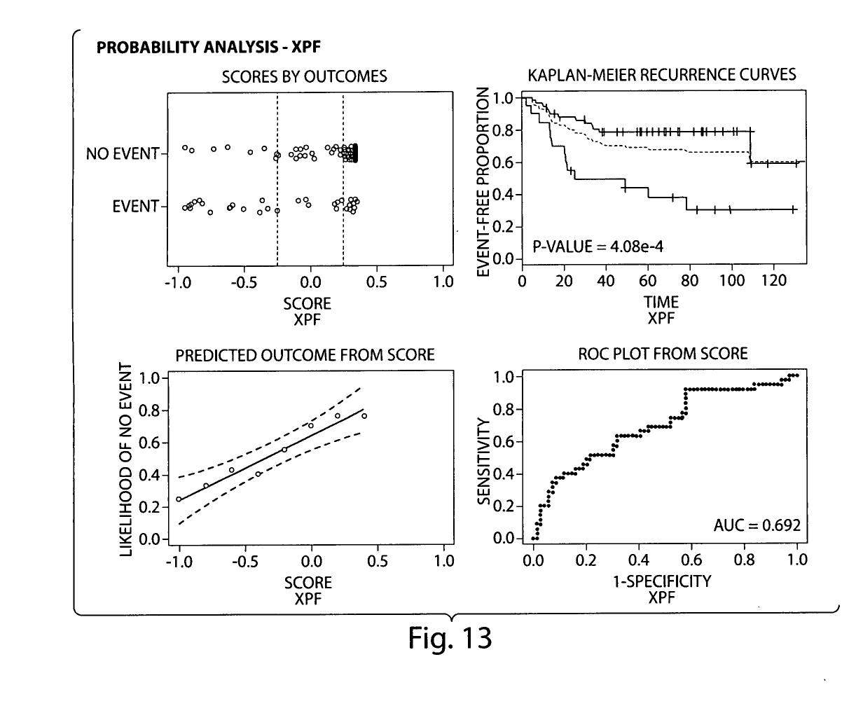

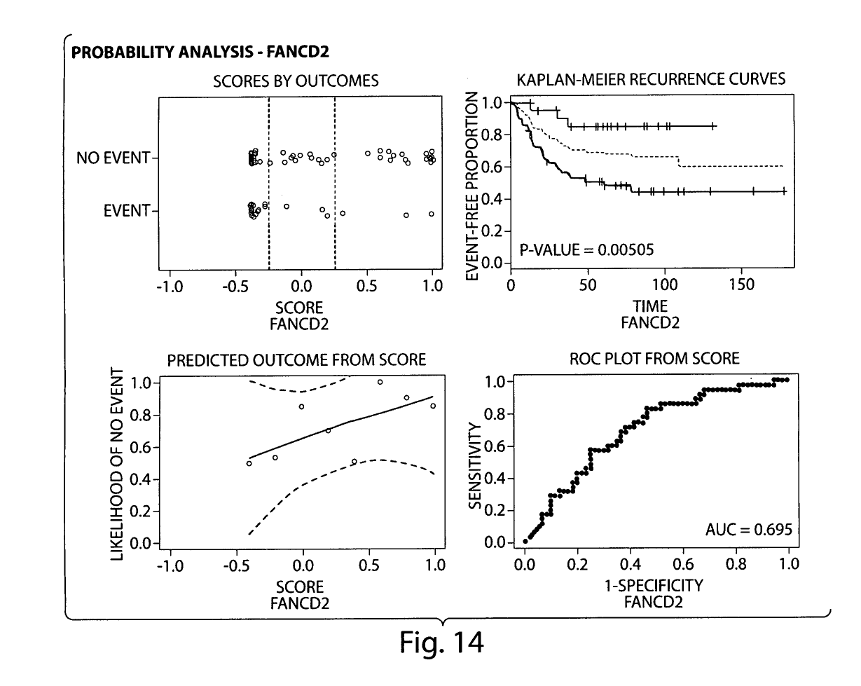

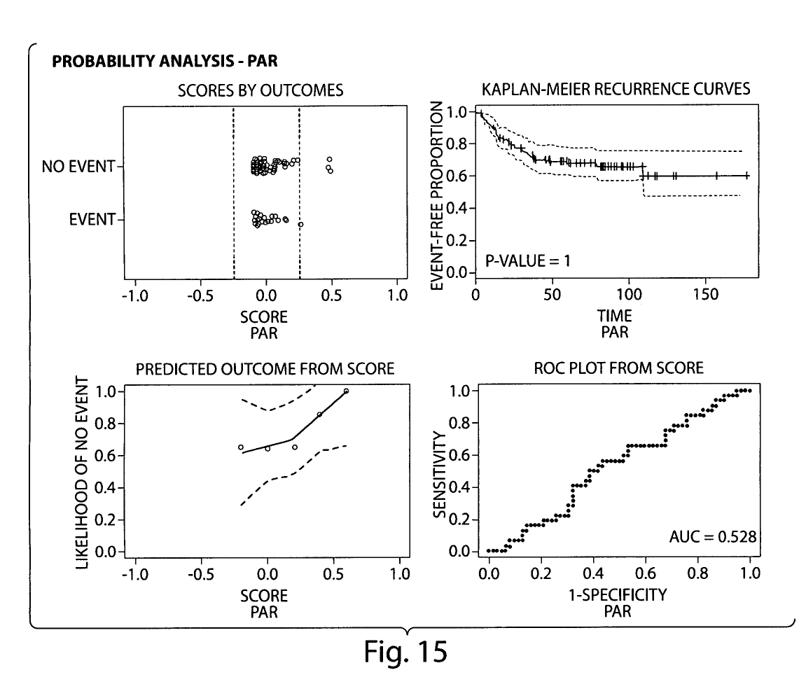

The Scores by Outcomes for all patient samples are separated by clinical outcome and are plotted for single TNBC markers XPF, FANCD2, and PAR (FIGS. 13-15), Scores by Outcome, top left). Likewise, Kaplan-Meier Recurrence curves were plotted for XPF, FANCD2, and PAR (FIGS. 13-15), Kaplan-Meier Recurrence Curve). In addition, the Predicted Outcome from Score was plotted for XPF, FANCD2, and PAR (FIGS. 13-15, Predicted Outcome from Score; bottom left). Table indicates the relative values for Probability Analysis for single TNBC Marker tests.

The second analysis with single TNBC markers was the computing of Kaplan-Meier Recurrence curves, illustrated with the markesr XPF, FANCD2, and PAR (FIG. 13-15, Kaplan_Meier Recurrence Curves, top right). The Early Recurrence and Late Recurrence subgroups are designated in the figures and a p-value indicating the separation of the groups is shown.

The single TNBC markers were also evaluated for a ROC Plot from Score criteria (FIGS. 13-15; ROC Plot from Score, bottom right). AUC values are listed for XPF (0.692), FANCD2 (0.695), and PAR (0.526) on the Figures.

Multiple TNBCMARKER Probability Analysis

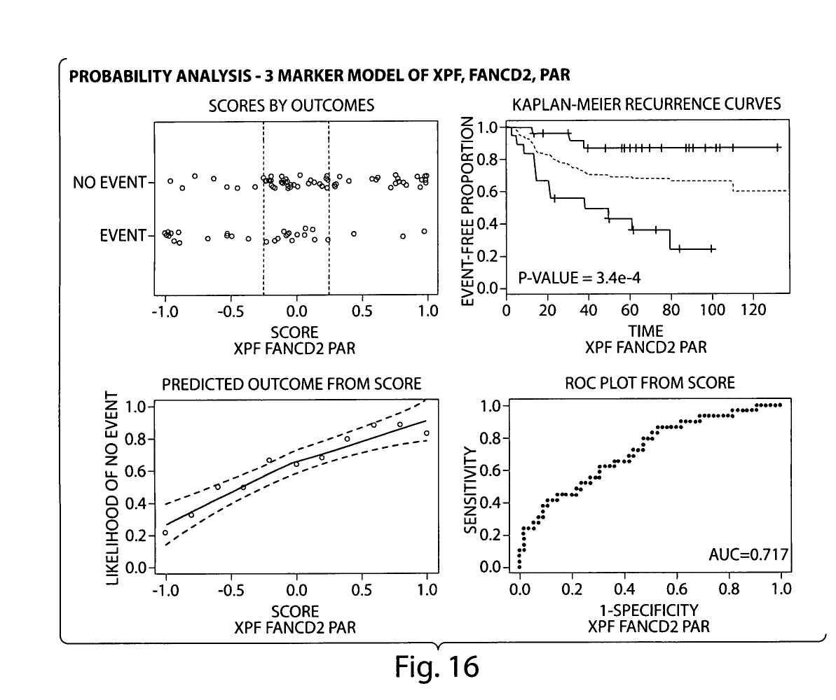

TNBCMARKER Probability Analysis was also constructed in two- and three-marker models from the TNBC markers (Table). For the XPF, FANCD2, PAR three marker model there was an increased significance for the Scores by Outcomes, Kaplan-Meier Recurrence curve (p=3.4e-4), Predicted Outcome from Score, and ROC plot (AUC=0.717) indicative of better discrimination and fewer errors in a three TNBC marker test over any of the TNBC single marker tests (FIG. 16).

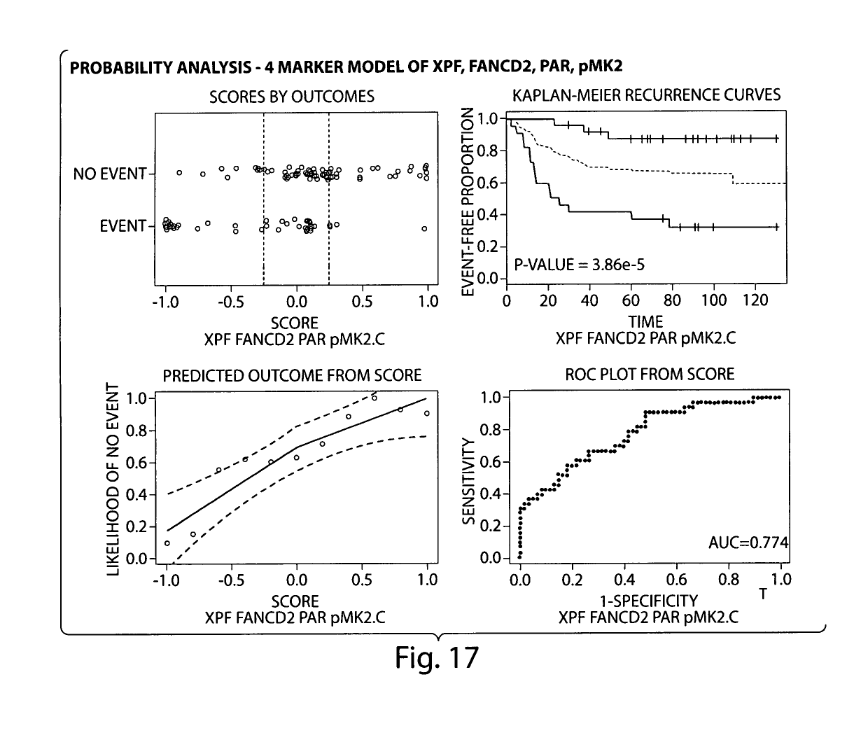

TNBCMARKER Probability Analysis was also constructed in several four-marker models from the TNBC markers (Table). For the XPF, FANCD2, PAR, PMK2 four marker model there was an increased significance for the Scores by Outcomes, Kaplan-Meier Recurrence curve (p=3.86e-5), Predicted Outcome from Score, and ROC plot (AUC=0.774) indicative of an improvement in the results of the test over the TNBC single marker tests (FIG. 17). From the Predicted Outcome from Score it can be seen that of the samples with scores less than −0.9, approximately 20% had a recurrence and of the samples with a score greater than 0.9 approximately 90% had a recurrence. Samples with scores close to zero had close to a 50% chance of recurrence. With ROC analysis, 40% of the recurrent samples are detected before 10% of the non-recurrent samples are incorrectly identified using a score threshold of −0.54. Slightly more than 50% of the non-recurrent samples are detected before 10% of the recurrent samples are incorrectly identified as non-recurrent. Therefore, the four TNBC marker test gives better discrimination and fewer errors than a single DNA repair marker.

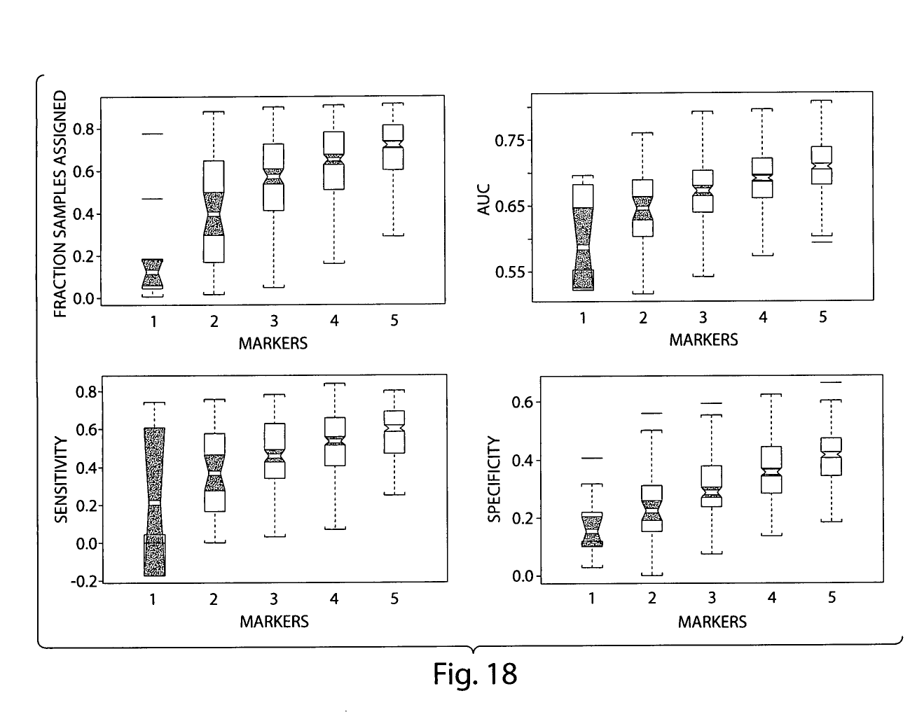

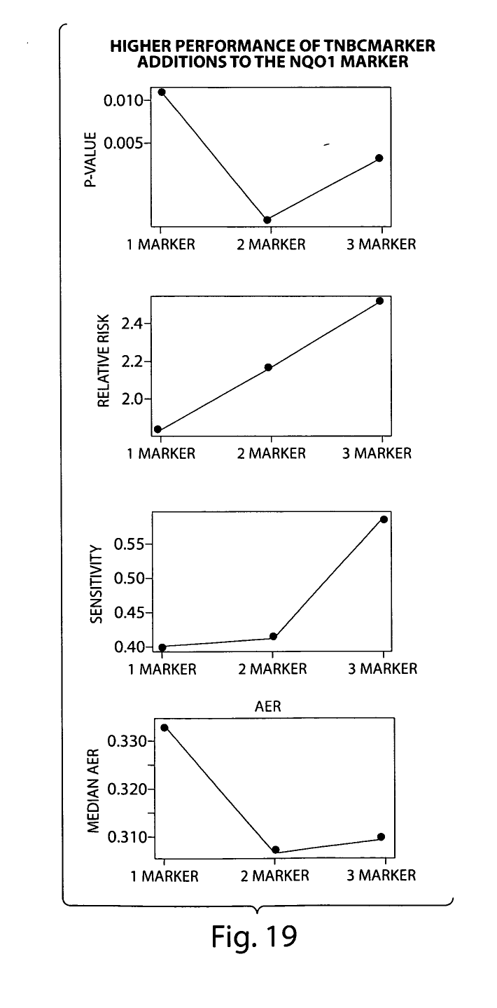

To demonstrate that TNBCMARKERS show improved performance over single markers, the probability analysis output was evaluated against the four statistical values from the output and a comparison of the 1-, 2-, 3-, 4-, and 5-marker models with the group of DNA Repair markers (XPF, pMK2, PAR, PARP1, MLH, FANCD2, ATM, RAD51, BRCA1, ERCC1, NQO1,). The results indicate that based on the values of Fraction Sample Assigned, AUC, Sensitivity, and Specificity (FIG. 18), that increasing the number of markers from this group in the model leads to an increased performance where 3-, 4-, and 5-marker models are clearly superior and non-overlapping with the 1-marker models. Therefore, the four TNBCMARKER tests and the five TNBC MARKER tests give better discrimination and fewer errors than a single DNA repair marker

Additional markers such as NQO1 that are not commonly recognized in DNA repair pathways may yield significant associations when used in similar multimarker algorithms as above. In single marker testing of Early versus Late Recurrence it was observed that the marker showed log 10p-value (p=1.14E-02), PPV (0.50), and AER (0.33). To demonstrate the ability of the NQO1 marker to associate with DNA repair to better inform outcomes in breast cancer, NQO1 was tested with TNBCMARKERS in 2-, and 3-marker models. It is shown that the median 2- and 3-marker model values for p-value, PPV, and AER are a general improvement on the performance of NQO1 by itself.

TABLE 1
Biomarkers in the invention

| MARKER | CLASS | PATHWAY |
|---|---|---|
| FANCD2 | DNA REPAIR | FA/HR |
| XPF | DNA REPAIR | Nucleotide Excision |
| | | Repair |
| PAR | DNA REPAIR | Base Excision Repair |
| PhosphoMapKapKinase2 | DNA Damage | FA/HR |
| (pMK2) | Signaling | |
| MLH1 | DNA REPAIR | Mismatich Repair |
| PARP1 | DNA REPAIR | Base Excision Repair |
| ATM | DNA REPAIR | FA/HR and NHEJ |
| RAD51 | DNA REPAIR | FA/HR |
| BRCA1 | DNA REPAIR | FA/HR |
| ERCC1 | DNA REPAIR | Nucleotide Excision |
| | | Repair |
| P53 | Tumor Suppressor | |
| Ki67 | Proliferation | |
| NQO1 | Detoxificiation | |
| Cytokeratin | Epithelial | |
| Vimentin | Surface Marker | |
| TRP | Protein | |
| PSTAT | Protein Phospho | |

TABLE 2
Univariate and partition analysis biomarker output data from
Training cohort

| | | | | | Positive | Negative | |
|---|---|---|---|---|---|---|---|
| Biomarker | p-value^{a} | AER^{b} | Relative Risk | Odds Ratio | Power | Power | AUC^{c} |
| FANCD2 | 1.41E−03 | 0.30 | 3.83 | 7.52 | 0.57 | 0.85 | 0.71 |
| XPF | 4.97E−03 | 0.30 | 2.66 | 4.77 | 0.56 | 0.79 | 0.67 |
| PAR | 2.93E−01 | 0.35 | 1.64 | 2.29 | 0.50 | 0.70 | 0.54 |
| pMK2 | 1.16E−02 | 0.42 | 3.02 | 4.68 | 0.45 | 0.85 | 0.65 |
| PARP1 | 2.59E−01 | 0.41 | 1.50 | 1.88 | 0.43 | 0.71 | 0.53 |
| MLH1 | 1.72E−02 | 0.37 | 2.34 | 3.61 | 0.48 | 0.79 | 0.61 |
| P53 | 2.42E−02 | 0.35 | 2.06 | 3.11 | 0.50 | 0.76 | 0.60 |
| Ki67^{&} | 7.03E−02 | 0.39 | 2.36 | 3.50 | 0.45 | 0.81 | 0.59 |
| 4-marker^{#} | 9.05E−07 | 0.22 | 3.52 | 16.11 | 0.83 | 0.76 | na |

^{a}p-value for separation of Early Recurrence from Late Recurrence groups
^{b}AER, Apparent Error Rate
^{c}AUC, Area Under Curve value from Receiver Operator Characteristics
^{&}Ki67 quantity score, weighting used is 0111 for 0, 1+, 2+, 3+ bins
^{#}4-marker, multi-marker model containing FANCD2, XPF, PAR, pMK2

TABLE 3
Summary of Top Thirty Partition Analysis
Three marker models for TNBCMARKERS

| *Markers Represented | Value | Manimum | Maximum |
|---|---|---|---|
| 8/8 | p-Value | 2.94e−5 | 1.02e−3 |
| 8/8 | AER | 0.22 | 0.27 |
| 8/8 | Relative Risk | 2.88 | 4.02 |
| 8/8 | Positive Power | 0.59 | 0.64 |
| 8/8 | Negative | 0.72 | 0.78 |
| | Power | | |

*Markers in three marker model analysis were ATM, BRCA1, PAR, MLH1, XPF, FANCD2, PMK, and RAD51

TABLE 4
One Marker Partition Analysis TNBCMARKERS

| Markers | Marker | pval | AUC | Sens | Spec | PosPow | NegPow | AER | RelRisk |
|---|---|---|---|---|---|---|---|---|---|
| FANCD2 | FANCD2 | 1.41E−03 | 0.71 | 0.81 | 0.64 | 0.57 | 0.85 | 0.30 | 3.83 |
| BRCA1 | BRCA1 | 3.95E−03 | 0.59 | 0.57 | 0.78 | 0.57 | 0.78 | 0.29 | 2.60 |
| XPF | XPF | 4.97E−03 | 0.67 | 0.64 | 0.73 | 0.56 | 0.79 | 0.30 | 2.66 |
| NQO1 | NQO1 | 1.14E−02 | 0.61 | 0.40 | 0.80 | 0.50 | 0.73 | 0.33 | 1.83 |
| PMK2 | PMK2 | 1.16E−02 | 0.65 | 0.86 | 0.43 | 0.45 | 0.85 | 0.42 | 3.02 |
| MLH1 | MLH1 | 1.72E−02 | 0.61 | 0.73 | 0.58 | 0.48 | 0.79 | 0.37 | 2.34 |
| P53 | P53 | 2.42E−02 | 0.60 | 0.59 | 0.68 | 0.50 | 0.76 | 0.35 | 2.06 |
| Ki67 | Ki67 | 7.03E−02 | 0.58 | 0.75 | 0.54 | 0.45 | 0.81 | 0.39 | 2.36 |
| ATM | ATM | 7.28E−02 | 0.51 | 0.95 | 0.21 | 0.40 | 0.88 | 0.53 | 3.20 |
| ERCC1 | ERCC1 | 1.20E−01 | 0.56 | 0.95 | 0.24 | 0.39 | 0.91 | 0.52 | 4.31 |
| RAD51 | RAD51 | 1.42E−01 | 0.57 | 0.55 | 0.70 | 0.46 | 0.77 | 0.35 | 1.99 |
| Ki67 | Ki67 | 1.58E−01 | 0.53 | 0.87 | 0.27 | 0.38 | 0.80 | 0.53 | 1.89 |
| PARP1 | PARP1 | 2.59E−01 | 0.53 | 0.55 | 0.61 | 0.43 | 0.71 | 0.41 | 1.50 |
| PAR | PAR | 3.98E−01 | 0.54 | 0.35 | 0.81 | 0.54 | 0.67 | 0.37 | 1.62 |

TABLE 5
Two Marker Partition Analysis TNBCMARKERS

| Markers | pval | Sens | Spec | PosPow | NegPow | AER | RelRisk |
|---|---|---|---|---|---|---|---|
| ERCC1, MLH1 | 1.23E−08 | 0.25 | 0.97 | 0.83 | 0.71 | 0.28 | 2.89 |
| ERCC1, BRCA1 | 3.19E−08 | 0.38 | 0.98 | 0.89 | 0.75 | 0.23 | 3.56 |
| XPF, PMK2 | 3.60E−08 | 0.52 | 0.95 | 0.85 | 0.79 | 0.20 | 3.98 |
| BRCA1, PMK2 | 5.57E−07 | 0.55 | 0.89 | 0.73 | 0.79 | 0.23 | 3.42 |
| FANCD2, ATM | 8.58E−07 | 0.71 | 0.90 | 0.80 | 0.84 | 0.17 | 5.12 |
| ERCC1, FANCD2 | 2.02E−06 | 0.42 | 0.94 | 0.80 | 0.74 | 0.25 | 3.13 |
| BRCA1, ATM | 2.10E−06 | 0.37 | 0.97 | 0.88 | 0.72 | 0.25 | 3.14 |
| FANCD2, P53 | 2.13E−06 | 0.50 | 0.94 | 0.83 | 0.76 | 0.22 | 3.50 |
| XPF, P53 | 3.89E−06 | 0.41 | 0.95 | 0.82 | 0.74 | 0.25 | 3.15 |
| NQ01, BRCA1 | 7.98E−06 | 0.30 | 0.95 | 0.75 | 0.73 | 0.27 | 2.79 |
| ERCC1, XPF | 1.25E−05 | 0.29 | 0.95 | 0.75 | 0.71 | 0.28 | 2.60 |
| MLH1, ATM | 1.54E−05 | 0.68 | 0.79 | 0.65 | 0.81 | 0.25 | 3.47 |
| Ki67, XPF | 1.99E−05 | 0.27 | 0.98 | 0.86 | 0.71 | 0.27 | 3.00 |
| NQ01, PMK2 | 2.50E−05 | 0.42 | 0.89 | 0.67 | 0.75 | 0.27 | 2.67 |
| FANCD2, PMK2 | 3.13E−05 | 0.33 | 0.97 | 0.88 | 0.70 | 0.27 | 2.94 |
| XPF, ATM | 3.45E−05 | 0.53 | 0.85 | 0.67 | 0.76 | 0.26 | 2.81 |
| Ki67, FANCD2 | 3.58E−05 | 0.67 | 0.81 | 0.67 | 0.81 | 0.25 | 3.43 |
| FANCD2, PARP1 | 4.30E−05 | 0.75 | 0.74 | 0.63 | 0.83 | 0.26 | 3.75 |
| BRCA1, P53 | 6.00E−05 | 0.38 | 0.98 | 0.89 | 0.75 | 0.23 | 3.56 |
| Ki67, NQ01 | 9.81E−05 | 0.35 | 0.93 | 0.70 | 0.74 | 0.27 | 2.69 |
| MLH1, PMK2 | 1.11E−04 | 0.57 | 0.82 | 0.63 | 0.78 | 0.27 | 2.81 |
| ERCC1, NQ01 | 1.17E−04 | 0.25 | 0.97 | 0.83 | 0.72 | 0.27 | 2.94 |
| BRCA1, FANCD2 | 1.22E−04 | 0.42 | 0.94 | 0.80 | 0.74 | 0.25 | 3.13 |
| BRCA1, PAR | 1.49E−04 | 0.55 | 0.77 | 0.61 | 0.73 | 0.31 | 2.24 |
| XPF, PAR | 1.97E−04 | 0.30 | 0.97 | 0.86 | 0.68 | 0.29 | 2.69 |
| RAD51, PMK2 | 2.04E−04 | 0.50 | 0.87 | 0.67 | 0.77 | 0.25 | 2.93 |
| PARP1, P53 | 2.41E−04 | 0.32 | 0.98 | 0.88 | 0.72 | 0.26 | 3.15 |
| XPF, PARP1 | 3.29E−04 | 0.59 | 0.79 | 0.62 | 0.78 | 0.28 | 2.75 |
| XPF, FANCD2 | 3.50E−04 | 0.75 | 0.78 | 0.65 | 0.85 | 0.23 | 4.30 |
| NQ01, ATM | 3.74E−04 | 0.42 | 0.84 | 0.62 | 0.71 | 0.31 | 2.13 |
| Ki67, BRCA1 | 4.89E−04 | 0.48 | 0.85 | 0.63 | 0.76 | 0.27 | 2.61 |
| PAR, FANCD2 | 4.97E−04 | 0.78 | 0.66 | 0.58 | 0.83 | 0.30 | 3.35 |
| RAD51, ATM | 5.39E−04 | 0.56 | 0.85 | 0.67 | 0.78 | 0.25 | 3.08 |
| PMK2, ATM | 7.04E−04 | 0.44 | 0.88 | 0.67 | 0.74 | 0.28 | 2.53 |
| BRCA1, XPF | 7.95E−04 | 0.57 | 0.79 | 0.60 | 0.78 | 0.28 | 2.67 |
| NQ01, P53 | 8.74E−04 | 0.30 | 0.92 | 0.67 | 0.72 | 0.29 | 2.38 |
| NQ01, PAR | 9.25E−04 | 0.35 | 0.87 | 0.64 | 0.68 | 0.33 | 1.96 |
| Ki67, P53 | 1.00E−03 | 0.27 | 0.98 | 0.86 | 0.71 | 0.27 | 3.00 |
| RAD51, BRCA1 | 1.03E−03 | 0.47 | 0.93 | 0.75 | 0.79 | 0.22 | 3.60 |
| RAD51, FANCD2 | 1.05E−03 | 0.79 | 0.67 | 0.56 | 0.86 | 0.29 | 3.89 |
| PMK2, P53 | 1.06E−03 | 0.43 | 0.92 | 0.75 | 0.74 | 0.26 | 2.88 |
| NQ01, PARP1 | 1.20E−03 | 0.40 | 0.85 | 0.57 | 0.73 | 0.31 | 2.14 |
| BRCA1, PARP1 | 1.62E−03 | 0.48 | 0.88 | 0.67 | 0.76 | 0.26 | 2.79 |
| PAR, PMK2 | 1.62E−03 | 0.89 | 0.48 | 0.53 | 0.88 | 0.35 | 4.25 |
| MLH1, FANCD2 | 1.73E−03 | 0.80 | 0.64 | 0.55 | 0.85 | 0.30 | 3.72 |
| MLH1, P53 | 1.77E−03 | 0.38 | 0.87 | 0.62 | 0.72 | 0.31 | 2.18 |
| NQ01, XPF | 1.86E−03 | 0.40 | 0.85 | 0.57 | 0.73 | 0.31 | 2.14 |
| PAR, P53 | 1.90E−03 | 0.55 | 0.77 | 0.61 | 0.73 | 0.31 | 2.24 |
| NQ01, FANCD2 | 2.10E−03 | 0.78 | 0.65 | 0.54 | 0.85 | 0.31 | 3.50 |
| MLH1, PAR | 2.46E−03 | 0.60 | 0.68 | 0.55 | 0.72 | 0.35 | 1.98 |
| MLH1, PARP1 | 2.56E−03 | 0.19 | 0.95 | 0.67 | 0.68 | 0.32 | 2.08 |
| ERCC1, P53 | 2.61E−03 | 0.29 | 0.95 | 0.75 | 0.72 | 0.28 | 2.65 |
| P53, ATM | 2.86E−03 | 0.58 | 0.75 | 0.58 | 0.75 | 0.31 | 2.32 |
| Ki67, MLH1 | 3.52E−03 | 0.64 | 0.70 | 0.54 | 0.78 | 0.32 | 2.42 |
| Ki67, PMK2 | 3.76E−03 | 0.86 | 0.48 | 0.48 | 0.86 | 0.39 | 3.48 |
| ERCC1, ATM | 4.34E−03 | 0.95 | 0.38 | 0.47 | 0.92 | 0.41 | 6.16 |
| PMK2, PARP1 | 4.47E−03 | 0.86 | 0.46 | 0.47 | 0.85 | 0.40 | 3.16 |
| ERCC1, PMK2 | 4.66E−03 | 0.90 | 0.46 | 0.47 | 0.89 | 0.39 | 4.50 |
| BRCA1, MLH1 | 5.99E−03 | 0.55 | 0.76 | 0.55 | 0.76 | 0.31 | 2.32 |
| RAD51, XPF | 6.48E−03 | 0.65 | 0.73 | 0.54 | 0.81 | 0.30 | 2.86 |
| XPF, MLH1 | 6.48E−03 | 0.62 | 0.73 | 0.54 | 0.78 | 0.31 | 2.51 |
| NQ01, RAD51 | 6.99E−03 | 0.42 | 0.83 | 0.53 | 0.75 | 0.31 | 2.13 |
| RAD51, P53 | 8.99E−03 | 0.35 | 0.90 | 0.64 | 0.74 | 0.28 | 2.45 |
| NQ01, MLH1 | 9.29E−03 | 0.20 | 0.95 | 0.67 | 0.69 | 0.31 | 2.17 |
| Ki67, RAD51 | 9.65E−03 | 0.25 | 0.95 | 0.71 | 0.73 | 0.27 | 2.67 |
| Ki67, PAR | 1.24E−02 | 0.30 | 0.94 | 0.75 | 0.68 | 0.31 | 2.36 |
| RAD51, PAR | 1.59E−02 | 0.47 | 0.88 | 0.69 | 0.74 | 0.27 | 2.63 |
| ERCC1, RAD51 | 1.97E−02 | 0.53 | 0.83 | 0.59 | 0.79 | 0.27 | 2.81 |
| PARP1, ATM | 2.02E−02 | 0.95 | 0.30 | 0.44 | 0.91 | 0.46 | 4.83 |
| PAR, ATM | 2.06E−02 | 0.32 | 0.88 | 0.67 | 0.64 | 0.36 | 1.85 |
| ERCC1, PAR | 2.11E−02 | 1.00 | 0.19 | 0.43 | 1.00 | 0.50 | |
| RAD51, PARP1 | 2.31E−02 | 0.50 | 0.80 | 0.56 | 0.77 | 0.30 | 2.39 |
| RAD51, MLH1 | 3.27E−02 | 0.70 | 0.58 | 0.45 | 0.79 | 0.38 | 2.18 |
| Ki67, ATM | 3.46E−02 | 0.68 | 0.56 | 0.46 | 0.76 | 0.40 | 1.93 |
| ERCC1, PARP1 | 3.58E−02 | 0.95 | 0.25 | 0.40 | 0.91 | 0.51 | 4.40 |
| Ki67, ERCC1 | 4.27E−02 | 1.00 | 0.20 | 0.39 | 1.00 | 0.53 | |
| Ki67, PARP1 | 5.86E−02 | 0.50 | 0.71 | 0.48 | 0.73 | 0.37 | 1.74 |
| PAR, PARP1 | 6.33E−02 | 0.95 | 0.19 | 0.42 | 0.86 | 0.52 | 2.96 |

TABLE 6
Three Marker Partition Analysis TNBCMARKERS

| Markers | pval | Sens | Spec | PosPow | NegPow | AER | RelRisk |
|---|---|---|---|---|---|---|---|
| NQ01, XPF, PMK2 | 2.34E−05 | 0.47 | 0.92 | 0.75 | 0.7727 | 0.2 | 3.3 |
| FANCD2, XPF, PMK2 | 2.94E−05 | 0.71 | 0.79 | 0.65 | 0.84 | 0.23 | 4.02 |
| NQ01, BRCA1, PMK2 | 4.50E−05 | 0.32 | 0.95 | 0.75 | 0.7292 | 0.3 | 2.7692 |
| FANCD2, PAR, XPF | 8.37E−05 | 0.65 | 0.80 | 0.63 | 0.82 | 0.25 | 3.44 |
| NQ01, PAR, PMK2 | 8.97E−05 | 0.21 | 0.96 | 0.8 | 0.6429 | 0.3 | 2.24 |
| BRCA1, PAR, RAD51 | 1.03E−04 | 0.37 | 0.98 | 0.88 | 0.77 | 0.22 | 3.79 |
| BRCA1, FANCD2, PMK2 | 1.08E−04 | 0.55 | 0.84 | 0.65 | 0.78 | 0.26 | 2.88 |
| BRCA1, XPF, PMK2 | 1.14E−04 | 0.55 | 0.84 | 0.65 | 0.78 | 0.26 | 2.88 |
| BRCA1, FANCD2, XPF | 1.39E−04 | 0.52 | 0.85 | 0.65 | 0.78 | 0.26 | 2.91 |
| ATM, FANCD2, XPF | 1.61E−04 | 0.70 | 0.78 | 0.62 | 0.83 | 0.25 | 3.69 |
| NQ01, BRCA1, PAR | 1.80E−04 | 0.55 | 0.77 | 0.6111 | 0.7273 | 0.3 | 2.2407 |
| ATM, PAR, XPF | 2.75E−04 | 0.35 | 0.96 | 0.80 | 0.74 | 0.25 | 3.09 |
| MLH1, PAR, XPF | 2.75E−04 | 0.35 | 0.96 | 0.80 | 0.74 | 0.25 | 3.09 |
| BRCA1, PAR, XPF | 2.92E−04 | 0.43 | 0.90 | 0.69 | 0.76 | 0.26 | 2.83 |
| NQ01, FANCD2, PMK2 | 3.11E−04 | 0.78 | 0.69 | 0.5833 | 0.8462 | 0.3 | 3.7917 |
| BRCA1, FANCD2, PAR | 3.19E−04 | 0.48 | 0.85 | 0.63 | 0.76 | 0.27 | 2.61 |
| NQ01, XPF, FANCD2 | 3.30E−04 | 0.72 | 0.79 | 0.65 | 0.8438 | 0.2 | 4.16 |
| BRCA1, RAD51, PMK2 | 4.21E−04 | 0.37 | 0.95 | 0.78 | 0.74 | 0.25 | 3.05 |
| FANCD2, MLH1, XPF | 4.30E−04 | 0.70 | 0.76 | 0.59 | 0.83 | 0.26 | 3.47 |
| BRCA1, MLH1, PMK2 | 4.33E−04 | 0.50 | 0.86 | 0.67 | 0.76 | 0.26 | 2.80 |
| PAR, RAD51, XPF | 4.56E−04 | 0.35 | 0.95 | 0.78 | 0.76 | 0.24 | 3.23 |
| NQ01, XPF, PAR | 5.18E−04 | 0.65 | 0.7 | 0.5909 | 0.75 | 0.3 | 2.3636 |
| NQ01, PAR, FANCD2 | 5.58E−04 | 0.83 | 0.61 | 0.5769 | 0.85 | 0.3 | 3.8462 |
| ATM, FANCD2, PMK2 | 6.23E−04 | 0.62 | 0.82 | 0.65 | 0.80 | 0.25 | 3.25 |
| NQ01, RAD51, FANCD2 | 6.44E−04 | 0.78 | 0.68 | 0.56 | 0.8519 | 0.3 | 3.78 |
| FANCD2, PAR, PMK2 | 6.91E−04 | 0.71 | 0.67 | 0.54 | 0.81 | 0.32 | 2.86 |
| BRCA1, PAR, PMK2 | 6.95E−04 | 0.55 | 0.81 | 0.61 | 0.77 | 0.28 | 2.65 |
| BRCA1, MLH1, XPF | 7.08E−04 | 0.52 | 0.85 | 0.65 | 0.78 | 0.26 | 2.91 |
| ATM, MLH1, PAR | 7.53E−04 | 0.43 | 0.91 | 0.71 | 0.76 | 0.25 | 2.97 |
| ATM, FANCD2, PAR | 7.84E−04 | 0.57 | 0.80 | 0.59 | 0.78 | 0.28 | 2.72 |
| FANCD2, MLH1, PAR | 7.84E−04 | 0.57 | 0.80 | 0.59 | 0.78 | 0.28 | 2.72 |
| FANCD2, RAD51, XPF | 8.39E−04 | 0.70 | 0.74 | 0.56 | 0.84 | 0.27 | 3.55 |
| PAR, XPF, PMK2 | 8.97E−04 | 0.33 | 0.95 | 0.78 | 0.73 | 0.27 | 2.83 |
| NQ01, BRCA1, FANCD2 | 9.62E−04 | 0.61 | 0.79 | 0.6111 | 0.7941 | 0.3 | 2.9683 |
| ATM, PAR, PMK2 | 9.66E−04 | 0.71 | 0.72 | 0.58 | 0.82 | 0.28 | 3.27 |
| ATM, XPF, PMK2 | 9.70E−04 | 0.29 | 0.97 | 0.86 | 0.72 | 0.27 | 3.03 |
| MLH1, XPF, PMK2 | 9.70E−04 | 0.29 | 0.97 | 0.86 | 0.72 | 0.27 | 3.03 |
| FANCD2, MLH1, PMK2 | 9.98E−04 | 0.76 | 0.64 | 0.53 | 0.83 | 0.32 | 3.20 |
| BRCA1, FANCD2, MLH1 | 1.02E−03 | 0.52 | 0.83 | 0.61 | 0.77 | 0.27 | 2.69 |
| ATM, BRCA1, FANCD2 | 1.02E−03 | 0.52 | 0.83 | 0.61 | 0.77 | 0.27 | 2.69 |
| FANCD2, RAD51, PMK2 | 1.03E−03 | 0.74 | 0.67 | 0.52 | 0.84 | 0.31 | 3.21 |
| NQ01, FANCD2, ATM | 1.19E−03 | 0.82 | 0.61 | 0.56 | 0.85 | 0.3 | 3.7333 |
| MLH1, PAR, RAD51 | 1.37E−03 | 0.45 | 0.88 | 0.64 | 0.78 | 0.25 | 2.86 |
| BRCA1, FANCD2, RAD51 | 1.40E−03 | 0.42 | 0.93 | 0.73 | 0.78 | 0.23 | 3.24 |
| FANCD2, PAR, RAD51 | 1.48E−03 | 0.70 | 0.72 | 0.54 | 0.84 | 0.29 | 3.32 |
| BRCA1, RAD51, XPF | 1.63E−03 | 0.42 | 0.93 | 0.73 | 0.78 | 0.23 | 3.24 |
| BRCA1, MLH1, RAD51 | 1.68E−03 | 0.42 | 0.93 | 0.73 | 0.78 | 0.23 | 3.24 |
| MLH1, PAR, PMK2 | 1.98E−03 | 0.43 | 0.87 | 0.64 | 0.74 | 0.28 | 2.46 |
| NQ01, MLH1, PAR | 2.02E−03 | 0.6 | 0.67 | 0.5455 | 0.7143 | 0.4 | 1.9091 |
| ATM, MLH1, PMK2 | 2.20E−03 | 0.71 | 0.69 | 0.56 | 0.82 | 0.30 | 3.06 |
| ATM, BRCA1, PMK2 | 2.25E−03 | 0.50 | 0.86 | 0.67 | 0.76 | 0.26 | 2.80 |
| ATM, FANCD2, MLH1 | 2.74E−03 | 0.74 | 0.64 | 0.52 | 0.83 | 0.32 | 3.01 |
| ATM, FANCD2, RAD51 | 2.82E−03 | 0.75 | 0.65 | 0.50 | 0.85 | 0.32 | 3.30 |
| ATM, BRCA1, XPF | 2.87E−03 | 0.57 | 0.78 | 0.57 | 0.78 | 0.29 | 2.60 |
| BRCA1, MLH1, PAR | 2.88E−03 | 0.43 | 0.85 | 0.60 | 0.74 | 0.29 | 2.35 |
| NQ01, BRCA1, XPF | 2.88E−03 | 0.5 | 0.79 | 0.5556 | 0.7561 | 0.3 | 2.2778 |
| NQ01, PMK2, ATM | 3.67E−03 | 0.39 | 0.87 | 0.6364 | 0.7027 | 0.3 | 2.1405 |
| ATM, BRCA1, PAR | 3.85E−03 | 0.43 | 0.88 | 0.64 | 0.75 | 0.27 | 2.57 |
| NQ01, MLH1, FANCD2 | 3.85E−03 | 0.78 | 0.65 | 0.5385 | 0.8462 | 0.3 | 3.5 |
| NQ01, BRCA1, RAD51 | 3.89E−03 | 0.26 | 0.95 | 0.7143 | 0.7308 | 0.3 | 2.6531 |
| NQ01, MLH1, PMK2 | 4.37E−03 | 0.53 | 0.81 | 0.5882 | 0.7632 | 0.3 | 2.4837 |
| ATM, RAD51, PMK2 | 4.61E−03 | 0.68 | 0.69 | 0.52 | 0.82 | 0.31 | 2.86 |
| ATM, MLH1, XPF | 5.04E−03 | 0.30 | 0.96 | 0.78 | 0.73 | 0.26 | 2.87 |
| NQ01, RAD51, PMK2 | 5.31E−03 | 0.79 | 0.62 | 0.5172 | 0.8519 | 0.3 | 3.4914 |
| FANCD2, MLH1, RAD51 | 5.41E−03 | 0.75 | 0.63 | 0.48 | 0.84 | 0.33 | 3.10 |
| ATM, BRCA1, MLH1 | 6.92E−03 | 0.52 | 0.78 | 0.55 | 0.76 | 0.31 | 2.31 |
| PAR, RAD51, PMK2 | 6.96E−03 | 0.37 | 0.92 | 0.70 | 0.75 | 0.26 | 2.80 |
| NQ01, XPF, ATM | 7.22E−03 | 0.58 | 0.75 | 0.5789 | 0.75 | 0.3 | 2.3158 |
| NQ01, RAD51, XPF | 8.83E−03 | 0.32 | 0.92 | 0.6667 | 0.7347 | 0.3 | 2.5128 |
| NQ01, PAR, ATM | 8.90E−03 | 0.95 | 0.32 | 0.5143 | 0.8889 | 0.4 | 4.6286 |
| NQ01, BRCA1, ATM | 9.26E−03 | 0.26 | 0.94 | 0.7143 | 0.6818 | 0.3 | 2.2449 |
| NQ01, BRCA1, MLH1 | 9.26E−03 | 0.25 | 0.95 | 0.7143 | 0.7059 | 0.3 | 2.4286 |
| MLH1, RAD51, PMK2 | 1.28E−02 | 0.53 | 0.77 | 0.53 | 0.77 | 0.31 | 2.28 |
| NQ01, XPF, MLH1 | 1.38E−02 | 0.7 | 0.58 | 0.4667 | 0.7857 | 0.4 | 2.1778 |
| MLH1, RAD51, XPF | 1.59E−02 | 0.45 | 0.84 | 0.56 | 0.77 | 0.29 | 2.40 |
| ATM, BRCA1, RAD51 | 1.62E−02 | 0.47 | 0.88 | 0.64 | 0.78 | 0.25 | 2.96 |
| NQ01, MLH1, ATM | 1.62E−02 | 0.74 | 0.58 | 0.5185 | 0.7826 | 0.4 | 2.3852 |
| ATM, PAR, RAD51 | 1.75E−02 | 0.60 | 0.72 | 0.50 | 0.79 | 0.32 | 2.44 |
| NQ01, RAD51, PAR | 1.77E−02 | 1 | 0.23 | 0.4419 | 1 | 0.5 | |
| ATM, RAD51, XPF | 1.81E−02 | 0.25 | 0.95 | 0.71 | 0.73 | 0.27 | 2.67 |
| NQ01, RAD51, MLH1 | 3.02E−02 | 0.58 | 0.76 | 0.55 | 0.7838 | 0.3 | 2.5438 |
| RAD51, XPF, PMK2 | 3.25E−02 | 0.53 | 0.79 | 0.56 | 0.78 | 0.29 | 2.47 |
| ATM, MLH1, RAD51 | 4.07E−02 | 0.40 | 0.86 | 0.57 | 0.76 | 0.29 | 2.33 |
| NQ01, RAD51, ATM | 5.47E−02 | 0.56 | 0.75 | 0.5556 | 0.75 | 0.3 | 2.2222 |

TABLE 7
Four Marker Partition Analysis TNBCMARKERS

| Markers | pval | Sens | Spec | PosPow | NegPow | AER | RelRisk |
|---|---|---|---|---|---|---|---|
| BRCA1, RAD51, PAR, PMK2 | 2.94E−10 | 0.42 | 1.00 | 1.00 | 0.72 | 0.23 | 3.55 |
| BRCA1, PAR, FANCD2, PMK2 | 1.95E−08 | 0.56 | 0.88 | 0.77 | 0.74 | 0.25 | 2.98 |
| BRCA1, FANCD2, PMK2, ATM | 3.40E−07 | 0.59 | 0.92 | 0.83 | 0.77 | 0.21 | 3.69 |
| BRCA1, RAD51, XPF, PMK2 | 5.82E−07 | 0.37 | 1.00 | 1.00 | 0.76 | 0.21 | 4.08 |
| RAD51, XPF, MLH1, PMK2 | 5.82E−07 | 0.35 | 1.00 | 1.00 | 0.75 | 0.22 | 3.92 |
| RAD51, XPF, PMK2, ATM | 5.82E−07 | 0.39 | 1.00 | 1.00 | 0.74 | 0.22 | 3.91 |
| BRCA1, RAD51, MLH1, PMK2 | 9.19E−07 | 0.32 | 1.00 | 1.00 | 0.73 | 0.24 | 3.77 |
| RAD51, XPF, FANCD2, PMK2 | 1.14E−06 | 0.47 | 0.94 | 0.82 | 0.76 | 0.23 | 3.44 |
| XPF, MLH1, FANCD2, PMK2 | 1.14E−06 | 0.47 | 0.94 | 0.82 | 0.76 | 0.23 | 3.44 |
| XPF, PAR, FANCD2, PMK2 | 1.14E−06 | 0.50 | 0.93 | 0.82 | 0.74 | 0.24 | 3.09 |
| BRCA1, MLH1, PAR, PMK2 | 1.20E−06 | 0.42 | 0.89 | 0.73 | 0.69 | 0.30 | 2.38 |
| BRCA1, RAD51, PMK2, ATM | 1.25E−06 | 0.33 | 1.00 | 1.00 | 0.71 | 0.25 | 3.50 |
| BRCA1, XPF, PAR, PMK2 | 1.37E−06 | 0.47 | 0.93 | 0.82 | 0.72 | 0.26 | 2.95 |
| RAD51, XPF, PAR, PMK2 | 1.37E−06 | 0.47 | 0.93 | 0.82 | 0.73 | 0.25 | 3.03 |
| XPF, MLH1, PAR, PMK2 | 1.37E−06 | 0.47 | 0.93 | 0.82 | 0.73 | 0.25 | 3.03 |
| BRCA1, XPF, FANCD2, PMK2 | 1.42E−06 | 0.53 | 0.94 | 0.83 | 0.77 | 0.22 | 3.61 |
| BRCA1, RAD51, FANCD2, PMK2 | 2.48E−06 | 0.44 | 0.97 | 0.89 | 0.76 | 0.22 | 3.64 |
| BRCA1, XPF, PMK2, ATM | 3.79E−06 | 0.44 | 0.97 | 0.89 | 0.74 | 0.23 | 3.47 |
| BRCA1, MLH1, FANCD2, PMK2 | 4.06E−06 | 0.56 | 0.88 | 0.71 | 0.78 | 0.24 | 3.21 |
| XPF, PAR, PMK2, ATM | 7.74E−06 | 0.44 | 0.96 | 0.89 | 0.70 | 0.26 | 2.93 |
| BRCA1, RAD51, MLH1, PAR | 1.01E−05 | 0.47 | 0.93 | 0.82 | 0.74 | 0.24 | 3.11 |
| BRCA1, RAD51, PAR, FANCD2 | 1.01E−05 | 0.50 | 0.93 | 0.82 | 0.74 | 0.24 | 3.18 |
| BRCA1, XPF, PAR, FANCD2 | 1.05E−05 | 0.67 | 0.82 | 0.71 | 0.79 | 0.24 | 3.41 |
| BRCA1, PAR, PMK2, ATM | 1.09E−05 | 0.61 | 0.87 | 0.79 | 0.74 | 0.24 | 3.03 |
| XPF, FANCD2, PMK2, ATM | 1.73E−05 | 0.47 | 0.96 | 0.89 | 0.75 | 0.22 | 3.56 |
| RAD51, FANCD2, PMK2, ATM | 1.73E−05 | 0.41 | 0.96 | 0.88 | 0.73 | 0.24 | 3.24 |
| BRCA1, PAR, FANCD2, ATM | 1.85E−05 | 0.59 | 0.87 | 0.77 | 0.74 | 0.25 | 2.97 |
| XPF, MLH1, PMK2, ATM | 2.02E−05 | 0.44 | 0.97 | 0.89 | 0.75 | 0.22 | 3.56 |
| BRCA1, XPF, MLH1, PMK2 | 2.34E−05 | 0.47 | 0.92 | 0.75 | 0.77 | 0.24 | 3.23 |
| BRCA1, MLH1, PAR, FANCD2 | 2.77E−05 | 0.56 | 0.82 | 0.67 | 0.74 | 0.28 | 2.58 |
| BRCA1, MLH1, PMK2, ATM | 7.41E−05 | 0.44 | 0.93 | 0.80 | 0.73 | 0.26 | 2.96 |
| RAD51, PAR, FANCD2, PMK2 | 7.48E−05 | 0.61 | 0.81 | 0.69 | 0.76 | 0.27 | 2.85 |
| BRCA1, XPF, FANCD2, ATM | 1.01E−04 | 0.59 | 0.86 | 0.71 | 0.77 | 0.24 | 3.16 |
| BRCA1, XPF, MLH1, PAR | 1.04E−04 | 0.45 | 0.87 | 0.69 | 0.70 | 0.30 | 2.33 |
| BRCA1, RAD51, XPF, PAR | 1.19E−04 | 0.47 | 0.90 | 0.75 | 0.73 | 0.27 | 2.78 |
| PAR, FANCD2, PMK2, ATM | 1.29E−04 | 0.71 | 0.73 | 0.67 | 0.76 | 0.28 | 2.80 |
| RAD51, XPF, PAR, FANCD2 | 1.57E−04 | 0.67 | 0.79 | 0.67 | 0.79 | 0.26 | 3.22 |
| XPF, MLH1, PAR, FANCD2 | 1.57E−04 | 0.67 | 0.79 | 0.67 | 0.79 | 0.26 | 3.22 |
| BRCA1, RAD51, PAR, ATM | 1.61E−04 | 0.50 | 0.88 | 0.75 | 0.71 | 0.28 | 2.58 |
| RAD51, MLH1, PAR, PMK2 | 2.10E−04 | 0.53 | 0.79 | 0.63 | 0.72 | 0.31 | 2.22 |
| BRCA1, XPF, PAR, ATM | 2.15E−04 | 0.58 | 0.76 | 0.65 | 0.70 | 0.32 | 2.18 |
| BRCA1, RAD51, XPF, FANCD2 | 3.29E−04 | 0.78 | 0.71 | 0.58 | 0.86 | 0.27 | 4.08 |
| XPF, PAR, FANCD2, ATM | 3.59E−04 | 0.71 | 0.75 | 0.67 | 0.78 | 0.27 | 3.07 |
| RAD51, MLH1, FANCD2, PMK2 | 3.62E−04 | 0.79 | 0.71 | 0.60 | 0.86 | 0.26 | 4.20 |
| BRCA1, RAD51, XPF, MLH1 | 3.94E−04 | 0.47 | 0.92 | 0.75 | 0.78 | 0.23 | 3.38 |
| RAD51, PAR, PMK2, ATM | 4.17E−04 | 0.44 | 0.88 | 0.73 | 0.68 | 0.31 | 2.25 |
| MLH1, FANCD2, PMK2, ATM | 4.23E−04 | 0.53 | 0.89 | 0.75 | 0.76 | 0.24 | 3.09 |
| MLH1, PAR, FANCD2, PMK2 | 4.86E−04 | 0.67 | 0.74 | 0.63 | 0.77 | 0.29 | 2.74 |
| BRCA1, MLH1, PAR, ATM | 5.12E−04 | 0.58 | 0.76 | 0.65 | 0.70 | 0.32 | 2.18 |
| RAD51, MLH1, PAR, FANCD2 | 5.79E−04 | 0.78 | 0.66 | 0.58 | 0.83 | 0.30 | 3.35 |
| MLH1, PAR, PMK2, ATM | 6.37E−04 | 0.61 | 0.75 | 0.65 | 0.72 | 0.31 | 2.31 |
| RAD51, XPF, MLH1, FANCD2 | 9.01E−04 | 0.79 | 0.69 | 0.58 | 0.86 | 0.27 | 4.18 |
| XPF, MLH1, PAR, ATM | 1.03E−03 | 0.58 | 0.77 | 0.65 | 0.71 | 0.31 | 2.26 |
| BRCA1, XPF, MLH1, FANCD2 | 1.21E−03 | 0.72 | 0.74 | 0.59 | 0.83 | 0.27 | 3.55 |
| RAD51, MLH1, PMK2, ATM | 1.24E−03 | 0.33 | 0.94 | 0.75 | 0.71 | 0.29 | 2.56 |
| MLH1, PAR, FANCD2, ATM | 1.37E−03 | 0.82 | 0.63 | 0.61 | 0.83 | 0.29 | 3.65 |
| RAD51, XPF, PAR, ATM | 1.60E−03 | 0.61 | 0.73 | 0.61 | 0.73 | 0.32 | 2.27 |
| BRCA1, MLH1, FANCD2, ATM | 1.79E−03 | 0.65 | 0.79 | 0.65 | 0.79 | 0.27 | 3.02 |
| RAD51, XPF, MLH1, PAR | 1.97E−03 | 0.63 | 0.71 | 0.57 | 0.76 | 0.32 | 2.37 |
| RAD51, XPF, FANCD2, ATM | 2.09E−03 | 0.76 | 0.70 | 0.59 | 0.84 | 0.28 | 3.69 |
| BRCA1, RAD51, XPF, ATM | 2.26E−03 | 0.50 | 0.88 | 0.69 | 0.76 | 0.26 | 2.85 |
| BRCA1, RAD51, MLH1, FANCD2 | 2.60E−03 | 0.78 | 0.68 | 0.56 | 0.85 | 0.29 | 3.78 |
| RAD51, PAR, FANCD2, ATM | 2.79E−03 | 0.24 | 0.96 | 0.80 | 0.64 | 0.34 | 2.22 |
| BRCA1, XPF, MLH1, ATM | 3.13E−03 | 0.58 | 0.77 | 0.61 | 0.75 | 0.30 | 2.44 |
| XPF, MLH1, FANCD2, ATM | 3.17E−03 | 0.76 | 0.70 | 0.59 | 0.84 | 0.28 | 3.69 |
| BRCA1, RAD51, FANCD2, ATM | 3.51E−03 | 0.53 | 0.86 | 0.69 | 0.75 | 0.27 | 2.77 |
| BRCA1, RAD51, MLH1, ATM | 3.51E−03 | 0.50 | 0.87 | 0.69 | 0.75 | 0.27 | 2.77 |
| RAD51, MLH1, FANCD2, ATM | 7.38E−03 | 0.76 | 0.67 | 0.57 | 0.83 | 0.30 | 3.39 |
| RAD51, XPF, MLH1, ATM | 1.10E−02 | 0.61 | 0.76 | 0.58 | 0.78 | 0.29 | 2.65 |
| RAD51, MLH1, PAR, ATM | 1.77E−02 | 0.28 | 0.88 | 0.63 | 0.64 | 0.36 | 1.73 |

TABLE 8
One Marker Probability Analysis TNBCMARKERS

| Markers | pval | AUC | Sens | Spec | PosPow | NegPow | AER | Frac.called | RelRisk |
|---|---|---|---|---|---|---|---|---|---|
| XPF | 9.31E−06 | 0.69 | 0.34 | 0.41 | 0.67 | 0.90 | 0.18 | 0.47 | 6.89 |
| FANCD2 | 3.22E−03 | 0.70 | 0.74 | 0.32 | 0.49 | 0.90 | 0.39 | 0.78 | 5.15 |
| ERCC1 | na | 0.57 | 0.00 | 0.15 | 0.00 | 0.77 | 1.00 | 0.13 | na |
| NQ01 | na | 0.56 | 0.00 | 0.22 | 0.00 | 0.78 | 1.00 | 0.18 | na |
| RAD51 | na | 0.55 | 0.00 | 0.00 | 0.00 | 0.00 | 1.00 | 0.01 | na |
| BRCA1 | na | 0.60 | 0.09 | 0.00 | 0.60 | 0.00 | 1.00 | 0.05 | na |
| MLH1 | na | 0.68 | 0.00 | 0.17 | 0.00 | 0.73 | 1.00 | 0.15 | na |
| PAR | na | 0.52 | 0.00 | 0.09 | 0.00 | 0.71 | 1.00 | 0.08 | na |
| PMK2 | na | 0.61 | 0.00 | 0.15 | 0.00 | 0.77 | 1.00 | 0.13 | na |
| PARP1 | na | 0.59 | 0.00 | 0.03 | 0.00 | 0.67 | 1.00 | 0.03 | na |
| ATM | na | 0.53 | 0.00 | 0.12 | 0.00 | 0.78 | 1.00 | 0.10 | na |

na = not applicable

TABLE 9
Two Marker Probability Analysis TNBCMARKERS

| Markers | pval | AUC | Sens | Spec | PosPow | NegPow | AER | Frac.called | RelRisk |
|---|---|---|---|---|---|---|---|---|---|
| RAD51; MLH1 | 5.18E−05 | 0.69 | 0.06 | 0.14 | 1.00 | 0.75 | 0.21 | 0.14 | 4.00 |
| NQO1; FANCD2 | 2.90E−04 | 0.70 | 0.72 | 0.45 | 0.53 | 0.86 | 0.33 | 0.82 | 3.88 |
| XPF; PARP1 | 3.80E−04 | 0.67 | 0.37 | 0.32 | 0.68 | 0.81 | 0.24 | 0.45 | 3.56 |
| XPF; MLH1 | 5.11E−04 | 0.70 | 0.41 | 0.50 | 0.61 | 0.79 | 0.28 | 0.65 | 2.84 |
| BRCA1; XPF | 5.22E−04 | 0.71 | 0.32 | 0.56 | 0.58 | 0.85 | 0.23 | 0.62 | 3.96 |
| XPF; PMK2 | 5.79E−04 | 0.76 | 0.41 | 0.17 | 0.74 | 0.92 | 0.19 | 0.32 | 8.84 |
| ERCC1; XPF | 7.62E−04 | 0.71 | 0.36 | 0.28 | 0.67 | 0.86 | 0.23 | 0.40 | 4.67 |
| NQO1; RAD51 | 8.28E−04 | 0.61 | 0.09 | 0.22 | 0.75 | 0.78 | 0.23 | 0.23 | 3.38 |
| FANCD2; PMK2 | 8.73E−04 | 0.74 | 0.66 | 0.43 | 0.52 | 0.86 | 0.35 | 0.79 | 3.66 |
| NQO1; XPF | 1.46E−03 | 0.72 | 0.36 | 0.24 | 0.60 | 0.88 | 0.27 | 0.39 | 5.10 |
| RAD51; XPF | 1.74E−03 | 0.67 | 0.33 | 0.39 | 0.65 | 0.79 | 0.26 | 0.50 | 3.05 |
| MLH1; FANCD2 | 3.93E−03 | 0.72 | 0.76 | 0.38 | 0.56 | 0.81 | 0.35 | 0.79 | 3.00 |
| BRCA1; FANCD2 | 3.99E−03 | 0.70 | 0.45 | 0.34 | 0.56 | 0.86 | 0.31 | 0.55 | 4.07 |
| FANCD2; ATM | 4.06E−03 | 0.65 | 0.69 | 0.35 | 0.50 | 0.90 | 0.37 | 0.74 | 5.00 |
| PAR; FANCD2 | 4.12E−03 | 0.69 | 0.64 | 0.38 | 0.49 | 0.90 | 0.37 | 0.75 | 4.86 |
| XPF; FANCD2 | 4.39E−03 | 0.73 | 0.55 | 0.42 | 0.53 | 0.83 | 0.33 | 0.69 | 3.18 |
| MLH1; PMK2 | 4.57E−03 | 0.69 | 0.50 | 0.21 | 0.53 | 0.93 | 0.35 | 0.48 | 7.44 |
| RAD51; PAR | 4.82E−03 | 0.58 | 0.10 | 0.09 | 1.00 | 0.71 | 0.20 | 0.12 | 3.50 |
| ERCC1; FANCD2 | 6.05E−03 | 0.67 | 0.66 | 0.38 | 0.49 | 0.85 | 0.38 | 0.77 | 3.17 |
| FANCD2; PARP1 | 6.17E−03 | 0.69 | 0.58 | 0.31 | 0.48 | 0.90 | 0.38 | 0.66 | 4.75 |
| NQO1; MLH1 | 7.78E−03 | 0.65 | 0.48 | 0.23 | 0.53 | 0.82 | 0.36 | 0.49 | 3.02 |
| XPF; ATM | 9.34E−03 | 0.64 | 0.32 | 0.28 | 0.63 | 0.80 | 0.28 | 0.40 | 3.13 |
| RAD51; FANCD2 | 1.14E−02 | 0.70 | 0.48 | 0.32 | 0.48 | 0.86 | 0.36 | 0.59 | 3.56 |
| MLH1; PARP1 | 1.16E−02 | 0.67 | 0.15 | 0.23 | 0.83 | 0.78 | 0.21 | 0.25 | 3.75 |
| ERCC1; MLH1 | 1.39E−02 | 0.66 | 0.34 | 0.26 | 0.48 | 0.89 | 0.34 | 0.44 | 4.30 |
| BRCA1; PAR | 1.89E−02 | 0.59 | 0.17 | 0.12 | 0.71 | 0.86 | 0.21 | 0.17 | 5.00 |
| NQO1; BRCA1 | 1.94E−02 | 0.62 | 0.21 | 0.22 | 0.54 | 0.78 | 0.32 | 0.32 | 2.42 |
| BRCA1; PMK2 | 3.08E−02 | 0.67 | 0.26 | 0.17 | 0.56 | 0.83 | 0.32 | 0.30 | 3.38 |
| ERCC1; NQO1 | 3.68E−02 | 0.59 | 0.67 | 0.29 | 0.44 | 0.78 | 0.45 | 0.76 | 2.02 |
| MLH1; ATM | 4.96E−02 | 0.66 | 0.42 | 0.23 | 0.52 | 0.76 | 0.38 | 0.48 | 2.21 |
| ERCC1; PMK2 | 6.63E−02 | 0.63 | 0.64 | 0.21 | 0.48 | 0.76 | 0.44 | 0.65 | 2.03 |
| XPF; PAR | 6.66E−02 | 0.65 | 0.37 | 0.17 | 0.73 | 0.60 | 0.33 | 0.37 | 1.83 |
| PMK2; ATM | 8.93E−02 | 0.63 | 0.73 | 0.20 | 0.46 | 0.79 | 0.47 | 0.74 | 2.14 |
| MLH1; PAR | 8.98E−02 | 0.64 | 0.17 | 0.10 | 0.63 | 0.83 | 0.29 | 0.17 | 3.75 |
| PMK2; PARP1 | 9.57E−02 | 0.62 | 0.53 | 0.15 | 0.49 | 0.75 | 0.45 | 0.52 | 1.95 |
| BRCA1; ATM | 1.10E−01 | 0.60 | 0.16 | 0.13 | 0.56 | 0.78 | 0.33 | 0.21 | 2.50 |
| NQO1; PMK2 | 1.20E−01 | 0.61 | 0.73 | 0.27 | 0.39 | 0.76 | 0.51 | 0.88 | 1.65 |
| NQO1; ATM | 1.28E−01 | 0.56 | 0.48 | 0.31 | 0.39 | 0.74 | 0.48 | 0.71 | 1.51 |
| PAR; PMK2 | 1.35E−01 | 0.59 | 0.59 | 0.18 | 0.40 | 0.82 | 0.51 | 0.68 | 2.23 |
| BRCA1; MLH1 | 1.43E−01 | 0.69 | 0.18 | 0.23 | 0.43 | 0.82 | 0.35 | 0.33 | 2.43 |
| NQO1; PAR | 2.14E−01 | 0.54 | 0.27 | 0.23 | 0.36 | 0.80 | 0.46 | 0.45 | 1.82 |
| RAD51; PMK2 | 2.21E−01 | 0.64 | 0.06 | 0.16 | 0.40 | 0.83 | 0.29 | 0.18 | 2.40 |
| ERCC1; BRCA1 | 2.53E−01 | 0.65 | 0.15 | 0.20 | 0.50 | 0.76 | 0.33 | 0.28 | 2.13 |
| BRCA1; PARP1 | 8.85E−01 | 0.62 | 0.09 | 0.02 | 0.50 | 0.50 | 0.50 | 0.08 | 1.00 |
| ERCC1; RAD51 | | 0.62 | | 0.12 | | 0.73 | | 0.11 | |
| ERCC1; PAR | | 0.62 | | 0.21 | | 0.85 | | 0.16 | |
| ERCC1; PARP1 | | 0.66 | | 0.15 | | 0.77 | | 0.13 | |
| ERCC1; ATM | | 0.58 | | 0.23 | | 0.76 | | 0.20 | |
| NQO1; PARP1 | | 0.57 | | 0.22 | | 0.78 | | 0.19 | |
| RAD51; BRCA1 | | 0.58 | 0.09 | | 0.75 | | | 0.04 | |
| RAD51; PARP1 | | 0.58 | 0.00 | 0.00 | 0.00 | 0.00 | 1.00 | 0.02 | 0.00 |
| RAD51; ATM | | 0.57 | | 0.12 | | 0.78 | | 0.10 | |
| PAR; PARP1 | | 0.52 | | 0.11 | | 0.75 | | 0.10 | |
| PAR; ATM | | 0.52 | | 0.15 | | 0.78 | | 0.12 | |
| PARP1; ATM | | 0.61 | | 0.12 | | 0.78 | | 0.10 | |

TABLE 10
Three Marker Probability Analysis TNBCMARKERS

| Markers | p val | AUC | Sens | Spec | PosPow | NegPow | AER | Frac.called | RelRisk |
|---|---|---|---|---|---|---|---|---|---|
| NQ01; XPF; FANCD2 | 9.24E−06 | 0.76 | 0.65 | 0.48 | 0.63 | 0.90 | 0.24 | 0.71 | 6.25 |
| ERCC1; RAD51; PAR | 2.06E−05 | 0.69 | 0.11 | 0.25 | 1.00 | 0.87 | 0.11 | 0.22 | 7.50 |
| XPF; FANCD2; PMK2 | 2.81E−05 | 0.79 | 0.67 | 0.43 | 0.67 | 0.86 | 0.25 | 0.69 | 4.67 |
| NQ01; XPF; PMK2 | 3.04E−05 | 0.76 | 0.47 | 0.32 | 0.71 | 0.86 | 0.21 | 0.47 | 5.24 |
| NQ01; MLH1; FANCD2 | 3.46E−05 | 0.71 | 0.71 | 0.50 | 0.55 | 0.90 | 0.30 | 0.82 | 5.68 |
| ERCC1; NQ01; XPF | 6.86E−05 | 0.74 | 0.47 | 0.37 | 0.68 | 0.85 | 0.22 | 0.52 | 4.60 |
| XPF; MLH1; PMK2 | 7.61E−05 | 0.78 | 0.42 | 0.37 | 0.70 | 0.82 | 0.23 | 0.51 | 3.92 |
| MLH1; FANCD2; PMK2 | 9.64E−05 | 0.75 | 0.70 | 0.47 | 0.55 | 0.90 | 0.31 | 0.81 | 5.29 |
| BRCA1; XPF; PMK2 | 1.50E−04 | 0.78 | 0.39 | 0.24 | 0.72 | 0.93 | 0.18 | 0.36 | 10.83 |
| NQ01; BRCA1; FANCD2 | 1.71E−04 | 0.71 | 0.66 | 0.47 | 0.53 | 0.90 | 0.32 | 0.79 | 5.08 |
| XPF; MLH1; FANCD2 | 2.17E−04 | 0.74 | 0.66 | 0.50 | 0.57 | 0.85 | 0.30 | 0.79 | 3.86 |
| BRCA1; XPF; MLH1 | 2.27E−04 | 0.71 | 0.39 | 0.59 | 0.62 | 0.80 | 0.26 | 0.70 | 3.10 |
| BRCA1; FANCD2; PMK2 | 2.74E−04 | 0.73 | 0.67 | 0.48 | 0.55 | 0.86 | 0.32 | 0.81 | 3.99 |
| ERCC1; MLH1; FANCD2 | 2.83E−04 | 0.70 | 0.77 | 0.46 | 0.52 | 0.90 | 0.33 | 0.85 | 5.05 |
| ERCC1; XPF; PMK2 | 2.94E−04 | 0.76 | 0.44 | 0.31 | 0.70 | 0.83 | 0.23 | 0.46 | 4.02 |
| ERCC1; RAD51; XPF | 3.15E−04 | 0.70 | 0.35 | 0.32 | 0.65 | 0.88 | 0.22 | 0.43 | 5.18 |
| XPF; MLH1; PARP1 | 3.25E−04 | 0.70 | 0.38 | 0.45 | 0.59 | 0.85 | 0.25 | 0.57 | 3.90 |
| NQ01; XPF; MLH1 | 3.41E−04 | 0.72 | 0.45 | 0.40 | 0.63 | 0.81 | 0.27 | 0.58 | 3.23 |
| RAD51; XPF; MLH1 | 3.90E−04 | 0.71 | 0.42 | 0.52 | 0.64 | 0.80 | 0.25 | 0.65 | 3.26 |
| ERCC1; XPF; MLH1 | 4.31E−04 | 0.73 | 0.41 | 0.48 | 0.62 | 0.81 | 0.26 | 0.62 | 3.27 |
| RAD51; PAR; ATM | 4.38E−04 | 0.56 | 0.07 | 0.15 | 1.00 | 0.78 | 0.18 | 0.15 | 4.50 |
| RAD51; FANCD2; PMK2 | 4.76E−04 | 0.75 | 0.67 | 0.45 | 0.52 | 0.86 | 0.34 | 0.80 | 3.80 |
| NQ01; RAD51; FANCD2 | 5.99E−04 | 0.71 | 0.66 | 0.45 | 0.51 | 0.86 | 0.34 | 0.80 | 3.71 |
| RAD51; XPF; PMK2 | 6.17E−04 | 0.75 | 0.33 | 0.19 | 0.69 | 0.92 | 0.21 | 0.30 | 8.94 |
| NQ01; FANCD2; PARP1 | 6.66E−04 | 0.69 | 0.71 | 0.44 | 0.54 | 0.83 | 0.34 | 0.81 | 3.11 |
| RAD51; XPF; PARP1 | 7.35E−04 | 0.65 | 0.36 | 0.32 | 0.67 | 0.81 | 0.25 | 0.45 | 3.47 |
| XPF; PAR; PMK2 | 8.54E−04 | 0.72 | 0.41 | 0.29 | 0.71 | 0.82 | 0.24 | 0.44 | 4.00 |
| BRCA1; PAR; FANCD2 | 9.00E−04 | 0.71 | 0.50 | 0.39 | 0.61 | 0.86 | 0.27 | 0.59 | 4.26 |
| FANCD2; PMK2; PARP1 | 9.27E−04 | 0.73 | 0.67 | 0.41 | 0.51 | 0.88 | 0.35 | 0.78 | 4.26 |
| ERCC1; NQ01; FANCD2 | 9.74E−04 | 0.69 | 0.74 | 0.44 | 0.53 | 0.80 | 0.36 | 0.85 | 2.67 |
| BRCA1; XPF; PARP1 | 9.81E−04 | 0.69 | 0.35 | 0.55 | 0.57 | 0.81 | 0.27 | 0.66 | 3.00 |
| XPF; PMK2; PARP1 | 1.12E−03 | 0.74 | 0.41 | 0.20 | 0.70 | 0.86 | 0.24 | 0.36 | 4.90 |
| XPF; MLH1; PAR | 1.18E−03 | 0.68 | 0.40 | 0.50 | 0.63 | 0.76 | 0.28 | 0.65 | 2.68 |
| ERCC1; XPF; PARP1 | 1.41E−03 | 0.70 | 0.36 | 0.30 | 0.67 | 0.79 | 0.26 | 0.43 | 3.20 |
| ERCC1; BRCA1; XPF | 1.43E−03 | 0.74 | 0.36 | 0.35 | 0.60 | 0.85 | 0.26 | 0.48 | 3.90 |
| BRCA1; MLH1; PMK2 | 1.44E−03 | 0.71 | 0.50 | 0.24 | 0.57 | 0.93 | 0.30 | 0.48 | 8.57 |
| MLH1; PMK2; PARP1 | 1.58E−03 | 0.69 | 0.58 | 0.22 | 0.59 | 0.93 | 0.30 | 0.50 | 8.31 |
| NQ01; RAD51; XPF | 1.73E−03 | 0.71 | 0.34 | 0.24 | 0.58 | 0.88 | 0.28 | 0.38 | 4.92 |
| NQ01; XPF; PARP1 | 1.81E−03 | 0.68 | 0.36 | 0.26 | 0.60 | 0.84 | 0.28 | 0.41 | 3.80 |
| ERCC1; XPF; FANCD2 | 1.87E−03 | 0.74 | 0.52 | 0.45 | 0.52 | 0.87 | 0.31 | 0.69 | 3.87 |
| RAD51; PAR; FANCD2 | 1.95E−03 | 0.74 | 0.54 | 0.35 | 0.58 | 0.89 | 0.29 | 0.59 | 5.48 |
| ERCC1; NQ01; BRCA1 | 1.97E−03 | 0.66 | 0.52 | 0.31 | 0.53 | 0.83 | 0.35 | 0.58 | 3.05 |
| XPF; PMK2; ATM | 2.16E−03 | 0.73 | 0.37 | 0.26 | 0.65 | 0.88 | 0.24 | 0.39 | 5.18 |
| PAR; FANCD2; PMK2 | 2.28E−03 | 0.70 | 0.64 | 0.42 | 0.51 | 0.86 | 0.35 | 0.78 | 3.77 |
| ERCC1; BRCA1; MLH1 | 2.29E−03 | 0.69 | 0.41 | 0.28 | 0.62 | 0.81 | 0.29 | 0.46 | 3.25 |
| ERCC1; NQ01; MLH1 | 2.34E−03 | 0.67 | 0.63 | 0.34 | 0.50 | 0.84 | 0.37 | 0.70 | 3.13 |
| NQ01; FANCD2; ATM | 2.42E−03 | 0.66 | 0.69 | 0.43 | 0.53 | 0.84 | 0.35 | 0.81 | 3.29 |
| XPF; PAR; FANCD2 | 2.75E−03 | 0.74 | 0.46 | 0.48 | 0.54 | 0.85 | 0.29 | 0.67 | 3.66 |
| RAD51; BRCA1; FANCD2 | 2.86E−03 | 0.69 | 0.50 | 0.34 | 0.55 | 0.86 | 0.31 | 0.58 | 4.05 |
| NQ01; XPF; ATM | 2.94E−03 | 0.67 | 0.35 | 0.36 | 0.55 | 0.83 | 0.30 | 0.51 | 3.30 |
| NQ01; FANCD2; PMK2 | 3.01E−03 | 0.72 | 0.72 | 0.43 | 0.51 | 0.79 | 0.38 | 0.87 | 2.47 |
| ERCC1; XPF; PAR | 3.02E−03 | 0.70 | 0.34 | 0.25 | 0.71 | 0.76 | 0.26 | 0.38 | 3.04 |
| BRCA1; XPF; PAR | 3.10E−03 | 0.67 | 0.33 | 0.46 | 0.63 | 0.77 | 0.28 | 0.58 | 2.68 |
| NQ01; PAR; FANCD2 | 3.15E−03 | 0.68 | 0.68 | 0.40 | 0.53 | 0.83 | 0.36 | 0.79 | 3.03 |
| RAD51; MLH1; FANCD2 | 3.19E−03 | 0.72 | 0.72 | 0.38 | 0.53 | 0.85 | 0.35 | 0.77 | 3.48 |
| MLH1; PAR; FANCD2 | 3.34E−03 | 0.70 | 0.75 | 0.44 | 0.53 | 0.84 | 0.35 | 0.86 | 3.28 |
| XPF; MLH1; ATM | 3.48E−03 | 0.68 | 0.39 | 0.48 | 0.60 | 0.79 | 0.28 | 0.62 | 2.91 |
| RAD51; XPF; FANCD2 | 3.93E−03 | 0.71 | 0.50 | 0.45 | 0.50 | 0.84 | 0.33 | 0.70 | 3.20 |
| ERCC1; BRCA1; PMK2 | 4.11E−03 | 0.68 | 0.52 | 0.26 | 0.61 | 0.79 | 0.32 | 0.52 | 2.88 |
| MLH1; FANCD2; ATM | 4.40E−03 | 0.69 | 0.69 | 0.41 | 0.50 | 0.88 | 0.36 | 0.80 | 4.00 |
| RAD51; BRCA1; XPF | 4.83E−03 | 0.69 | 0.31 | 0.52 | 0.56 | 0.80 | 0.27 | 0.62 | 2.85 |
| NQ01; BRCA1; XPF | 4.91E−03 | 0.73 | 0.36 | 0.26 | 0.60 | 0.80 | 0.30 | 0.42 | 3.00 |
| FANCD2; PARP1; ATM | 5.00E−03 | 0.66 | 0.62 | 0.35 | 0.49 | 0.90 | 0.37 | 0.71 | 4.86 |
| NQ01; MLH1; ATM | 5.12E−03 | 0.64 | 0.55 | 0.39 | 0.47 | 0.84 | 0.38 | 0.72 | 2.95 |
| RAD51; FANCD2; ATM | 5.16E−03 | 0.67 | 0.66 | 0.35 | 0.49 | 0.90 | 0.37 | 0.73 | 4.87 |
| ERCC1; FANCD2; PMK2 | 5.46E−03 | 0.70 | 0.66 | 0.41 | 0.53 | 0.79 | 0.37 | 0.79 | 2.45 |
| PAR; FANCD2; PARP1 | 5.60E−03 | 0.67 | 0.57 | 0.38 | 0.47 | 0.90 | 0.37 | 0.71 | 4.71 |
| XPF; PARP1; ATM | 5.71E−03 | 0.64 | 0.32 | 0.32 | 0.63 | 0.78 | 0.28 | 0.44 | 2.88 |
| FANCD2; PMK2; ATM | 5.94E−03 | 0.72 | 0.66 | 0.38 | 0.50 | 0.86 | 0.37 | 0.77 | 3.50 |
| BRCA1; MLH1; FANCD2 | 5.99E−03 | 0.71 | 0.65 | 0.40 | 0.54 | 0.81 | 0.34 | 0.74 | 2.92 |
| NQ01; RAD51; PARP1 | 6.17E−03 | 0.62 | 0.16 | 0.22 | 0.63 | 0.82 | 0.24 | 0.26 | 3.54 |
| RAD51; MLH1; PARP1 | 6.27E−03 | 0.68 | 0.15 | 0.21 | 0.83 | 0.81 | 0.18 | 0.23 | 4.44 |
| MLH1; FANCD2; PARP1 | 6.44E−03 | 0.71 | 0.75 | 0.38 | 0.55 | 0.81 | 0.36 | 0.80 | 2.84 |
| ERCC1; MLH1; PMK2 | 6.73E−03 | 0.69 | 0.58 | 0.29 | 0.51 | 0.85 | 0.36 | 0.61 | 3.43 |
| RAD51; MLH1; PMK2 | 6.83E−03 | 0.69 | 0.45 | 0.21 | 0.50 | 0.93 | 0.36 | 0.47 | 7.00 |
| RAD51; BRCA1; PAR | 7.11E−03 | 0.63 | 0.24 | 0.12 | 0.78 | 0.75 | 0.24 | 0.21 | 3.11 |
| NQ01; RAD51; MLH1 | 7.12E−03 | 0.67 | 0.50 | 0.24 | 0.52 | 0.83 | 0.37 | 0.52 | 3.10 |
| ERCC1; BRCA1; FANCD2 | 7.49E−03 | 0.69 | 0.56 | 0.40 | 0.47 | 0.85 | 0.38 | 0.74 | 3.08 |
| BRCA1; FANCD2; PARP1 | 8.08E−03 | 0.69 | 0.47 | 0.33 | 0.54 | 0.86 | 0.33 | 0.56 | 3.75 |
| NQ01; BRCA1; PARP1 | 8.13E−03 | 0.62 | 0.24 | 0.23 | 0.53 | 0.82 | 0.31 | 0.34 | 3.02 |
| NQ01; BRCA1; MLH1 | 8.29E−03 | 0.68 | 0.42 | 0.25 | 0.52 | 0.83 | 0.36 | 0.48 | 3.11 |
| RAD51; XPF; ATM | 8.49E−03 | 0.63 | 0.33 | 0.28 | 0.63 | 0.80 | 0.28 | 0.41 | 3.13 |
| MLH1; PAR; PMK2 | 8.88E−03 | 0.66 | 0.55 | 0.27 | 0.52 | 0.87 | 0.37 | 0.59 | 3.87 |
| MLH1; PARP1; ATM | 8.94E−03 | 0.66 | 0.42 | 0.31 | 0.57 | 0.81 | 0.32 | 0.51 | 2.97 |
| ERCC1; RAD51; FANCD2 | 9.04E−03 | 0.69 | 0.61 | 0.38 | 0.46 | 0.85 | 0.39 | 0.75 | 3.01 |
| XPF; FANCD2; PARP1 | 9.76E−03 | 0.71 | 0.52 | 0.41 | 0.50 | 0.83 | 0.35 | 0.69 | 2.90 |
| RAD51; PAR; PARP1 | 1.01E−02 | 0.56 | 0.10 | 0.08 | 1.00 | 0.67 | 0.22 | 0.11 | 3.00 |
| BRCA1; FANCD2; ATM | 1.04E−02 | 0.68 | 0.52 | 0.37 | 0.54 | 0.82 | 0.34 | 0.64 | 2.95 |
| ERCC1; FANCD2; PARP1 | 1.05E−02 | 0.68 | 0.68 | 0.35 | 0.49 | 0.83 | 0.39 | 0.76 | 2.93 |
| MLH1; PMK2; ATM | 1.07E−02 | 0.69 | 0.57 | 0.26 | 0.50 | 0.88 | 0.38 | 0.60 | 4.00 |
| RAD51; MLH1; PAR | 1.34E−02 | 0.69 | 0.14 | 0.12 | 0.80 | 0.75 | 0.23 | 0.16 | 3.20 |
| ERCC1; MLH1; PAR | 1.35E−02 | 0.64 | 0.45 | 0.25 | 0.57 | 0.81 | 0.33 | 0.48 | 3.01 |
| ERCC1; RAD51; MLH1 | 1.37E−02 | 0.69 | 0.32 | 0.24 | 0.50 | 0.88 | 0.32 | 0.40 | 4.25 |
| BRCA1; XPF; FANCD2 | 1.48E−02 | 0.72 | 0.47 | 0.45 | 0.52 | 0.81 | 0.33 | 0.68 | 2.67 |
| PAR; FANCD2; ATM | 1.51E−02 | 0.65 | 0.67 | 0.33 | 0.51 | 0.88 | 0.37 | 0.74 | 4.11 |
| MLH1; PAR; PARP1 | 1.57E−02 | 0.64 | 0.23 | 0.23 | 0.54 | 0.80 | 0.32 | 0.34 | 2.69 |
| BRCA1; PAR; PMK2 | 1.60E−02 | 0.64 | 0.55 | 0.23 | 0.53 | 0.85 | 0.37 | 0.57 | 3.47 |
| ERCC1; PAR; FANCD2 | 1.61E−02 | 0.69 | 0.67 | 0.35 | 0.51 | 0.81 | 0.38 | 0.75 | 2.70 |
| NQ01; RAD51; BRCA1 | 1.67E−02 | 0.65 | 0.27 | 0.22 | 0.53 | 0.78 | 0.34 | 0.36 | 2.38 |
| BRCA1; MLH1; ATM | 1.73E−02 | 0.67 | 0.35 | 0.28 | 0.55 | 0.79 | 0.33 | 0.46 | 2.61 |
| ERCC1; NQ01; PARP1 | 1.84E−02 | 0.64 | 0.63 | 0.31 | 0.43 | 0.83 | 0.44 | 0.74 | 2.45 |
| NQ01; MLH1; PARP1 | 1.92E−02 | 0.65 | 0.48 | 0.25 | 0.53 | 0.71 | 0.39 | 0.54 | 1.87 |
| ERCC1; NQ01; PMK2 | 2.07E−02 | 0.61 | 0.72 | 0.32 | 0.43 | 0.83 | 0.45 | 0.85 | 2.45 |
| RAD51; XPF; PAR | 2.14E−02 | 0.65 | 0.38 | 0.27 | 0.73 | 0.70 | 0.29 | 0.43 | 2.44 |
| BRCA1; MLH1; PARP1 | 2.17E−02 | 0.68 | 0.24 | 0.28 | 0.53 | 0.81 | 0.31 | 0.39 | 2.80 |
| RAD51; FANCD2; PARP1 | 2.22E−02 | 0.69 | 0.50 | 0.31 | 0.47 | 0.86 | 0.38 | 0.61 | 3.29 |
| ERCC1; NQ01; RAD51 | 2.45E−02 | 0.66 | 0.72 | 0.27 | 0.44 | 0.81 | 0.45 | 0.77 | 2.32 |
| NQ01; XPF; PAR | 2.45E−02 | 0.67 | 0.33 | 0.25 | 0.59 | 0.72 | 0.34 | 0.43 | 2.12 |
| NQ01; BRCA1; PAR | 2.48E−02 | 0.58 | 0.30 | 0.24 | 0.50 | 0.80 | 0.36 | 0.41 | 2.50 |
| NQ01; BRCA1; ATM | 2.55E−02 | 0.64 | 0.32 | 0.33 | 0.50 | 0.78 | 0.35 | 0.51 | 2.30 |
| XPF; PAR; PARP1 | 2.56E−02 | 0.63 | 0.33 | 0.25 | 0.71 | 0.65 | 0.32 | 0.41 | 2.04 |
| ERCC1; XPF; ATM | 2.66E−02 | 0.65 | 0.33 | 0.30 | 0.59 | 0.74 | 0.32 | 0.47 | 2.25 |
| BRCA1; XPF; ATM | 2.73E−02 | 0.65 | 0.32 | 0.33 | 0.56 | 0.78 | 0.32 | 0.48 | 2.56 |
| ERCC1; NQ01; ATM | 2.81E−02 | 0.62 | 0.60 | 0.37 | 0.42 | 0.80 | 0.44 | 0.81 | 2.09 |
| ERCC1; PARP1; ATM | 2.91E−02 | 0.61 | 0.17 | 0.25 | 0.63 | 0.74 | 0.30 | 0.31 | 2.38 |
| NQ01; MLH1; PMK2 | 2.98E−02 | 0.67 | 0.63 | 0.32 | 0.41 | 0.83 | 0.46 | 0.79 | 2.35 |
| ERCC1; FANCD2; ATM | 3.14E−02 | 0.65 | 0.54 | 0.36 | 0.43 | 0.86 | 0.41 | 0.72 | 3.00 |
| ERCC1; BRCA1; PAR | 3.20E−02 | 0.66 | 0.24 | 0.24 | 0.54 | 0.86 | 0.30 | 0.34 | 3.77 |
| ERCC1; MLH1; PARP1 | 3.24E−02 | 0.67 | 0.34 | 0.30 | 0.48 | 0.78 | 0.37 | 0.49 | 2.20 |
| BRCA1; PMK2; PARP1 | 3.35E−02 | 0.67 | 0.39 | 0.19 | 0.59 | 0.73 | 0.35 | 0.41 | 2.22 |
| BRCA1; PMK2; ATM | 3.35E−02 | 0.65 | 0.47 | 0.22 | 0.52 | 0.79 | 0.39 | 0.51 | 2.42 |
| NQ01; PMK2; PARP1 | 4.22E−02 | 0.61 | 0.78 | 0.29 | 0.41 | 0.81 | 0.49 | 0.90 | 2.15 |
| NQ01; PMK2; ATM | 4.28E−02 | 0.63 | 0.70 | 0.33 | 0.45 | 0.77 | 0.45 | 0.84 | 1.97 |
| PMK2; PARP1; ATM | 4.76E−02 | 0.66 | 0.63 | 0.23 | 0.48 | 0.80 | 0.44 | 0.66 | 2.37 |
| XPF; PAR; ATM | 4.95E−02 | 0.61 | 0.34 | 0.28 | 0.71 | 0.62 | 0.34 | 0.47 | 1.88 |
| ERCC1; NQ01; PAR | 5.00E−02 | 0.63 | 0.66 | 0.31 | 0.41 | 0.80 | 0.47 | 0.81 | 2.07 |
| NQ01; BRCA1; PMK2 | 5.09E−02 | 0.68 | 0.76 | 0.27 | 0.43 | 0.80 | 0.47 | 0.85 | 2.16 |
| RAD51; BRCA1; PMK2 | 5.14E−02 | 0.65 | 0.24 | 0.17 | 0.50 | 0.83 | 0.36 | 0.30 | 3.00 |
| BRCA1; PAR; ATM | 6.08E−02 | 0.58 | 0.17 | 0.16 | 0.63 | 0.78 | 0.29 | 0.23 | 2.81 |
| NQ01; RAD51; PAR | 6.21E−02 | 0.58 | 0.28 | 0.23 | 0.44 | 0.80 | 0.39 | 0.41 | 2.22 |
| ERCC1; BRCA1; ATM | 6.51E−02 | 0.63 | 0.23 | 0.26 | 0.50 | 0.78 | 0.34 | 0.38 | 2.25 |
| BRCA1; PAR; PARP1 | 6.52E−02 | 0.59 | 0.17 | 0.12 | 0.63 | 0.75 | 0.31 | 0.20 | 2.50 |
| RAD51; PAR; PMK2 | 6.57E−02 | 0.65 | 0.59 | 0.18 | 0.49 | 0.82 | 0.43 | 0.59 | 2.67 |
| NQ01; MLH1; PAR | 6.63E−02 | 0.61 | 0.40 | 0.24 | 0.44 | 0.80 | 0.43 | 0.52 | 2.22 |
| RAD51; BRCA1; ATM | 6.77E−02 | 0.60 | 0.17 | 0.13 | 0.63 | 0.78 | 0.29 | 0.20 | 2.81 |
| ERCC1; BRCA1; PARP1 | 6.89E−02 | 0.68 | 0.21 | 0.21 | 0.54 | 0.81 | 0.31 | 0.30 | 2.87 |
| RAD51; MLH1; ATM | 7.14E−02 | 0.67 | 0.37 | 0.23 | 0.50 | 0.76 | 0.38 | 0.45 | 2.13 |
| XPF; FANCD2; ATM | 7.70E−02 | 0.69 | 0.45 | 0.37 | 0.48 | 0.79 | 0.37 | 0.63 | 2.31 |
| NQ01; PARP1; ATM | 8.06E−02 | 0.61 | 0.45 | 0.31 | 0.42 | 0.74 | 0.45 | 0.65 | 1.63 |
| ERCC1; PAR; PMK2 | 8.60E−02 | 0.65 | 0.68 | 0.27 | 0.42 | 0.81 | 0.48 | 0.79 | 2.25 |
| PAR; PMK2; PARP1 | 8.88E−02 | 0.59 | 0.59 | 0.20 | 0.46 | 0.77 | 0.46 | 0.64 | 1.99 |
| NQ01; PAR; PARP1 | 9.14E−02 | 0.54 | 0.37 | 0.25 | 0.42 | 0.76 | 0.44 | 0.52 | 1.80 |
| ERCC1; MLH1; ATM | 9.44E−02 | 0.65 | 0.47 | 0.28 | 0.48 | 0.71 | 0.42 | 0.60 | 1.69 |
| ERCC1; PMK2; PARP1 | 9.64E−02 | 0.67 | 0.69 | 0.23 | 0.47 | 0.78 | 0.45 | 0.71 | 2.11 |
| RAD51; PMK2; ATM | 1.04E−01 | 0.64 | 0.70 | 0.20 | 0.45 | 0.79 | 0.48 | 0.73 | 2.09 |
| ERCC1; PMK2; ATM | 1.06E−01 | 0.63 | 0.62 | 0.25 | 0.40 | 0.81 | 0.49 | 0.75 | 2.13 |
| ERCC1; RAD51; PMK2 | 1.16E−01 | 0.65 | 0.53 | 0.21 | 0.44 | 0.76 | 0.46 | 0.60 | 1.85 |
| NQ01; RAD51; PMK2 | 1.20E−01 | 0.65 | 0.73 | 0.27 | 0.39 | 0.76 | 0.51 | 0.88 | 1.65 |
| ERCC1; PAR; PARP1 | 1.22E−01 | 0.63 | 0.17 | 0.21 | 0.45 | 0.79 | 0.36 | 0.30 | 2.12 |
| BRCA1; MLH1; PAR | 1.22E−01 | 0.66 | 0.27 | 0.18 | 0.47 | 0.75 | 0.41 | 0.36 | 1.88 |
| BRCA1; PARP1; ATM | 1.26E−01 | 0.63 | 0.16 | 0.15 | 0.50 | 0.80 | 0.35 | 0.23 | 2.50 |
| MLH1; PAR; ATM | 1.37E−01 | 0.62 | 0.34 | 0.24 | 0.48 | 0.73 | 0.42 | 0.48 | 1.79 |
| PAR; PMK2; ATM | 1.54E−01 | 0.60 | 0.57 | 0.19 | 0.43 | 0.80 | 0.49 | 0.66 | 2.16 |
| NQ01; PAR; PMK2 | 1.80E−01 | 0.58 | 0.66 | 0.25 | 0.40 | 0.75 | 0.52 | 0.83 | 1.58 |
| RAD51; PMK2; PARP1 | 1.80E−01 | 0.63 | 0.36 | 0.15 | 0.44 | 0.75 | 0.46 | 0.41 | 1.78 |
| NQ01; RAD51; ATM | 1.95E−01 | 0.59 | 0.53 | 0.25 | 0.38 | 0.74 | 0.51 | 0.72 | 1.45 |
| RAD51; BRCA1; MLH1 | 2.13E−01 | 0.68 | 0.19 | 0.20 | 0.43 | 0.80 | 0.38 | 0.31 | 2.14 |
| NQ01; PAR; ATM | 2.26E−01 | 0.56 | 0.41 | 0.27 | 0.43 | 0.71 | 0.47 | 0.61 | 1.46 |
| ERCC1; PAR; ATM | 2.95E−01 | 0.57 | 0.25 | 0.24 | 0.44 | 0.73 | 0.42 | 0.42 | 1.64 |
| ERCC1; RAD51; BRCA1 | 2.98E−01 | 0.65 | 0.16 | 0.17 | 0.50 | 0.73 | 0.36 | 0.26 | 1.88 |
| ERCC1; RAD51; PARP1 | 4.55E−01 | 0.65 | 0.03 | 0.17 | 0.50 | 0.79 | 0.25 | 0.16 | 2.33 |
| ERCC1; RAD51; ATM | | 0.61 | | 0.23 | | 0.76 | | 0.20 | |
| RAD51; BRCA1; PARP1 | | 0.60 | 0.09 | | 0.60 | | | 0.05 | |
| RAD51; PARP1; ATM | | 0.60 | | 0.12 | | 0.78 | | 0.10 | |
| PAR; PARP1; ATM | | 0.55 | | 0.15 | | 0.78 | | 0.12 | |

TABLE 11
Four Marker Probability Analysis TNBCMARKERS

| Markers | p val | AUC | Sens | Spec | PosPow | NegPow | AER | Frac.called | RelRisk |
|---|---|---|---|---|---|---|---|---|---|
| ERCC1; XPF; MLH1; PMK2 | 2.21E−06 | 0.77 | 0.52 | 0.42 | 0.73 | 0.86 | 0.20 | 0.57 | 5.27 |
| BRCA1; XPF; FANCD2; PMK2 | 4.13E−06 | 0.80 | 0.56 | 0.46 | 0.72 | 0.86 | 0.21 | 0.63 | 5.04 |
| XPF; MLH1; PMK2; PARP1 | 8.75E−06 | 0.77 | 0.42 | 0.42 | 0.70 | 0.86 | 0.20 | 0.53 | 5.08 |
| NQO1; BRCA1; XPF; FANCD2 | 1.01E−05 | 0.76 | 0.61 | 0.49 | 0.66 | 0.87 | 0.23 | 0.70 | 5.08 |
| NQO1; RAD51; XPF; FANCD2 | 1.45E−05 | 0.75 | 0.65 | 0.48 | 0.65 | 0.87 | 0.24 | 0.71 | 5.00 |
| NQO1; BRCA1; MLH1; FANCD2 | 1.78E−05 | 0.72 | 0.74 | 0.51 | 0.58 | 0.90 | 0.28 | 0.83 | 5.94 |
| RAD51; XPF; FANCD2; PMK2 | 2.96E−05 | 0.78 | 0.63 | 0.43 | 0.69 | 0.83 | 0.24 | 0.66 | 4.00 |
| RAD51; XPF; MLH1; PMK2 | 3.30E−05 | 0.78 | 0.42 | 0.41 | 0.70 | 0.83 | 0.22 | 0.53 | 4.20 |
| NQO1; XPF; PMK2; PARP1 | 3.89E−05 | 0.74 | 0.53 | 0.32 | 0.71 | 0.86 | 0.22 | 0.51 | 5.19 |
| NQO1; XPF; FANCD2; PARP1 | 4.01E−05 | 0.75 | 0.65 | 0.47 | 0.63 | 0.87 | 0.26 | 0.72 | 4.69 |
| ERCC1; NQO1; RAD51; XPF | 4.45E−05 | 0.74 | 0.45 | 0.35 | 0.64 | 0.92 | 0.22 | 0.49 | 7.64 |
| NQO1; XPF; MLH1; PMK2 | 4.80E−05 | 0.77 | 0.63 | 0.36 | 0.63 | 0.91 | 0.25 | 0.60 | 7.19 |
| NQO1; MLH1; FANCD2; PARP1 | 5.13E−05 | 0.70 | 0.71 | 0.49 | 0.55 | 0.90 | 0.30 | 0.81 | 5.50 |
| NQO1; RAD51; MLH1; FANCD2 | 5.45E−05 | 0.71 | 0.71 | 0.50 | 0.54 | 0.90 | 0.31 | 0.83 | 5.54 |
| XPF; PAR; FANCD2; PMK2 | 5.51E−05 | 0.77 | 0.54 | 0.49 | 0.68 | 0.85 | 0.23 | 0.66 | 4.43 |
| NQO1; BRCA1; XPF; PMK2 | 6.09E−05 | 0.79 | 0.50 | 0.34 | 0.67 | 0.87 | 0.23 | 0.52 | 5.11 |
| XPF; FANCD2; PMK2; PARP1 | 6.40E−05 | 0.78 | 0.64 | 0.43 | 0.66 | 0.85 | 0.25 | 0.68 | 4.43 |
| ERCC1; NQO1; BRCA1; XPF | 6.56E−05 | 0.77 | 0.47 | 0.38 | 0.65 | 0.85 | 0.24 | 0.54 | 4.40 |
| NQO1; XPF; MLH1; FANCD2 | 6.88E−05 | 0.76 | 0.68 | 0.52 | 0.60 | 0.85 | 0.28 | 0.79 | 4.08 |
| ERCC1; BRCA1; XPF; MLH1 | 7.48E−05 | 0.73 | 0.41 | 0.60 | 0.59 | 0.84 | 0.25 | 0.71 | 3.63 |
| RAD51; XPF; MLH1; PAR | 7.62E−05 | 0.70 | 0.41 | 0.54 | 0.71 | 0.80 | 0.23 | 0.64 | 3.53 |
| BRCA1; XPF; MLH1; PMK2 | 7.85E−05 | 0.78 | 0.41 | 0.43 | 0.62 | 0.89 | 0.22 | 0.54 | 5.78 |
| RAD51; MLH1; FANCD2; PMK2 | 8.13E−05 | 0.74 | 0.72 | 0.47 | 0.55 | 0.90 | 0.31 | 0.82 | 5.29 |
| BRCA1; MLH1; FANCD2; PMK2 | 9.80E−05 | 0.75 | 0.68 | 0.50 | 0.54 | 0.90 | 0.31 | 0.82 | 5.21 |
| ERCC1; NQO1; XPF; PARP1 | 1.01E−04 | 0.74 | 0.47 | 0.36 | 0.68 | 0.85 | 0.23 | 0.52 | 4.43 |
| ERCC1; NQO1; XPF; FANCD2 | 1.02E−04 | 0.78 | 0.63 | 0.51 | 0.54 | 0.88 | 0.30 | 0.79 | 4.34 |
| ERCC1; NQO1; MLH1; FANCD2 | 1.05E−04 | 0.71 | 0.73 | 0.49 | 0.56 | 0.84 | 0.31 | 0.84 | 3.61 |
| NQO1; XPF; FANCD2; PMK2 | 1.08E−04 | 0.78 | 0.68 | 0.51 | 0.55 | 0.87 | 0.30 | 0.82 | 4.28 |
| ERCC1; NQO1; XPF; MLH1 | 1.17E−04 | 0.76 | 0.50 | 0.46 | 0.55 | 0.85 | 0.29 | 0.67 | 3.64 |
| NQO1; RAD51; XPF; PMK2 | 1.17E−04 | 0.76 | 0.47 | 0.30 | 0.68 | 0.86 | 0.23 | 0.47 | 4.77 |
| XPF; MLH1; FANCD2; PMK2 | 1.17E−04 | 0.79 | 0.72 | 0.49 | 0.61 | 0.84 | 0.29 | 0.80 | 3.87 |
| ERCC1; NQO1; XPF; ATM | 1.19E−04 | 0.71 | 0.40 | 0.46 | 0.63 | 0.86 | 0.23 | 0.57 | 4.58 |
| NQO1; XPF; MLH1; PARP1 | 1.25E−04 | 0.71 | 0.45 | 0.41 | 0.63 | 0.83 | 0.26 | 0.57 | 3.75 |
| BRCA1; XPF; MLH1; PAR | 1.25E−04 | 0.70 | 0.43 | 0.62 | 0.62 | 0.82 | 0.25 | 0.74 | 3.36 |
| BRCA1; FANCD2; PMK2; PARP1 | 1.51E−04 | 0.73 | 0.69 | 0.45 | 0.56 | 0.88 | 0.31 | 0.78 | 4.89 |
| RAD51; BRCA1; XPF; PMK2 | 1.55E−04 | 0.77 | 0.38 | 0.24 | 0.71 | 0.93 | 0.19 | 0.35 | 10.59 |
| ERCC1; RAD51; XPF; PMK2 | 1.59E−04 | 0.75 | 0.45 | 0.33 | 0.70 | 0.83 | 0.23 | 0.48 | 4.20 |
| ERCC1; BRCA1; XPF; PMK2 | 1.63E−04 | 0.78 | 0.44 | 0.34 | 0.70 | 0.83 | 0.23 | 0.49 | 4.20 |
| RAD51; BRCA1; XPF; MLH1 | 1.73E−04 | 0.71 | 0.41 | 0.57 | 0.62 | 0.81 | 0.25 | 0.69 | 3.33 |
| ERCC1; XPF; MLH1; PARP1 | 1.92E−04 | 0.72 | 0.47 | 0.46 | 0.65 | 0.80 | 0.26 | 0.62 | 3.26 |
| ERCC1; BRCA1; MLH1; FANCD2 | 1.94E−04 | 0.71 | 0.73 | 0.46 | 0.55 | 0.89 | 0.31 | 0.81 | 5.13 |
| MLH1; FANCD2; PMK2; PARP1 | 2.09E−04 | 0.73 | 0.66 | 0.45 | 0.54 | 0.89 | 0.32 | 0.78 | 4.85 |
| RAD51; PAR; PARP1; ATM | 2.14E−04 | 0.58 | 0.07 | 0.17 | 1.00 | 0.80 | 0.17 | 0.16 | 5.00 |
| ERCC1; XPF; PMK2; PARP1 | 2.17E−04 | 0.75 | 0.47 | 0.32 | 0.71 | 0.83 | 0.23 | 0.48 | 4.11 |
| NQO1; MLH1; PAR; FANCD2 | 2.17E−04 | 0.69 | 0.71 | 0.49 | 0.56 | 0.88 | 0.31 | 0.83 | 4.81 |
| RAD51; BRCA1; FANCD2; PMK2 | 2.22E−04 | 0.73 | 0.69 | 0.48 | 0.55 | 0.86 | 0.32 | 0.82 | 3.99 |
| ERCC1; XPF; PAR; PMK2 | 2.23E−04 | 0.74 | 0.43 | 0.35 | 0.71 | 0.85 | 0.22 | 0.48 | 4.71 |
| BRCA1; XPF; MLH1; FANCD2 | 2.26E−04 | 0.73 | 0.58 | 0.51 | 0.58 | 0.85 | 0.28 | 0.74 | 3.83 |
| ERCC1; BRCA1; XPF; FANCD2 | 2.28E−04 | 0.74 | 0.58 | 0.51 | 0.58 | 0.85 | 0.28 | 0.74 | 3.83 |
| ERCC1; NQO1; BRCA1; FANCD2 | 2.32E−04 | 0.72 | 0.74 | 0.48 | 0.55 | 0.84 | 0.33 | 0.86 | 3.40 |
| ERCC1; NQO1; RAD51; FANCD2 | 2.43E−04 | 0.72 | 0.74 | 0.47 | 0.56 | 0.81 | 0.33 | 0.85 | 2.99 |
| XPF; PAR; PMK2; ATM | 2.46E−04 | 0.71 | 0.43 | 0.35 | 0.75 | 0.88 | 0.18 | 0.46 | 6.38 |
| XPF; MLH1; PAR; PMK2 | 2.71E−04 | 0.75 | 0.45 | 0.33 | 0.72 | 0.80 | 0.24 | 0.49 | 3.61 |
| NQO1; XPF; PAR; PMK2 | 2.90E−04 | 0.71 | 0.52 | 0.35 | 0.65 | 0.89 | 0.24 | 0.55 | 6.20 |
| ERCC1; RAD51; XPF; PAR | 3.02E−04 | 0.71 | 0.36 | 0.33 | 0.71 | 0.85 | 0.21 | 0.43 | 4.76 |
| ERCC1; XPF; MLH1; FANCD2 | 3.04E−04 | 0.75 | 0.60 | 0.55 | 0.53 | 0.86 | 0.30 | 0.81 | 3.81 |
| ERCC1; XPF; FANCD2; PMK2 | 3.06E−04 | 0.78 | 0.68 | 0.46 | 0.55 | 0.86 | 0.31 | 0.79 | 4.01 |
| MLH1; FANCD2; PMK2; ATM | 3.08E−04 | 0.74 | 0.76 | 0.46 | 0.58 | 0.88 | 0.30 | 0.82 | 4.82 |
| NQO1; BRCA1; FANCD2; PARP1 | 3.16E−04 | 0.71 | 0.68 | 0.48 | 0.53 | 0.87 | 0.33 | 0.82 | 3.94 |
| NQO1; RAD51; BRCA1; FANCD2 | 3.19E−04 | 0.72 | 0.63 | 0.49 | 0.51 | 0.87 | 0.33 | 0.80 | 3.97 |
| RAD51; XPF; MLH1; FANCD2 | 3.20E−04 | 0.74 | 0.66 | 0.50 | 0.55 | 0.85 | 0.31 | 0.80 | 3.76 |
| XPF; MLH1; PMK2; ATM | 3.23E−04 | 0.75 | 0.50 | 0.36 | 0.71 | 0.83 | 0.23 | 0.53 | 4.11 |
| ERCC1; NQO1; XPF; PMK2 | 3.24E−04 | 0.78 | 0.77 | 0.41 | 0.51 | 0.89 | 0.35 | 0.82 | 4.60 |
| ERCC1; RAD51; XPF; MLH1 | 3.30E−04 | 0.73 | 0.42 | 0.47 | 0.62 | 0.83 | 0.25 | 0.60 | 3.61 |
| ERCC1; NQO1; XPF; PAR | 3.41E−04 | 0.73 | 0.48 | 0.35 | 0.67 | 0.86 | 0.24 | 0.53 | 4.67 |
| NQO1; XPF; PAR; FANCD2 | 3.44E−04 | 0.75 | 0.57 | 0.49 | 0.59 | 0.85 | 0.28 | 0.72 | 4.00 |
| ERCC1; BRCA1; XPF; PARP1 | 3.70E−04 | 0.73 | 0.39 | 0.44 | 0.59 | 0.84 | 0.26 | 0.57 | 3.78 |
| NQO1; RAD51; XPF; ATM | 3.90E−04 | 0.68 | 0.40 | 0.36 | 0.57 | 0.91 | 0.26 | 0.51 | 6.29 |
| NQO1; XPF; MLH1; ATM | 3.90E−04 | 0.70 | 0.42 | 0.50 | 0.57 | 0.84 | 0.27 | 0.65 | 3.62 |
| NQO1; BRCA1; XPF; MLH1 | 3.99E−04 | 0.74 | 0.42 | 0.46 | 0.58 | 0.82 | 0.28 | 0.62 | 3.31 |
| NQO1; MLH1; FANCD2; ATM | 4.02E−04 | 0.68 | 0.69 | 0.49 | 0.54 | 0.89 | 0.31 | 0.82 | 4.86 |
| NQO1; XPF; FANCD2; ATM | 4.24E−04 | 0.73 | 0.55 | 0.51 | 0.53 | 0.89 | 0.29 | 0.74 | 4.98 |
| NQO1; RAD51; XPF; MLH1 | 4.29E−04 | 0.74 | 0.47 | 0.37 | 0.63 | 0.82 | 0.27 | 0.55 | 3.50 |
| ERCC1; XPF; PAR; PARP1 | 4.37E−04 | 0.69 | 0.41 | 0.33 | 0.71 | 0.81 | 0.24 | 0.47 | 3.71 |
| MLH1; PAR; FANCD2; PMK2 | 4.66E−04 | 0.72 | 0.71 | 0.47 | 0.56 | 0.88 | 0.32 | 0.82 | 4.44 |
| NQO1; MLH1; FANCD2; PMK2 | 4.85E−04 | 0.73 | 0.71 | 0.49 | 0.51 | 0.87 | 0.34 | 0.87 | 3.84 |
| ERCC1; RAD51; MLH1; FANCD2 | 4.87E−04 | 0.71 | 0.77 | 0.46 | 0.53 | 0.87 | 0.33 | 0.85 | 4.01 |
| XPF; FANCD2; PMK2; ATM | 5.45E−04 | 0.75 | 0.66 | 0.44 | 0.58 | 0.88 | 0.30 | 0.74 | 4.61 |
| RAD51; FANCD2; PMK2; PARP1 | 5.89E−04 | 0.73 | 0.69 | 0.41 | 0.52 | 0.88 | 0.34 | 0.78 | 4.37 |
| ERCC1; NQO1; BRCA1; MLH1 | 6.01E−04 | 0.70 | 0.59 | 0.35 | 0.53 | 0.88 | 0.33 | 0.65 | 4.22 |
| XPF; MLH1; FANCD2; PARP1 | 6.06E−04 | 0.73 | 0.63 | 0.46 | 0.57 | 0.84 | 0.30 | 0.75 | 3.54 |
| RAD51; XPF; PAR; FANCD2 | 6.31E−04 | 0.74 | 0.46 | 0.50 | 0.59 | 0.86 | 0.26 | 0.66 | 4.14 |
| ERCC1; XPF; FANCD2; PARP1 | 6.64E−04 | 0.73 | 0.58 | 0.42 | 0.56 | 0.86 | 0.30 | 0.68 | 3.94 |
| RAD51; BRCA1; XPF; PAR | 6.77E−04 | 0.68 | 0.34 | 0.52 | 0.67 | 0.76 | 0.27 | 0.62 | 2.83 |
| NQO1; BRCA1; FANCD2; PMK2 | 6.89E−04 | 0.73 | 0.69 | 0.46 | 0.55 | 0.80 | 0.34 | 0.83 | 2.75 |
| RAD51; XPF; MLH1; PARP1 | 7.14E−04 | 0.71 | 0.39 | 0.45 | 0.59 | 0.82 | 0.27 | 0.59 | 3.35 |
| ERCC1; NQO1; FANCD2; PMK2 | 7.64E−04 | 0.70 | 0.77 | 0.46 | 0.55 | 0.80 | 0.35 | 0.89 | 2.73 |
| ERCC1; XPF; PAR; FANCD2 | 7.80E−04 | 0.74 | 0.52 | 0.50 | 0.56 | 0.86 | 0.28 | 0.71 | 3.92 |
| ERCC1; RAD51; XPF; FANCD2 | 8.98E−04 | 0.73 | 0.50 | 0.50 | 0.52 | 0.88 | 0.29 | 0.70 | 4.27 |
| BRCA1; MLH1; PAR; PMK2 | 9.26E−04 | 0.69 | 0.55 | 0.28 | 0.62 | 0.93 | 0.28 | 0.53 | 8.62 |
| ERCC1; MLH1; PAR; FANCD2 | 9.45E−04 | 0.69 | 0.74 | 0.46 | 0.49 | 0.92 | 0.35 | 0.87 | 5.85 |
| RAD51; MLH1; PAR; FANCD2 | 9.50E−04 | 0.74 | 0.75 | 0.42 | 0.58 | 0.87 | 0.31 | 0.78 | 4.47 |
| BRCA1; MLH1; PAR; FANCD2 | 9.86E−04 | 0.71 | 0.71 | 0.48 | 0.57 | 0.85 | 0.31 | 0.82 | 3.71 |
| ERCC1; NQO1; FANCD2; PARP1 | 9.86E−04 | 0.70 | 0.77 | 0.43 | 0.52 | 0.82 | 0.36 | 0.86 | 2.93 |
| NQO1; XPF; MLH1; PAR | 9.91E−04 | 0.69 | 0.43 | 0.39 | 0.59 | 0.80 | 0.30 | 0.58 | 2.95 |
| BRCA1; XPF; PMK2; PARP1 | 1.02E−03 | 0.77 | 0.39 | 0.22 | 0.68 | 0.87 | 0.24 | 0.37 | 5.13 |
| BRCA1; XPF; PAR; PMK2 | 1.03E−03 | 0.74 | 0.38 | 0.30 | 0.69 | 0.82 | 0.24 | 0.43 | 3.90 |
| XPF; PAR; PMK2; PARP1 | 1.03E−03 | 0.71 | 0.38 | 0.29 | 0.69 | 0.82 | 0.24 | 0.42 | 3.90 |
| NQO1; RAD51; FANCD2; PARP1 | 1.04E−03 | 0.71 | 0.68 | 0.45 | 0.51 | 0.83 | 0.35 | 0.83 | 3.07 |
| BRCA1; XPF; PAR; FANCD2 | 1.04E−03 | 0.75 | 0.46 | 0.48 | 0.59 | 0.85 | 0.27 | 0.65 | 3.84 |
| ERCC1; MLH1; FANCD2; PMK2 | 1.05E−03 | 0.73 | 0.70 | 0.45 | 0.54 | 0.83 | 0.34 | 0.82 | 3.12 |
| NQO1; RAD51; XPF; PARP1 | 1.06E−03 | 0.69 | 0.38 | 0.24 | 0.60 | 0.88 | 0.27 | 0.39 | 5.10 |
| ERCC1; XPF; MLH1; ATM | 1.07E−03 | 0.68 | 0.40 | 0.44 | 0.63 | 0.80 | 0.27 | 0.58 | 3.16 |
| ERCC1; MLH1; FANCD2; PARP1 | 1.22E−03 | 0.69 | 0.70 | 0.42 | 0.50 | 0.88 | 0.35 | 0.80 | 4.33 |
| RAD51; BRCA1; MLH1; PMK2 | 1.26E−03 | 0.71 | 0.50 | 0.26 | 0.55 | 0.94 | 0.31 | 0.50 | 8.83 |
| BRCA1; XPF; PMK2; ATM | 1.28E−03 | 0.74 | 0.37 | 0.29 | 0.65 | 0.88 | 0.24 | 0.42 | 5.50 |
| ERCC1; NQO1; PAR; FANCD2 | 1.28E−03 | 0.69 | 0.78 | 0.40 | 0.54 | 0.83 | 0.35 | 0.84 | 3.10 |
| RAD51; MLH1; FANCD2; PARP1 | 1.31E−03 | 0.71 | 0.75 | 0.39 | 0.57 | 0.85 | 0.32 | 0.77 | 3.71 |
| NQO1; BRCA1; XPF; ATM | 1.32E−03 | 0.68 | 0.35 | 0.41 | 0.58 | 0.85 | 0.27 | 0.53 | 3.76 |
| ERCC1; NQO1; MLH1; PMK2 | 1.34E−03 | 0.70 | 0.84 | 0.36 | 0.49 | 0.88 | 0.39 | 0.87 | 3.92 |
| XPF; PAR; FANCD2; PARP1 | 1.36E−03 | 0.73 | 0.50 | 0.48 | 0.56 | 0.85 | 0.29 | 0.68 | 3.78 |
| NQO1; BRCA1; PAR; FANCD2 | 1.38E−03 | 0.70 | 0.61 | 0.46 | 0.52 | 0.88 | 0.33 | 0.77 | 4.12 |
| NQO1; BRCA1; FANCD2; ATM | 1.41E−03 | 0.71 | 0.62 | 0.46 | 0.51 | 0.88 | 0.33 | 0.78 | 4.29 |
| RAD51; BRCA1; PAR; FANCD2 | 1.43E−03 | 0.74 | 0.54 | 0.35 | 0.63 | 0.84 | 0.28 | 0.58 | 3.96 |
| ERCC1; BRCA1; MLH1; PARP1 | 1.45E−03 | 0.69 | 0.41 | 0.36 | 0.57 | 0.84 | 0.29 | 0.53 | 3.53 |
| ERCC1; NQO1; RAD51; MLH1 | 1.53E−03 | 0.70 | 0.68 | 0.34 | 0.48 | 0.88 | 0.38 | 0.74 | 3.82 |
| BRCA1; PAR; FANCD2; PMK2 | 1.55E−03 | 0.73 | 0.64 | 0.42 | 0.53 | 0.86 | 0.35 | 0.77 | 3.71 |
| XPF; MLH1; PAR; FANCD2 | 1.61E−03 | 0.74 | 0.54 | 0.50 | 0.54 | 0.86 | 0.30 | 0.74 | 3.75 |
| RAD51; XPF; PAR; PMK2 | 1.62E−03 | 0.72 | 0.38 | 0.27 | 0.69 | 0.81 | 0.25 | 0.41 | 3.67 |
| ERCC1; RAD51; BRCA1; PAR | 1.64E−03 | 0.71 | 0.32 | 0.25 | 0.69 | 0.87 | 0.21 | 0.35 | 5.19 |
| NQO1; FANCD2; PARP1; ATM | 1.70E−03 | 0.68 | 0.69 | 0.45 | 0.53 | 0.85 | 0.34 | 0.82 | 3.42 |
| ERCC1; RAD51; XPF; PARP1 | 1.71E−03 | 0.69 | 0.35 | 0.36 | 0.61 | 0.79 | 0.28 | 0.49 | 2.95 |
| NQO1; RAD51; FANCD2; ATM | 1.74E−03 | 0.67 | 0.69 | 0.45 | 0.53 | 0.85 | 0.34 | 0.82 | 3.42 |
| ERCC1; RAD51; BRCA1; XPF | 1.82E−03 | 0.73 | 0.35 | 0.37 | 0.61 | 0.82 | 0.26 | 0.49 | 3.42 |
| PAR; FANCD2; PMK2; PARP1 | 1.83E−03 | 0.70 | 0.64 | 0.42 | 0.51 | 0.86 | 0.35 | 0.78 | 3.77 |
| ERCC1; BRCA1; MLH1; PMK2 | 1.90E−03 | 0.71 | 0.58 | 0.32 | 0.55 | 0.86 | 0.33 | 0.61 | 3.82 |
| ERCC1; XPF; PMK2; ATM | 1.94E−03 | 0.72 | 0.41 | 0.35 | 0.71 | 0.78 | 0.25 | 0.49 | 3.25 |
| BRCA1; XPF; MLH1; PARP1 | 1.94E−03 | 0.71 | 0.39 | 0.55 | 0.57 | 0.79 | 0.29 | 0.70 | 2.64 |
| RAD51; XPF; PMK2; PARP1 | 1.95E−03 | 0.73 | 0.39 | 0.18 | 0.65 | 0.92 | 0.25 | 0.34 | 7.80 |
| NQO1; RAD51; FANCD2; PMK2 | 2.02E−03 | 0.73 | 0.69 | 0.45 | 0.51 | 0.80 | 0.37 | 0.86 | 2.56 |
| NQO1; XPF; PARP1; ATM | 2.08E−03 | 0.67 | 0.39 | 0.35 | 0.57 | 0.83 | 0.30 | 0.51 | 3.29 |
| ERCC1; RAD51; BRCA1; MLH1 | 2.11E−03 | 0.69 | 0.39 | 0.27 | 0.60 | 0.84 | 0.28 | 0.43 | 3.80 |
| ERCC1; XPF; MLH1; PAR | 2.14E−03 | 0.70 | 0.41 | 0.42 | 0.63 | 0.76 | 0.29 | 0.59 | 2.62 |
| RAD51; XPF; PMK2; ATM | 2.16E−03 | 0.72 | 0.37 | 0.26 | 0.65 | 0.88 | 0.24 | 0.39 | 5.18 |
| BRCA1; FANCD2; PMK2; ATM | 2.24E−03 | 0.73 | 0.69 | 0.38 | 0.56 | 0.85 | 0.34 | 0.76 | 3.70 |
| ERCC1; NQO1; FANCD2; ATM | 2.26E−03 | 0.68 | 0.79 | 0.42 | 0.55 | 0.80 | 0.35 | 0.86 | 2.75 |
| ERCC1; RAD51; PAR; PARP1 | 2.27E−03 | 0.69 | 0.25 | 0.25 | 0.70 | 0.81 | 0.23 | 0.32 | 3.73 |
| RAD51; PAR; FANCD2; PARP1 | 2.29E−03 | 0.73 | 0.50 | 0.35 | 0.58 | 0.89 | 0.28 | 0.57 | 5.54 |
| RAD51; PAR; FANCD2; PMK2 | 2.32E−03 | 0.75 | 0.68 | 0.40 | 0.53 | 0.86 | 0.35 | 0.78 | 3.69 |
| RAD51; BRCA1; XPF; PARP1 | 2.35E−03 | 0.67 | 0.34 | 0.53 | 0.58 | 0.79 | 0.28 | 0.65 | 2.70 |
| NQO1; BRCA1; MLH1; PMK2 | 2.36E−03 | 0.70 | 0.72 | 0.34 | 0.49 | 0.87 | 0.39 | 0.78 | 3.75 |
| ERCC1; NQO1; PMK2; ATM | 2.46E−03 | 0.63 | 0.76 | 0.39 | 0.49 | 0.87 | 0.38 | 0.85 | 3.75 |
| RAD51; FANCD2; PMK2; ATM | 2.49E−03 | 0.71 | 0.69 | 0.40 | 0.53 | 0.86 | 0.35 | 0.78 | 3.86 |
| MLH1; PAR; FANCD2; PARP1 | 2.66E−03 | 0.69 | 0.71 | 0.44 | 0.51 | 0.88 | 0.35 | 0.83 | 4.10 |
| BRCA1; MLH1; PMK2; PARP1 | 2.66E−03 | 0.70 | 0.50 | 0.25 | 0.57 | 0.88 | 0.32 | 0.49 | 4.57 |
| RAD51; BRCA1; MLH1; FANCD2 | 2.67E−03 | 0.71 | 0.65 | 0.42 | 0.53 | 0.85 | 0.34 | 0.76 | 3.55 |
| NQO1; FANCD2; PMK2; PARP1 | 2.68E−03 | 0.70 | 0.71 | 0.44 | 0.49 | 0.82 | 0.38 | 0.88 | 2.74 |
| BRCA1; PAR; FANCD2; PARP1 | 2.73E−03 | 0.70 | 0.50 | 0.39 | 0.56 | 0.86 | 0.30 | 0.62 | 3.92 |
| ERCC1; BRCA1; FANCD2; PMK2 | 2.78E−03 | 0.72 | 0.63 | 0.45 | 0.53 | 0.79 | 0.36 | 0.81 | 2.54 |
| ERCC1; XPF; FANCD2; ATM | 2.84E−03 | 0.70 | 0.54 | 0.46 | 0.52 | 0.88 | 0.31 | 0.71 | 4.48 |
| NQO1; PAR; FANCD2; PARP1 | 2.90E−03 | 0.67 | 0.64 | 0.40 | 0.51 | 0.86 | 0.35 | 0.76 | 3.77 |
| NQO1; RAD51; BRCA1; XPF | 3.02E−03 | 0.74 | 0.38 | 0.26 | 0.60 | 0.84 | 0.28 | 0.41 | 3.80 |
| ERCC1; XPF; PARP1; ATM | 3.10E−03 | 0.66 | 0.37 | 0.38 | 0.61 | 0.81 | 0.27 | 0.51 | 3.18 |
| ERCC1; RAD51; BRCA1; FANCD2 | 3.16E−03 | 0.69 | 0.58 | 0.40 | 0.50 | 0.85 | 0.35 | 0.72 | 3.25 |
| ERCC1; BRCA1; FANCD2; PARP1 | 3.17E−03 | 0.69 | 0.58 | 0.39 | 0.53 | 0.84 | 0.34 | 0.69 | 3.31 |
| NQO1; XPF; PMK2; ATM | 3.20E−03 | 0.74 | 0.57 | 0.38 | 0.55 | 0.83 | 0.33 | 0.67 | 3.29 |
| XPF; PMK2; PARP1; ATM | 3.22E−03 | 0.73 | 0.37 | 0.25 | 0.65 | 0.87 | 0.25 | 0.39 | 4.85 |
| ERCC1; NQO1; MLH1; PARP1 | 3.31E−03 | 0.67 | 0.66 | 0.33 | 0.49 | 0.83 | 0.39 | 0.73 | 2.93 |
| ERCC1; NQO1; MLH1; ATM | 3.40E−03 | 0.66 | 0.60 | 0.45 | 0.47 | 0.83 | 0.37 | 0.81 | 2.75 |
| RAD51; XPF; MLH1; ATM | 3.48E−03 | 0.68 | 0.40 | 0.48 | 0.60 | 0.79 | 0.28 | 0.63 | 2.91 |
| XPF; MLH1; PARP1; ATM | 3.55E−03 | 0.67 | 0.39 | 0.45 | 0.57 | 0.81 | 0.29 | 0.60 | 2.95 |
| RAD51; BRCA1; PAR; PARP1 | 3.55E−03 | 0.62 | 0.24 | 0.14 | 0.78 | 0.78 | 0.22 | 0.23 | 3.50 |
| RAD51; BRCA1; PAR; ATM | 3.71E−03 | 0.60 | 0.25 | 0.18 | 0.78 | 0.80 | 0.21 | 0.26 | 3.89 |
| NQO1; PAR; FANCD2; PMK2 | 3.79E−03 | 0.67 | 0.71 | 0.39 | 0.51 | 0.85 | 0.37 | 0.82 | 3.42 |
| RAD51; FANCD2; PARP1; ATM | 3.91E−03 | 0.67 | 0.62 | 0.35 | 0.50 | 0.90 | 0.36 | 0.70 | 5.00 |
| ERCC1; BRCA1; XPF; PAR | 3.95E−03 | 0.72 | 0.38 | 0.34 | 0.61 | 0.77 | 0.30 | 0.51 | 2.69 |
| ERCC1; NQO1; BRCA1; PARP1 | 4.02E−03 | 0.67 | 0.63 | 0.31 | 0.48 | 0.83 | 0.40 | 0.70 | 2.74 |
| NQO1; RAD51; MLH1; ATM | 4.02E−03 | 0.65 | 0.60 | 0.39 | 0.47 | 0.84 | 0.38 | 0.75 | 2.96 |
| FANCD2; PMK2; PARP1; ATM | 4.22E−03 | 0.71 | 0.66 | 0.40 | 0.50 | 0.86 | 0.37 | 0.79 | 3.67 |
| XPF; MLH1; PAR; PARP1 | 4.24E−03 | 0.69 | 0.40 | 0.46 | 0.57 | 0.77 | 0.31 | 0.63 | 2.53 |
| ERCC1; RAD51; BRCA1; PMK2 | 4.48E−03 | 0.68 | 0.47 | 0.26 | 0.60 | 0.79 | 0.32 | 0.49 | 2.85 |
| NQO1; XPF; PAR; ATM | 4.55E−03 | 0.65 | 0.38 | 0.38 | 0.58 | 0.81 | 0.30 | 0.54 | 3.04 |
| NQO1; MLH1; PMK2; ATM | 4.64E−03 | 0.69 | 0.67 | 0.39 | 0.50 | 0.83 | 0.38 | 0.79 | 3.00 |
| ERCC1; BRCA1; PAR; FANCD2 | 4.88E−03 | 0.70 | 0.63 | 0.41 | 0.53 | 0.83 | 0.35 | 0.75 | 3.05 |
| XPF; PAR; FANCD2; ATM | 4.94E−03 | 0.71 | 0.52 | 0.48 | 0.58 | 0.83 | 0.29 | 0.70 | 3.50 |
| RAD51; XPF; PARP1; ATM | 5.27E−03 | 0.64 | 0.33 | 0.32 | 0.63 | 0.78 | 0.28 | 0.45 | 2.88 |
| ERCC1; RAD51; FANCD2; PARP1 | 5.34E−03 | 0.69 | 0.70 | 0.37 | 0.50 | 0.84 | 0.37 | 0.77 | 3.13 |
| NQO1; FANCD2; PMK2; ATM | 5.40E−03 | 0.70 | 0.72 | 0.41 | 0.53 | 0.79 | 0.38 | 0.85 | 2.52 |
| NQO1; RAD51; MLH1; PARP1 | 5.42E−03 | 0.67 | 0.53 | 0.25 | 0.53 | 0.83 | 0.36 | 0.54 | 3.19 |
| NQO1; RAD51; BRCA1; PARP1 | 5.48E−03 | 0.65 | 0.31 | 0.23 | 0.56 | 0.82 | 0.31 | 0.37 | 3.15 |
| RAD51; MLH1; FANCD2; ATM | 5.52E−03 | 0.70 | 0.69 | 0.41 | 0.49 | 0.88 | 0.37 | 0.81 | 3.90 |
| NQO1; PAR; FANCD2; ATM | 5.59E−03 | 0.66 | 0.63 | 0.44 | 0.52 | 0.86 | 0.35 | 0.79 | 3.61 |
| NQO1; BRCA1; PAR; PARP1 | 5.65E−03 | 0.60 | 0.33 | 0.25 | 0.56 | 0.81 | 0.32 | 0.42 | 2.96 |
| BRCA1; FANCD2; PARP1; ATM | 5.87E−03 | 0.68 | 0.52 | 0.37 | 0.54 | 0.86 | 0.33 | 0.63 | 3.75 |
| RAD51; BRCA1; FANCD2; PARP1 | 5.91E−03 | 0.69 | 0.52 | 0.33 | 0.53 | 0.86 | 0.33 | 0.59 | 3.73 |
| NQO1; RAD51; PAR; FANCD2 | 5.94E−03 | 0.72 | 0.61 | 0.40 | 0.50 | 0.83 | 0.37 | 0.76 | 2.88 |
| NQO1; BRCA1; MLH1; ATM | 5.95E−03 | 0.67 | 0.52 | 0.38 | 0.53 | 0.80 | 0.35 | 0.65 | 2.67 |
| BRCA1; MLH1; PARP1; ATM | 5.98E−03 | 0.67 | 0.39 | 0.36 | 0.55 | 0.83 | 0.31 | 0.54 | 3.14 |
| NQO1; RAD51; BRCA1; MLH1 | 6.05E−03 | 0.69 | 0.47 | 0.25 | 0.50 | 0.88 | 0.36 | 0.51 | 4.25 |
| ERCC1; MLH1; FANCD2; ATM | 6.07E−03 | 0.67 | 0.68 | 0.43 | 0.48 | 0.88 | 0.38 | 0.83 | 3.80 |
| RAD51; MLH1; PMK2; PARP1 | 6.11E−03 | 0.69 | 0.58 | 0.20 | 0.58 | 0.86 | 0.34 | 0.51 | 4.03 |
| BRCA1; MLH1; FANCD2; PARP1 | 6.14E−03 | 0.70 | 0.68 | 0.39 | 0.55 | 0.81 | 0.34 | 0.75 | 2.87 |
| ERCC1; FANCD2; PMK2; PARP1 | 6.16E−03 | 0.72 | 0.65 | 0.40 | 0.50 | 0.81 | 0.38 | 0.79 | 2.60 |
| NQO1; RAD51; BRCA1; PAR | 6.73E−03 | 0.63 | 0.41 | 0.25 | 0.52 | 0.87 | 0.34 | 0.48 | 3.91 |
| MLH1; FANCD2; PARP1; ATM | 6.96E−03 | 0.69 | 0.72 | 0.40 | 0.49 | 0.87 | 0.38 | 0.84 | 3.74 |
| NQO1; BRCA1; XPF; PARP1 | 6.96E−03 | 0.71 | 0.36 | 0.25 | 0.60 | 0.79 | 0.31 | 0.41 | 2.85 |
| BRCA1; MLH1; PMK2; ATM | 6.96E−03 | 0.71 | 0.57 | 0.32 | 0.53 | 0.84 | 0.35 | 0.64 | 3.36 |
| ERCC1; NQO1; RAD51; BRCA1 | 7.06E−03 | 0.69 | 0.63 | 0.29 | 0.48 | 0.82 | 0.41 | 0.68 | 2.62 |
| NQO1; BRCA1; MLH1; PAR | 7.89E−03 | 0.64 | 0.47 | 0.26 | 0.56 | 0.81 | 0.34 | 0.51 | 2.99 |
| MLH1; PAR; FANCD2; ATM | 8.20E−03 | 0.67 | 0.74 | 0.45 | 0.50 | 0.86 | 0.37 | 0.90 | 3.67 |
| ERCC1; NQO1; RAD51; ATM | 8.27E−03 | 0.64 | 0.66 | 0.33 | 0.43 | 0.86 | 0.43 | 0.78 | 3.02 |
| RAD51; BRCA1; MLH1; PARP1 | 8.30E−03 | 0.68 | 0.25 | 0.28 | 0.57 | 0.81 | 0.29 | 0.38 | 3.00 |
| BRCA1; MLH1; FANCD2; ATM | 8.30E−03 | 0.70 | 0.69 | 0.42 | 0.53 | 0.83 | 0.35 | 0.81 | 3.16 |
| RAD51; MLH1; PAR; PARP1 | 8.35E−03 | 0.68 | 0.24 | 0.17 | 0.70 | 0.75 | 0.27 | 0.27 | 2.80 |
| ERCC1; NQO1; MLH1; PAR | 8.50E−03 | 0.64 | 0.59 | 0.33 | 0.46 | 0.85 | 0.40 | 0.71 | 3.06 |
| ERCC1; RAD51; FANCD2; PMK2 | 8.52E−03 | 0.71 | 0.61 | 0.43 | 0.49 | 0.79 | 0.38 | 0.80 | 2.35 |
| NQO1; MLH1; PMK2; PARP1 | 8.57E−03 | 0.67 | 0.66 | 0.33 | 0.45 | 0.86 | 0.42 | 0.77 | 3.28 |
| RAD51; MLH1; PARP1; ATM | 9.15E−03 | 0.66 | 0.40 | 0.31 | 0.57 | 0.81 | 0.31 | 0.49 | 3.00 |
| NQO1; BRCA1; MLH1; PARP1 | 9.25E−03 | 0.68 | 0.42 | 0.25 | 0.54 | 0.79 | 0.36 | 0.48 | 2.56 |
| XPF; MLH1; FANCD2; ATM | 9.33E−03 | 0.71 | 0.59 | 0.49 | 0.49 | 0.83 | 0.35 | 0.81 | 2.91 |
| ERCC1; BRCA1; FANCD2; ATM | 9.49E−03 | 0.66 | 0.57 | 0.42 | 0.50 | 0.83 | 0.36 | 0.74 | 3.00 |
| PAR; FANCD2; PARP1; ATM | 9.62E−03 | 0.66 | 0.56 | 0.36 | 0.52 | 0.88 | 0.35 | 0.67 | 4.40 |
| ERCC1; NQO1; BRCA1; ATM | 9.63E−03 | 0.67 | 0.60 | 0.38 | 0.45 | 0.83 | 0.41 | 0.77 | 2.70 |
| PAR; FANCD2; PMK2; ATM | 1.00E−02 | 0.71 | 0.67 | 0.38 | 0.53 | 0.83 | 0.37 | 0.79 | 3.18 |
| BRCA1; PAR; PMK2; PARP1 | 1.02E−02 | 0.65 | 0.55 | 0.26 | 0.55 | 0.80 | 0.36 | 0.58 | 2.76 |
| NQO1; RAD51; BRCA1; ATM | 1.03E−02 | 0.65 | 0.40 | 0.30 | 0.55 | 0.80 | 0.33 | 0.50 | 2.73 |
| ERCC1; NQO1; PMK2; PARP1 | 1.03E−02 | 0.64 | 0.74 | 0.34 | 0.41 | 0.87 | 0.46 | 0.89 | 3.15 |
| NQO1; BRCA1; PMK2; ATM | 1.04E−02 | 0.67 | 0.73 | 0.33 | 0.48 | 0.85 | 0.41 | 0.81 | 3.19 |
| RAD51; MLH1; PMK2; ATM | 1.07E−02 | 0.70 | 0.57 | 0.26 | 0.50 | 0.88 | 0.38 | 0.60 | 4.00 |
| ERCC1; PAR; FANCD2; PMK2 | 1.11E−02 | 0.69 | 0.67 | 0.36 | 0.50 | 0.80 | 0.39 | 0.78 | 2.50 |
| NQO1; RAD51; MLH1; PAR | 1.11E−02 | 0.64 | 0.55 | 0.25 | 0.53 | 0.81 | 0.37 | 0.58 | 2.84 |
| RAD51; XPF; PAR; PARP1 | 1.12E−02 | 0.64 | 0.34 | 0.31 | 0.71 | 0.67 | 0.32 | 0.47 | 2.14 |
| NQO1; RAD51; XPF; PAR | 1.12E−02 | 0.66 | 0.38 | 0.25 | 0.61 | 0.76 | 0.31 | 0.44 | 2.60 |
| ERCC1; RAD51; MLH1; PMK2 | 1.20E−02 | 0.70 | 0.58 | 0.27 | 0.50 | 0.84 | 0.38 | 0.61 | 3.17 |
| ERCC1; NQO1; RAD51; PARP1 | 1.22E−02 | 0.67 | 0.71 | 0.29 | 0.42 | 0.86 | 0.45 | 0.78 | 2.96 |
| BRCA1; XPF; PAR; PARP1 | 1.24E−02 | 0.66 | 0.33 | 0.44 | 0.63 | 0.71 | 0.32 | 0.59 | 2.15 |
| MLH1; PMK2; PARP1; ATM | 1.29E−02 | 0.70 | 0.60 | 0.29 | 0.50 | 0.83 | 0.39 | 0.66 | 3.00 |
| ERCC1; MLH1; PMK2; PARP1 | 1.29E−02 | 0.69 | 0.61 | 0.26 | 0.50 | 0.83 | 0.39 | 0.63 | 3.00 |
| RAD51; BRCA1; FANCD2; ATM | 1.33E−02 | 0.68 | 0.52 | 0.37 | 0.54 | 0.82 | 0.34 | 0.64 | 2.95 |
| BRCA1; XPF; FANCD2; ATM | 1.34E−02 | 0.69 | 0.48 | 0.45 | 0.54 | 0.81 | 0.32 | 0.68 | 2.91 |
| BRCA1; XPF; MLH1; ATM | 1.34E−02 | 0.67 | 0.42 | 0.45 | 0.59 | 0.75 | 0.31 | 0.64 | 2.36 |
| ERCC1; NQO1; BRCA1; PAR | 1.35E−02 | 0.66 | 0.69 | 0.31 | 0.45 | 0.84 | 0.43 | 0.79 | 2.88 |
| ERCC1; NQO1; BRCA1; PMK2 | 1.37E−02 | 0.69 | 0.72 | 0.34 | 0.43 | 0.83 | 0.44 | 0.86 | 2.60 |
| RAD51; MLH1; PAR; PMK2 | 1.38E−02 | 0.70 | 0.62 | 0.20 | 0.58 | 0.83 | 0.35 | 0.55 | 3.48 |
| ERCC1; XPF; PAR; ATM | 1.39E−02 | 0.65 | 0.36 | 0.33 | 0.63 | 0.75 | 0.31 | 0.49 | 2.50 |
| MLH1; PAR; PMK2; PARP1 | 1.40E−02 | 0.67 | 0.55 | 0.27 | 0.48 | 0.87 | 0.40 | 0.62 | 3.64 |
| BRCA1; PAR; PARP1; ATM | 1.50E−02 | 0.58 | 0.24 | 0.20 | 0.64 | 0.82 | 0.27 | 0.30 | 3.50 |
| ERCC1; RAD51; FANCD2; ATM | 1.52E−02 | 0.67 | 0.57 | 0.36 | 0.47 | 0.86 | 0.38 | 0.71 | 3.29 |
| ERCC1; NQO1; PARP1; ATM | 1.53E−02 | 0.64 | 0.60 | 0.37 | 0.45 | 0.80 | 0.42 | 0.77 | 2.25 |
| ERCC1; BRCA1; PAR; PMK2 | 1.58E−02 | 0.70 | 0.71 | 0.32 | 0.48 | 0.83 | 0.42 | 0.80 | 2.86 |
| ERCC1; PAR; FANCD2; PARP1 | 1.61E−02 | 0.68 | 0.67 | 0.35 | 0.51 | 0.81 | 0.38 | 0.75 | 2.70 |
| ERCC1; RAD51; MLH1; PAR | 1.62E−02 | 0.70 | 0.43 | 0.27 | 0.55 | 0.82 | 0.33 | 0.49 | 3.09 |
| ERCC1; NQO1; PAR; ATM | 1.70E−02 | 0.61 | 0.57 | 0.38 | 0.42 | 0.85 | 0.43 | 0.79 | 2.81 |
| ERCC1; NQO1; RAD51; PAR | 1.72E−02 | 0.69 | 0.75 | 0.31 | 0.44 | 0.84 | 0.45 | 0.84 | 2.77 |
| RAD51; BRCA1; MLH1; ATM | 1.73E−02 | 0.67 | 0.37 | 0.28 | 0.55 | 0.79 | 0.33 | 0.47 | 2.61 |
| ERCC1; RAD51; PAR; FANCD2 | 1.74E−02 | 0.75 | 0.63 | 0.35 | 0.52 | 0.81 | 0.37 | 0.72 | 2.70 |
| ERCC1; RAD51; MLH1; PARP1 | 1.75E−02 | 0.68 | 0.35 | 0.30 | 0.52 | 0.78 | 0.34 | 0.48 | 2.41 |
| NQO1; BRCA1; XPF; PAR | 1.77E−02 | 0.68 | 0.37 | 0.28 | 0.61 | 0.74 | 0.32 | 0.46 | 2.32 |
| ERCC1; MLH1; PAR; PMK2 | 1.80E−02 | 0.68 | 0.54 | 0.29 | 0.44 | 0.88 | 0.42 | 0.65 | 3.53 |
| NQO1; BRCA1; PMK2; PARP1 | 1.84E−02 | 0.66 | 0.78 | 0.31 | 0.42 | 0.86 | 0.47 | 0.90 | 2.92 |
| RAD51; BRCA1; PAR; PMK2 | 1.86E−02 | 0.68 | 0.52 | 0.19 | 0.58 | 0.82 | 0.35 | 0.49 | 3.17 |
| RAD51; BRCA1; XPF; FANCD2 | 1.88E−02 | 0.70 | 0.45 | 0.46 | 0.48 | 0.81 | 0.34 | 0.70 | 2.57 |
| ERCC1; BRCA1; PAR; PARP1 | 1.89E−02 | 0.66 | 0.28 | 0.25 | 0.53 | 0.87 | 0.30 | 0.38 | 4.00 |
| ERCC1; BRCA1; PMK2; PARP1 | 1.89E−02 | 0.69 | 0.66 | 0.28 | 0.50 | 0.80 | 0.40 | 0.70 | 2.50 |
| RAD51; PAR; FANCD2; ATM | 1.92E−02 | 0.71 | 0.56 | 0.33 | 0.56 | 0.82 | 0.34 | 0.64 | 3.15 |
| BRCA1; PAR; FANCD2; ATM | 1.93E−02 | 0.69 | 0.44 | 0.38 | 0.55 | 0.83 | 0.32 | 0.60 | 3.27 |
| ERCC1; NQO1; PAR; PMK2 | 1.98E−02 | 0.63 | 0.79 | 0.31 | 0.45 | 0.83 | 0.45 | 0.88 | 2.69 |
| NQO1; MLH1; PARP1; ATM | 2.07E−02 | 0.65 | 0.58 | 0.37 | 0.46 | 0.77 | 0.42 | 0.76 | 2.00 |
| ERCC1; NQO1; RAD51; PMK2 | 2.07E−02 | 0.64 | 0.72 | 0.32 | 0.43 | 0.83 | 0.45 | 0.85 | 2.45 |
| RAD51; BRCA1; PMK2; PARP1 | 2.09E−02 | 0.66 | 0.34 | 0.19 | 0.55 | 0.79 | 0.35 | 0.38 | 2.57 |
| ERCC1; MLH1; PAR; PARP1 | 2.13E−02 | 0.65 | 0.48 | 0.27 | 0.50 | 0.82 | 0.38 | 0.56 | 2.83 |
| NQO1; RAD51; MLH1; PMK2 | 2.20E−02 | 0.68 | 0.63 | 0.32 | 0.43 | 0.83 | 0.44 | 0.77 | 2.45 |
| BRCA1; XPF; PARP1; ATM | 2.27E−02 | 0.66 | 0.32 | 0.40 | 0.53 | 0.79 | 0.32 | 0.55 | 2.46 |
| BRCA1; XPF; FANCD2; PARP1 | 2.41E−02 | 0.72 | 0.47 | 0.42 | 0.50 | 0.79 | 0.36 | 0.68 | 2.42 |
| BRCA1; PMK2; PARP1; ATM | 2.43E−02 | 0.67 | 0.53 | 0.24 | 0.52 | 0.80 | 0.39 | 0.57 | 2.58 |
| ERCC1; MLH1; PARP1; ATM | 2.43E−02 | 0.65 | 0.50 | 0.33 | 0.48 | 0.78 | 0.39 | 0.64 | 2.23 |
| RAD51; BRCA1; XPF; ATM | 2.44E−02 | 0.65 | 0.33 | 0.35 | 0.56 | 0.79 | 0.31 | 0.49 | 2.67 |
| MLH1; PAR; PMK2; ATM | 2.45E−02 | 0.67 | 0.57 | 0.30 | 0.50 | 0.81 | 0.40 | 0.68 | 2.67 |
| ERCC1; FANCD2; PMK2; ATM | 2.45E−02 | 0.69 | 0.64 | 0.37 | 0.50 | 0.77 | 0.40 | 0.78 | 2.20 |
| BRCA1; MLH1; PAR; ATM | 2.61E−02 | 0.65 | 0.38 | 0.27 | 0.58 | 0.75 | 0.34 | 0.48 | 2.32 |
| ERCC1; RAD51; XPF; ATM | 2.66E−02 | 0.66 | 0.34 | 0.30 | 0.59 | 0.74 | 0.32 | 0.47 | 2.25 |
| XPF; FANCD2; PARP1; ATM | 2.86E−02 | 0.69 | 0.48 | 0.37 | 0.50 | 0.83 | 0.35 | 0.64 | 2.88 |
| NQO1; BRCA1; PARP1; ATM | 2.94E−02 | 0.64 | 0.32 | 0.31 | 0.50 | 0.77 | 0.36 | 0.49 | 2.20 |
| NQO1; PAR; PMK2; ATM | 3.00E−02 | 0.60 | 0.68 | 0.33 | 0.48 | 0.82 | 0.42 | 0.81 | 2.69 |
| NQO1; MLH1; PAR; PARP1 | 3.04E−02 | 0.61 | 0.53 | 0.24 | 0.53 | 0.75 | 0.39 | 0.57 | 2.13 |
| NQO1; PMK2; PARP1; ATM | 3.14E−02 | 0.65 | 0.73 | 0.35 | 0.45 | 0.78 | 0.44 | 0.88 | 2.07 |
| ERCC1; BRCA1; XPF; ATM | 3.32E−02 | 0.67 | 0.37 | 0.30 | 0.58 | 0.73 | 0.34 | 0.49 | 2.12 |
| RAD51; BRCA1; PMK2; ATM | 3.35E−02 | 0.65 | 0.47 | 0.22 | 0.52 | 0.79 | 0.39 | 0.51 | 2.42 |
| RAD51; XPF; PAR; ATM | 3.42E−02 | 0.62 | 0.36 | 0.33 | 0.71 | 0.65 | 0.32 | 0.50 | 2.05 |
| ERCC1; MLH1; PMK2; ATM | 3.47E−02 | 0.68 | 0.55 | 0.31 | 0.47 | 0.80 | 0.41 | 0.68 | 2.35 |
| ERCC1; BRCA1; MLH1; PAR | 3.58E−02 | 0.66 | 0.41 | 0.24 | 0.52 | 0.80 | 0.37 | 0.48 | 2.61 |
| RAD51; XPF; FANCD2; PARP1 | 3.62E−02 | 0.70 | 0.44 | 0.43 | 0.45 | 0.81 | 0.37 | 0.69 | 2.33 |
| RAD51; BRCA1; MLH1; PAR | 3.70E−02 | 0.68 | 0.28 | 0.16 | 0.50 | 0.80 | 0.38 | 0.33 | 2.50 |
| ERCC1; RAD51; PMK2; PARP1 | 3.74E−02 | 0.66 | 0.65 | 0.22 | 0.48 | 0.81 | 0.43 | 0.64 | 2.54 |
| ERCC1; PAR; PMK2; PARP1 | 3.87E−02 | 0.66 | 0.71 | 0.29 | 0.45 | 0.82 | 0.44 | 0.79 | 2.58 |
| NQO1; XPF; PAR; PARP1 | 4.10E−02 | 0.65 | 0.40 | 0.25 | 0.60 | 0.68 | 0.36 | 0.48 | 1.90 |
| ERCC1; NQO1; PAR; PARP1 | 4.17E−02 | 0.64 | 0.69 | 0.29 | 0.43 | 0.79 | 0.46 | 0.80 | 2.07 |
| ERCC1; RAD51; PARP1; ATM | 4.25E−02 | 0.62 | 0.07 | 0.25 | 1.00 | 0.74 | 0.24 | 0.25 | 3.80 |
| ERCC1; FANCD2; PARP1; ATM | 4.44E−02 | 0.65 | 0.54 | 0.36 | 0.44 | 0.82 | 0.41 | 0.72 | 2.43 |
| NQO1; RAD51; PMK2; PARP1 | 4.46E−02 | 0.62 | 0.75 | 0.31 | 0.39 | 0.82 | 0.49 | 0.91 | 2.16 |
| RAD51; PAR; PMK2; PARP1 | 4.74E−02 | 0.64 | 0.59 | 0.20 | 0.50 | 0.77 | 0.43 | 0.60 | 2.17 |
| ERCC1; PMK2; PARP1; ATM | 4.77E−02 | 0.65 | 0.59 | 0.25 | 0.46 | 0.81 | 0.43 | 0.65 | 2.45 |
| RAD51; PMK2; PARP1; ATM | 4.90E−02 | 0.66 | 0.60 | 0.23 | 0.47 | 0.80 | 0.43 | 0.64 | 2.37 |
| NQO1; RAD51; BRCA1; PMK2 | 5.09E−02 | 0.68 | 0.76 | 0.27 | 0.43 | 0.80 | 0.47 | 0.85 | 2.16 |
| BRCA1; MLH1; PAR; PARP1 | 5.21E−02 | 0.66 | 0.30 | 0.22 | 0.53 | 0.73 | 0.38 | 0.40 | 1.99 |
| NQO1; RAD51; PARP1; ATM | 5.33E−02 | 0.62 | 0.53 | 0.27 | 0.42 | 0.79 | 0.46 | 0.67 | 2.00 |
| XPF; MLH1; PAR; ATM | 5.41E−02 | 0.65 | 0.38 | 0.43 | 0.58 | 0.69 | 0.35 | 0.64 | 1.87 |
| ERCC1; BRCA1; MLH1; ATM | 5.51E−02 | 0.67 | 0.43 | 0.27 | 0.50 | 0.74 | 0.40 | 0.55 | 1.90 |
| ERCC1; RAD51; PAR; PMK2 | 5.65E−02 | 0.69 | 0.68 | 0.24 | 0.48 | 0.80 | 0.44 | 0.71 | 2.37 |
| ERCC1; PAR; FANCD2; ATM | 5.67E−02 | 0.64 | 0.54 | 0.36 | 0.44 | 0.83 | 0.42 | 0.74 | 2.63 |
| NQO1; RAD51; PAR; PARP1 | 5.73E−02 | 0.59 | 0.38 | 0.23 | 0.44 | 0.80 | 0.43 | 0.49 | 2.20 |
| NQO1; BRCA1; PAR; ATM | 5.79E−02 | 0.60 | 0.34 | 0.30 | 0.53 | 0.72 | 0.38 | 0.51 | 1.89 |
| NQO1; RAD51; PMK2; ATM | 5.95E−02 | 0.63 | 0.70 | 0.31 | 0.45 | 0.76 | 0.46 | 0.83 | 1.88 |
| ERCC1; PAR; PMK2; ATM | 6.00E−02 | 0.62 | 0.59 | 0.28 | 0.42 | 0.86 | 0.46 | 0.74 | 2.95 |
| BRCA1; XPF; PAR; ATM | 6.06E−02 | 0.63 | 0.31 | 0.39 | 0.64 | 0.65 | 0.35 | 0.55 | 1.86 |
| XPF; PAR; PARP1; ATM | 6.33E−02 | 0.62 | 0.31 | 0.33 | 0.64 | 0.65 | 0.35 | 0.49 | 1.85 |
| ERCC1; BRCA1; PMK2; ATM | 6.44E−02 | 0.66 | 0.66 | 0.28 | 0.46 | 0.78 | 0.44 | 0.75 | 2.09 |
| NQO1; MLH1; PAR; PMK2 | 6.59E−02 | 0.63 | 0.62 | 0.27 | 0.42 | 0.81 | 0.47 | 0.77 | 2.23 |
| ERCC1; BRCA1; PAR; ATM | 6.68E−02 | 0.62 | 0.29 | 0.25 | 0.57 | 0.73 | 0.34 | 0.40 | 2.14 |
| ERCC1; RAD51; PAR; ATM | 7.17E−02 | 0.63 | 0.15 | 0.24 | 0.67 | 0.73 | 0.29 | 0.29 | 2.50 |
| NQO1; MLH1; PAR; ATM | 7.20E−02 | 0.62 | 0.48 | 0.36 | 0.45 | 0.73 | 0.43 | 0.72 | 1.66 |
| RAD51; XPF; FANCD2; ATM | 7.40E−02 | 0.69 | 0.45 | 0.38 | 0.46 | 0.80 | 0.38 | 0.65 | 2.32 |
| ERCC1; MLH1; PAR; ATM | 8.23E−02 | 0.61 | 0.50 | 0.28 | 0.48 | 0.72 | 0.43 | 0.64 | 1.74 |
| ERCC1; RAD51; MLH1; ATM | 8.26E−02 | 0.66 | 0.48 | 0.28 | 0.50 | 0.71 | 0.41 | 0.59 | 1.75 |
| BRCA1; PAR; PMK2; ATM | 8.59E−02 | 0.64 | 0.54 | 0.22 | 0.45 | 0.82 | 0.45 | 0.64 | 2.50 |
| NQO1; BRCA1; PAR; PMK2 | 8.73E−02 | 0.63 | 0.66 | 0.28 | 0.40 | 0.81 | 0.49 | 0.83 | 2.16 |
| ERCC1; RAD51; PMK2; ATM | 9.29E−02 | 0.63 | 0.62 | 0.25 | 0.41 | 0.81 | 0.48 | 0.74 | 2.18 |
| RAD51; MLH1; PAR; ATM | 9.60E−02 | 0.65 | 0.32 | 0.24 | 0.53 | 0.73 | 0.38 | 0.43 | 1.99 |
| MLH1; PAR; PARP1; ATM | 1.01E−01 | 0.62 | 0.38 | 0.28 | 0.48 | 0.76 | 0.40 | 0.53 | 2.03 |
| ERCC1; RAD51; BRCA1; ATM | 1.17E−01 | 0.62 | 0.21 | 0.26 | 0.46 | 0.78 | 0.35 | 0.37 | 2.08 |
| RAD51; BRCA1; PARP1; ATM | 1.26E−01 | 0.62 | 0.17 | 0.15 | 0.50 | 0.80 | 0.35 | 0.24 | 2.50 |
| ERCC1; PAR; PARP1; ATM | 1.37E−01 | 0.58 | 0.39 | 0.26 | 0.46 | 0.75 | 0.43 | 0.54 | 1.83 |
| NQO1; RAD51; PAR; PMK2 | 1.53E−01 | 0.62 | 0.69 | 0.25 | 0.41 | 0.75 | 0.51 | 0.84 | 1.63 |
| NQO1; PAR; PARP1; ATM | 1.66E−01 | 0.57 | 0.45 | 0.29 | 0.45 | 0.68 | 0.46 | 0.65 | 1.42 |
| NQO1; PAR; PMK2; PARP1 | 1.80E−01 | 0.58 | 0.66 | 0.25 | 0.40 | 0.75 | 0.52 | 0.83 | 1.58 |
| RAD51; PAR; PMK2; ATM | 1.85E−01 | 0.62 | 0.61 | 0.16 | 0.47 | 0.78 | 0.47 | 0.63 | 2.13 |
| ERCC1; BRCA1; PARP1; ATM | 1.92E−01 | 0.64 | 0.23 | 0.26 | 0.47 | 0.74 | 0.38 | 0.40 | 1.77 |
| PAR; PMK2; PARP1; ATM | 2.03E−01 | 0.63 | 0.57 | 0.19 | 0.46 | 0.73 | 0.48 | 0.65 | 1.68 |
| ERCC1; RAD51; BRCA1; PARP1 | 2.28E−01 | 0.67 | 0.16 | 0.21 | 0.42 | 0.81 | 0.36 | 0.30 | 2.22 |
| NQO1; RAD51; PAR; ATM | 2.48E−01 | 0.59 | 0.46 | 0.24 | 0.42 | 0.69 | 0.49 | 0.64 | 1.34 |

We claim:

1. A method with a predetermined level of predictability for assessing a risk of development of a triple negative breast cancer or a recurrence of triple negative breast cancer in a subject comprising:
a. measuring the level of an effective amount of two or more TNBCMARKERS selected from the group consisting of FANCD2, XPF, pMK2, PAR, PARP1, MLH1, ATM, RAD51, BRCA1, ERCC1, NQO1, p53, Ki67, in a sample from the subject, and
b. measuring a clinically significant alteration in the level of the two or more
TNBCMARKERS in the sample, wherein the alteration indicates an increased risk of developing a triple negative breast cancer in the subject.

2. The method of claim 1, wherein the TNBCMARKERS is a DNA repair protein are selected from the group consisting of FANCD2, XPF, pMK2, PAR, PARP1, MLH, ATM, RAD51, BRCA1, and ERCC1.

3. The method of claim 2, further comprising detecting one or more TNBCMARKERS selected from the group consisting of NQO1, p53, and Ki67.

4. The method claim one wherein at least one TNBCMARKER is FANDC2, BRCA1, or RAD51 and at least one TNBCMARKER is
a. XPF or ERCC1;
b. pMK2 or ATM; or
c. PAR or PARP1.

5. The method of claim 4, further comprising detecting of or more TNBCMARKERS selected from the group consisting of NQO1, p53, and Ki67.

6. The method of claim 2, wherein the two TNBCMARKERS are DNA repair proteins belonging to different DNA repair pathways.

7. The method of claim 2, comprising detecting three or more TNBCMARKERS wherein said TNBCMARKERS belonging to two or more different DNA repair pathways

8. The method of claim 2, comprising detecting four or more TNBCMARKERS wherein said TNBCMARKERS belonging to two or more different DNA repair pathways.

9. The method of claim 2, comprising detecting four or more TNBCMARKERS wherein said TNBCMARKERS belonging to three or more different DNA repair pathways.

10. The method of claim 1, comprising detecting
a. FAND2 and at least one TNBCMARKER selected from the group consisting of XPF, pMK2, PAR, PARP1, MLH1, ATM, RAD51, BRCA1, and ERCC1;
b. XPF and at least one TNBCMARKER selected from the group consisting of FANCD2, pMK2, PAR, PARP1, MLH1, ATM, RAD51, BRCA1, and ERCC1;
c. pMK2 and at least one TNBCMARKER selected from the group consisting of FANCD2, XPF, PAR, PARP1, MLH1, ATM, RAD51, BRCA1, and ERCC1;
d. PAR and at least one TNBCMARKER selected from the group consisting of FANCD2, XPF, pMK2, PARP1, MLH1, ATM, RAD51, BRCA1, and ERCC1;
e. PARP1 and at least one TNBCMARKER selected from the group consisting of FANCD2, XPF, pMK2, PAR, MLH1, ATM, RAD51, BRCA1, and ERCC1;
f. MLH1 and at least one TNBCMARKER selected from the group consisting of FANCD2, XPF, pMK2, PAR, PARP1, ATM, RAD51, BRCA1, and ERCC1;
g. ATM and at least one TNBCMARKER selected from the group consisting of FANCD2, XPF, pMK2, PAR, PARP1, MLH1, RAD51, BRCA1, and ERCC1;
h. RAD51 and at least one TNBCMARKER selected from the group consisting of FANCD2, XPF, pMK2, PAR, PARP1, MLH1, ATM, BRCA1, and ERCC1;
i. BRCA1 and at least one FANCD2, XPF, pMK2, PAR, PARP1, MLH1, ATM, RAD51, and ERCC1; or
j. ERCC1 and at least one FANCD2, XPF, pMK2, PAR, PARP1, MLH1, ATM, RAD51, and BRCA1.

11. The method of claim 10, further comprising detecting of or more TNBCMARKERS selected from the group consisting of NQO1, p53, and Ki67.

12. The method of claim 1, further comprising measuring at least one standard parameters associated with said triple negative breast cancer.

13. The method of claim 1, wherein the level of expression of XPF, FANCD2, PAR and pMK2 is measured.

14. The method of claim 1, wherein the level of a TNBCMARKER is measured immunochemically.

15. The method of claim 14, wherein the immunochemical detection is by radioimmunoassay, immunofluorescence, quantum dot, electrochemical, oligonucleotide-conjugated PCR amplification and detection assay, or by an enzyme-linked immunosorbent assay.

16. The method of claim 1, wherein the sample is a tumor biopsy.

17. The method of claim 1, wherein said biopsy is a fine needle aspirate, a core biopsy, an excisional tissue biopsy or an incisional tissue biopsy.

18. The method of claim 1, wherein said sample is a tumor cell from blood, lymph nodes, or bodily fluid

19. A method with a predetermined level of predictability for assessing for assessing a risk of development of a triple negative breast cancer in a subject comprising:
a. measuring the level of an effective amount of two or more TNBCMARKERS selected from the group consisting of XPF, pMK2, PAR, PARP1, MLH, FANCD2, ATM, RAD51, BRCA1, ERCC1, NQO1, p53, Ki67 in a sample from the subject, and
b. comparing the level of the effective amount of the two or more TNBCMARKERS to a reference value.

20. The method of claim 19, wherein the reference value is an index value.

21. A method with a predetermined level of predictability for assessing the progression of a triple negative breast cancer in a subject comprising:
a. detecting the level of an effective amount of two or more TNBCMARKERS selected from the group consisting of XPF, pMK2, PAR, PARP1, MLH, FANCD2, ATM, RAD51, BRCA1, ERCC1, NQO1, p53, Ki67 in a first sample from the subject at a first period of time;
b. detecting the level of an effective amount of two or more TNBCMARKERS in a second sample from the subject at a second period of time;
c. comparing the level of the effective amount of the two or more TNBCMARKERS detected in step (a) to the amount detected in step (b), or to a reference value.

22. The method of claim 19, wherein the first sample is taken from the subject prior to being treated for the triple negative breast cancer.

23. The method of claim 19, wherein the second sample is taken from the subject after being treated for the triple negative breast cancer.

24. A method with a predetermined level of predictability for monitoring the effectiveness of treatment for a triple negative breast cancer:
a. detecting the level of an effective amount of two or more TNBCMARKERS selected from the group consisting of XPF, pMK2, PAR, PARP1, MLH, FANCD2, ATM, RAD51, BRCA1, ERCC1, NQO1, p53, Ki67 in a first sample from the subject at a first period of time;
b. detecting the level of an effective amount of two or more TNBCMARKERS in a second sample from the subject at a second period of time;
c. comparing the level of the effective amount of the two or more TNBCMARKERS detected in step (a) to the amount detected in step (b), or to a reference value, wherein the effectiveness of treatment is monitored by a change in the level of the effective amount of two or more TNBCMARKERS from the subject.

25. The method of claim 24, wherein the subject has previously been treated for the triple negative breast cancer.

26. The method of claim 24, wherein the first sample is taken from the subject prior to being treated for the triple negative breast cancer.

27. The method of claim 24, wherein the second sample is taken from the subject after being treated for the triple negative breast cancer.

28. A method with a predetermined level of predictability for selecting a treatment regimen for a subject diagnosed with a triple negative breast cancer comprising:
a. detecting the level of an effective amount of two or more TNBCMARKERS selected from the group consisting of XPF, pMK2, PAR, PARP1, MLH, FANCD2, ATM, RAD51, BRCA1, ERCC1, NQO1, p53, Ki67 in a first sample from the subject at a first period of time;
b. optionally detecting the level of an effective amount of two or more TNBCMARKERS in a second sample from the subject at a second period of time;
c. comparing the level of the effective amount of the two or more TNBCMARKERS detected in step (a) to a reference value, or optionally, to the amount detected in step (b).

29. The method of claim 28, wherein the subject has previously been treated for the triple negative breast cancer.

30. The method of claim 28, wherein the first sample is taken from the subject prior to being treated for the tumor.

31. The method of claim 28, wherein the second sample is taken from the subject after being treated for the triple negative breast cancer.
